(19) United States
(12) Patent Application Publication
(10) Pub. No.: US 2025/0011880 A1
(43) Pub. Date: Jan. 9, 2025

(54) USE OF A TRANSCRIPTOMIC SIGNATURE BASED ON HERVs EXPRESSION TO CHARACTERIZE NEW ACUTE MYELOID LEUKEMIA SUBTYPES

(71) Applicants: ERVIMMUNE, LYON CEDEX 08 (FR); CENTRE LEON BERARD, LYON (FR); UNIVERSITE CLAUDE BERNARD LYON 1, Villeurbanne (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(72) Inventors: Stéphane DEPIL, LYON CEDEX 08 (FR); Vincent ALCAZER, PIERRE-BENITE (FR)

(73) Assignees: ERVIMMUNE, LYON CEDEX 08 (FR); CENTRE LEON BERARD, LYON (FR); UNIVERSITE CLAUDE BERNARD LYON 1, Villeurbanne (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(21) Appl. No.: 18/713,351

(22) PCT Filed: Nov. 25, 2022
(86) PCT No.: PCT/EP2022/083376

§ 371 Date: May 24, 2024

(30) Foreign Application Priority Data
Nov. 26, 2021 (EP) 21306649.1

(51) Int. Cl. C12Q 1/6886; C12Q 1/6869; C12Q 1/70; G16B 20/00

(52) U.S. Cl. CPC C12Q 1/6886; C12Q 1/6869; C12Q 1/701; G16B 20/00

(57) ABSTRACT

The use of a transcriptomic signature based on HERVs expression to characterize new AML subtypes, and a method to determine to which AML subtype a patient pertains. The method includes providing relationship between the 9 AML sub-types and HERVs characterized by their specific herv_id and their relationship with one of these AML subtypes, determining from a patient cell sample HERVs expression profile, determining which of the 9 AML subtypes is the most represented based on HERV expression in the cell sample, and attributing to the patient the most represented AML subtype among the 9 AML subtypes. The method allows identifying patients with medium good or bad prognosis and treating the same with a cancer therapy against AML.

### FIELD OF INVENTION

The present invention concerns the use of a transcriptomic signature based on Human endogenous retroviruses (HERVs) expression to characterize new acute myeloid leukemia (AML) subtypes, and a method to determine to which AML subtype a patient pertains.

### BACKGROUND OF INVENTION

Human endogenous retroviruses (HERVs) represent 8% of the human genome (1). These sequences are remnants of ancestral germline infections by exogenous retroviruses (2). The original sequence of a HERV is that of an exogenous retrovirus, with two promoter long-terminal repeat (LTR) sequences surrounding the virus open-reading frames (ORFs): gag, pro, pol and env (3). However, after millions of years of evolution, these ORFs have been deeply altered, and there is currently no description of any autonomous fully infectious HERV (4).

The long-standing belief is that HERVs are repressed by epigenetic mechanisms and are thus not expressed, or only poorly, in normal tissues (5). However, recent studies have shown that HERV expression can be detected in a vast range of normal tissues (6). Different pathological conditions can lead to aberrant HERV expression, as it has now been largely described in auto-immune diseases (4) and in cancers (7), where HERVs have been the subject of many studies over the last years. Indeed, it was reported that HERVs could participate in oncogenesis by inducing chromosomal instability, promoting aberrant gene expression with their LTR or by impacting the immune system with their RNA and protein products (7). HERVs could thus play a prominent role in cancer immunity, increasing tumor immunogenicity by promoting (i) an innate immune response triggered by the viral defense pathway induced by their nucleic acid intermediates, and (ii) an adaptive immune response by forming a pool of tumor-associated antigens (8).

Acute Myeloid Leukemia (AML) is a heterogeneous disease characterized by the clonal expansion of myeloid progenitor and stem cells (9). While some AML subtypes are characterized by recurrent genetic translocations or mutations associated with particular prognoses, most AMLs present a normal or complex karyotype, and identifying key factors that predict treatment resistance in these patients represents a major challenge (9, 10). Aside from disease stratification, AML also belongs to malignancies with the lowest mutational burdens (11), and finding tumor-specific antigens for immunotherapeutic approaches remains very difficult as the frequency of mutations creating neoantigens is expected to be low. In this context, HERV-derived antigens could represent a unique source of non-conventional epitopes that could be exploited for the development of new immunotherapies (12).

To date, little is known about the expression of HERVs in AML and its relevance as either a biomarker or a therapeutic target. Evidence of HERV-K/HML-2 expression in AML cells was shown as early as 1993 and confirmed in the early 2000s (13, 14). Few studies then focused on HERVs in AML until the late 2010s, with the demonstration that azacytidine (Aza) activates the transcription of different HERVs, potentially contributing to its clinical effects (15). The exact role of HERVs in Aza therapy is however a matter of debate, with recent evidence arguing in favor of a HERV-independent therapeutic effect (16). More recently, a link was established between HERVs and the expression of surrounding genes in AML, suggesting a regulatory role of these retroelements (17). Albeit, few data exist on HERV expression and their immune impact in AML, with studies relying on non-exhaustive quantification methods, such as polymerase chain reaction (PCR), or focusing only on a few HERV loci.

### SUMMARY OF THE INVENTION

In this invention, we established different signatures based on HERV expression from several RNA sequencing data to characterize different AML subtypes. 9 clusters or AML subtypes have been characterized. Relying only on HERV expression, it was possible to define these new AML subtypes independently of karyotype of molecular alterations. These results suggest that HERVs can be used to improve risk stratification and treatment resistance prediction, in particular in patients with no genetic or molecular abnormalities associated with well-defined prognosis and resistance profiles. These 9 subtypes were associated with significant differences in overall survival (OS) and Hazard ratio (HR) among intensively treated patient, independently of established prognosis factors such as age, ELN2017 and white blood count, integrated in a multivariate Cox model. These clusters also presented distinct cancer hallmark profiles, as assessed by single sample gene-set variation analysis (GSVA) based on cancer hallmark signatures.

It is first provided 9 AML subtypes defined by representative HERVs as disclosed in Table 1. The first column of Table 1 indicates the cluster number (1, 2, 3, 4, 5, 6, 7, 8 and 9) to which the HERV of column 2 pertains. It is then provided a method for attributing a patient to an AML subtype among 9 AML subtypes characterized by their specific HERVs listed in Table 1 with the indication of their herv_id and of their locus in the GRCH38 version of the human genome.

The present invention thus relates to a method for attributing an AML patient to an AML subtype among 9 AML subtypes characterized by their specific HERVs listed in Table 1 with the indication of their herv_id (and of their locus in the GRCH38 version of the human genome). The method comprises considering or providing relationship between said 9 AML sub-types and HERVs characterized by their specific herv_id, their locus in the GRCH38 version of the human genome and their relationship with one of these AML subtypes, as set forth in Table 1. The method comprises determining HERVs expression profile in a patient cell sample. The expression profile is the expression level for HERVs as set forth in Table 1. This step allows to determine which of the 9 AML subtypes (or the HERVs corresponding to this subtype) is the most represented based on HERV expression in the sample cells. In particular, HERV expression is related to, and determinable by, HERV RNA in the cell sample, such as bone marrow sample. Based on Table 1 and HERV expression, the method comprises attributing to the patient an AML subtype among the 9 AML subtypes. As explained later, each one subtype may correspond to a given prognosis, so that the method may further comprise attributing a particular prognosis, which is the one corresponding to the attributed sub-type. Also, as explained later, the method may comprise treating the patient with a cancer therapy suited to its prognosis resulting from the preceding steps.

The method comprises attributing to the patient an AML subtype among the 9 AML subtypes. This subtype is the most represented in the cell sample based on HERV expression profile. It is the most represented in the cell sample as determined by identifying and quantifying the HERVs pertaining to the sub-type. In an aspect, the method may comprise
a. determining from a patient's sample the expression value of the 703 HERVs listed in Table 1, or of a sub-part of these 703 HERVs,
b. multiplying each HERV expression value by the coefficient attributed to the corresponding HERV in Table 1,
c. for each of the 9 AML subtypes, calculate their score as the mean of each HERV expression specific to the subtype, and
d. attributing to the patient the AML subtype with the highest score among the 9 AML subtypes.

### DETAILED DESCRIPTION

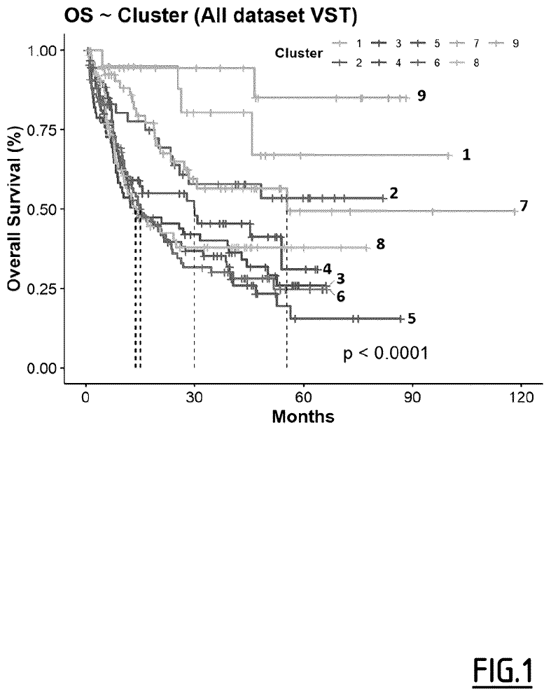

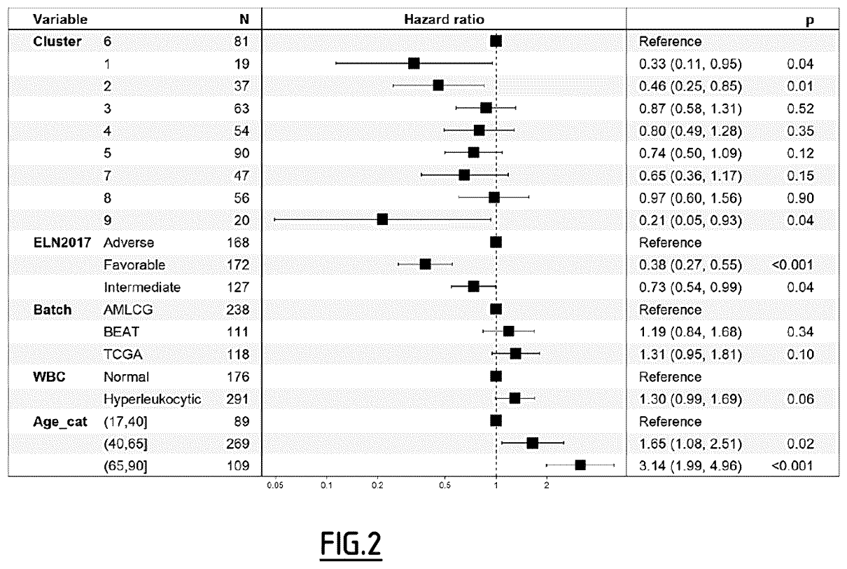

For each of the 4 public datasets (AMLCG, TCGA, BEAT and LEUCEGENE), DESEQ2 VST normalized expression data were independently calculated and further center-scaled for each dataset to correct the potential batch effect. The top 2,000 most variable HERVs was then selected independently for each dataset based on the scaled DESEQ2 VST normalized count. The 4 datasets were then merged, keeping only the intersect between each top 2,000 candidate HERVs, resulting in 961 variable HERVs conserved across the 4 datasets. Unsupervised hierarchical clustering guided by the average silhouette and Bayesian Information Criterion (BIC) evolution defined 9 clusters that were not dependent on the study and presented significant differences in term of overall survival, hazard ratio and hallmarks of cancer (FIGS. 1 and 2). HERVs signatures were calculated by selecting the top expressed HERVs in each cluster compared to all the others. Only features with a fold change >1.2 or <0.8 and an adjusted p-value <0.05 were retained, leading to a final list of 703 HERVs (Table 1). Final signatures were calculated by ponderating each HERVs by its fold change for each cluster.

Table 1 presents the 703 HERVs with their identification herv_id (or gene_id) and their respective locus in the GRCH38 version of the human genome. Using the herv_id, the open-source tool Telescope made available online (Bendall Matthew L et al., (Sep. 30, 2019), PLOS Computational Biology. 2019; 15 (9): e1006453, Telescope: Characterization of the retrotranscriptome by accurate estimation of transposable element expression. (https://journals.plos.org/ploscompbiol/article/comments?id=10.1371/journal.pcbi. 1006453, https://github.com/mlbendall/telescope) and the genomic reference file in the international General Feature Format 2613_703HERVs_GRCH38_genomic_ref.gtf (available online at: github.com/VincentAlcazer/hervs_ref/blob/main/2613_703HERVs_GRCH38_genomic_ref.gtf), the skilled person has access to the exact genomic coordinates of each of the 703 HERV sequences in the GRCH38 version of the human genome, and consequently to their corresponding nucleotide sequences.

Thus, from any cell sample from an AML patient (bone marrow sample, blood sample containing white blood cells, for example) it is possible to determine the presence of specific HERVs RNAs that pertain to said 9 AML clusters or sub-types, to determine the dominant AML cluster or sub-type for the cell sample and the patient from which the cell sample originates, and then to attribute to the patient said AML cluster or sub-type. The dominant sub-type may be determined as the one for which the HERVs RNAs profile or number is the highest or the most significant among the 9 sub-types, as disclosed herein.

In an embodiment, HERVs RNAs are recovered, cDNAs are produced from RNAs, cDNA fragments are aligned to a reference human genome, followed by the determination of the number of cDNAs aligned to a sufficient number of the 703 HERVs listed in Table 1, or to a subset of the 703 HERVs, or to all the 703 HERVs. As a sub-set, one may use the HERVs with a coefficient >1.2 and the HERVs with a coefficient <0.8.

In an aspect, High-throughput sequencing with RNA, commonly referred to as RNA-Seq, involves mapping sequenced fragments of cDNA. In RNA-Seq, the RNA is fragmented and then reverse transcribed to cDNA, or is reverse transcribed and then fragmented. These fragments are then sequenced, producing reads that are aligned back to a pre-sequenced reference genome or human genome reference. The number of reads mapped to a gene is used to quantify its expression.

Thus, in an aspect, the method comprises performing RNA-Seq, preferably next generation sequencing (NGS), in a sample of a patient, preferably a bulk bone marrow sample, method in which RNA from the sample is fragmented and the fragments are reverse transcribed into cDNA fragments, or the RNA is reverse transcribed to cDNA and then fragmented to get cDNA fragments.

The size of the RNA fragments may vary in large proportion as known by the skilled person. Typically, RNA fragments may have a size of from 50 to 100 base pairs, e.g. about 75 base pairs.

Thus, the method may comprise first performing RNA-Seq, preferably next generation sequencing (NGS) in an AML patient. RNA-Seq, preferably NGS may be performed from bulk bone marrow sample. NGS can be either non-targeted (total or polyA RNA-seq) or targeted.

In an aspect, said cDNA fragments are sequenced and aligned back to a pre-sequenced reference human genome or human genome reference, using a sequence aligner, these alignments are tested for overlap with said HERVs' sequences, and the number of overlap reads mapped to a gene is registered for each HERVs' sequence giving its expression value.

Quantifying HERVs is performed from RNA-Seq or NGS data. This comprises aligning raw-reads to human genome reference using any sequence aligner, preferably a fast or ultrafast sequence aligner; such as Bowtie2, with conservative parameters: —no-unal—score-min L,0,1.6-k 100—very-sensitive-local.

A relevant description of the method is described in reference (18), the whole content of which is incorporated herein by reference.

Quantifying HERVs expression is then made using a computer implemented method or

an adequate software. The open-source tool Telescope (18) is a suitable one. A relevant description of the method is described in the previously mentioned reference (18).

Quantifying genes is also made with any suitable tool such as HTseq or featurecount.

Normalizing expression data taking genes raw count into account may then be realized.

9 clusters or subtypes have been identified and the HERVs pertaining to each cluster are presented in Table 1, and the coefficient of each HERVs is also indicated in this Table.

The HERV-LSC score is calculated for each cluster (i.e. AML subtype):
a. multiply each HERVs' normalized expression value by its coefficient provided in Table 1,
b. for each cluster, calculate its score as the mean of each pondered HERVs' expression,
c. the cluster with the highest score is attributed to the patient.

The subtype is easy to calculate or determine and can be applied to any patient using next generation sequencing (NGS) data:
1. Perform NGS in any AML patient at diagnosis.
   a. NGS is performed from bulk bone marrow sample at diagnosis.
      i. NGS can be either non-targeted (total or polyA RNA-seq) or targeted.
2. Quantify HERVs from NGS data
   a. Align raw-reads to human genome reference using any aligner such as Bowtie2 with conservative parameters:—no-unal—score-min L,0,1.6-k 100—very-sensitive-local
   b. Quantify HERVs using the open-source tool Telescope (18).
   c. Quantify genes (the 703 HERVs of Table 1) with any tool such as HTseq or featurecount.
   d. Normalize expression data taking genes raw count into account.
3. For each cluster (i.e. AML subtype), calculate its score according to the Table 1:
   a. Multiply each HERVs' normalized expression value by the coefficient attributed to the corresponding HERV in Table 1.
   b. For each of the 9 AML subtypes or clusters, calculate their score as the mean of each pondered HERVs' expression specific to the subtype.
   c. The cluster or AML subtype with the highest score among the 9 is attributed to the patient.

In case not all HERVs are available, core-HERVs with a coefficient >1.2 or <0.8 should at least be present.

The AML subtypes correspond to specific Overall survival (OS) and/or Hazard ratio (HR). Thus, attributing an AML subtype to a patient does attribute a prognosis, in particular based on OS and/or HR.

In some cases, AML subtype 1 or 9 is attributed to the patient, with a good prognosis or the best prognosis among the 9 subtypes.

In some cases, AML subtype 2 or 7 is attributed to the patient, with a medium good prognosis among the 9 subtypes.

In some cases, AML subtype 8, 4, 3, 6, 5 is attributed to the patient, with a bad or worse prognosis among the 9 subtypes.

Thus in an aspect, the method for attributing a patient to an AML subtype among the 9 AML subtypes disclosed herein is a method of attributing a prognosis to the patient relative to AML, e.g.: if the patient is attributed AML subtype 1 or 9, prognosis is good, if he is attributed AML subtype 2 or 7, prognosis is medium good, and if he is attributed AML subtype 8, 4, 3, 6 or 5, prognosis is bad.

In an aspect, the method further comprises the recommendation of treating said patient with a cancer therapy against AML, preferably an aggressive therapy, when the patient preferably intensified chemotherapy or an alternative therapy through enrollment into a clinical trial for a novel therapy, when the patient is attributed AML subtype 2 or 7, or AML subtype 8, 4, 3, 6 or 5.

In an aspect, the method further comprises the recommendation of treating said patient with an aggressive therapy, preferably intensified chemotherapy or an alternative therapy through enrollment into a clinical trial for a novel therapy, if the patient is attributed AML subtype 8, 4, 3, 6 or 5, or with a less aggressive therapy, preferably standard chemotherapy, if the patient is attributed AML subtype 9, 1, 2 or 7.

In an aspect, the invention concerns the use of a anticancer drug for treating a subject against AML, wherein the subject had been previously identified as having an AML with a medium good or a bad prognosis by use of this method.

In another aspect the invention relates to a method of treating a subject against AML, comprising treating the patient with a cancer therapy against AML, in particular an aggressive cancer therapy, wherein the subject had been previously identified as being in a medium good or bad prognosis, by use of this method of attributing a patient to an AML subtype.

In an aspect, the invention relates to a method of treating a subject against AML, comprising treating a patient with medium good or bad prognosis with a cancer therapy against AML, in particular an aggressive cancer therapy, wherein the subject had been previously identified as having an AML with a medium good or a bad prognosis by use of the method as disclosed herein.

The aggressive therapy is preferably an identified chemotherapy or an alternative therapy through enrollment into a clinical trial for a novel therapy.

In another aspect, the invention relates to a method of treating AML in a patient, comprising the steps:
(a) attributing an AML subtype 1 to 9 to said patient by using the method as disclosed herein; and
(b) treating said patient with an aggressive therapy, preferably intensified chemotherapy or an alternative therapy through enrollment into a clinical trial for a novel therapy, if the patient is attributed AML subtype 8, 4, 3, 6 or 5, or with a less aggressive therapy, preferably standard chemotherapy, if the patient is attributed AML subtype 9, 1, 2 or 7.

Therapeutics include: cytarabine, fludarabine, idarubicin, avapritinib, dasatinib, mitoxantrone, clofarabine, cladribine, azacitidine, daunorubicin, etoposide, midostaurin, sorafenib, gilteritinib, decitabine, lomustine, quizartinib, crenolanib, enasidenib, ivosidenib, venetoclax, glasdegib, antibodies such as Gemtuzumab, magrolimab, and combinations thereof (32).

The present invention will now be described in further detail, referring to the drawings.

FIG. 1: Overall survival (OS) of intensively treated patients according to the 9 clusters in the whole cohort.

FIG. 2: Multivariate Cox analysis of overall survival of intensively treated patients. Known risk factor (ELN2017 and WBC), study (batch) and clusters are integrated in the multivariate model.

### HERV RETROTRANSCRIPTOME ACCURATELY DEFINES NORMAL HEMATOPOIETIC CELL POPULATIONS

As a first step, we examined HERVs expression in the different normal hematopoietic cell populations, assuming that distinct HERVs profiles may characterize the main cell types. Using a custom pipeline based on Telescope (18), we quantified the expression of 14,968 HERVs loci in RNA-seq data from sorted bone-marrow and peripheral blood cell populations from 9 healthy donors (n=49 samples) (19). Unsupervised hierarchical clustering based on the top 20% most variable HERVs showed a robust classification of normal hematopoietic cell types with a cluster purity of 77.6% and a corrected Rand Index of 0.61. The same approach based on genes reached a purity of 65.3% with a corrected Rand Index of 0.47.

We then sought to improve the clustering with the analysis of peaks from open chromatin regions assessed by ATAC-seq. Using the HOMER package, we applied a classic human genome annotation from gencode (v33) to annotate the set of 590,650 significant non-overlapping peaks from open chromatin regions previously defined in sorted healthy donors' bone marrow and peripheral blood cells (n=80 samples) (19). As previously described, unsupervised hierarchical clustering based on promoters elements (peaks between-100 bp and 1,000 bp away from a transcription start-site (TSS)) and intergenic elements (peaks more than 1,000 bp away from any other feature) significantly improved cluster classification, with a purity reaching 81.8%. We then re-annotated these significant peaks with a custom reference consisting of the same gencode annotation concatenated with the previously used 14,968 HERVs loci from Repeatmasker. Overall annotation showed that 16% of the total significant peaks correspond to HERVs regions. One important previously reported finding is that classification based on intergenic elements only (the so-called “distal regulatory elements”) is sufficient to classify normal hematopoietic cell populations (19). Enhanced annotation of these distal regulatory elements revealed an enrichment in HERVs, with up to 37.6% of the top 500 variable intergenic peaks corresponding to a HERV region. Plot of the total aggregated count from these regions showed a gaussian distribution surrounding HERVs' TSS, confirming the good quality of the ATAC-seq signal. Clustering of samples based on active HERVs regions (AHR, defined by peaks surrounding HERVs regions +/−1000 or 3000 bp) further improved the clustering, reaching 88.3% cluster purity.

Altogether these results show that HERV retrotranscriptome can be used to characterize normal immature and mature hematopoietic cell populations. The improved clustering obtained with AHR defined on ATAC-seq data suggests that this retrotransciptomic signature may reflect epigenetic features associated with cell differentiation.

CLP: Common Lymphoid Progenitor, CMP: Common Myeloid Progenitor, Ery: Erythrocyte, GMP: Granulocyte-Macrophage Progenitor, HSC: Hematopoietic Stem cell, LMPP: lymphoid-primed multipotent progenitor, MEP: Megakaryocyte-Erythroid Progenitor, MPP: Multipotent Progenitor.

Acute Myeloid Leukemia Cells Show Distinct HERV Profiles Close to their Normal Cell of Origin

We next evaluated how HERV retrotranscriptome may help in distinguishing AML cells. We performed the same clustering approach, adding this time the 32 RNA-seq and 45 ATAC-seq bone marrow samples from 15 AML patients at diagnosis (Corces et al., 2016). Unsupervised clustering based on the top 20% variable AHR (+/−1,000 bp from a HERV TSS) in ATAC-seq resulted again in a good classification of normal and AML cells, with a slight increase in cluster purity compared to the top 20% most variable intergenic peaks. Clustering based on HERVs expression in RNA-seq yielded comparable results. Interestingly, leukemic blast cells (blasts) clustered with either monocytes or granulocyte-monocyte progenitor (GMP) cells, LSCs with either GMP or lymphoid-primed multipotent progenitor (LMPP) cells and pre-leukemic hematopoietic stem cells (pHSCs) with either GMP or HSC/multipotent progenitor (MPP) cells, suggesting a clustering with their cell of origin as already described by Corces et al (Corces et al., 2016). Cluster purity based on the original cell categories do not consider these similarities and is thus a poor indicator of clustering performance in this case. Differential ATAC-count analysis centered on extended AHR (+/−20,000 bp from a HERV TSS) revealed distinct profiles between AML LSCs, blasts and pHSCs compared to their normal counterpart, with globally a chromatin more open in blasts and more closed in LSCs and pHSCs. To further characterize the role of HERVs in these AHR, we computed correlations between RNA expression of each HERV present in an AHR and its respective surrounding genes located at +/−50,000 bp. Strikingly, we found mostly positive correlations between HERVs expression and their surrounding genes. Annotation of the genes with a pre-established list of cancer-associated genes from the Conser Gene Census database (20) found several genes positively correlated with HERVs expressed in AHR. Of note, the highest correlation was found for GATA with ERVLB4_Xp11.23b (Pearson's R: 0.74, adjusted p-value: 8.11e-14). Using TCGA LAML RNAseq data, we then explored the association between each HERV located in an AHR and gene copy number variation (CNV) on the same cytoband. We found several HERVs correlating both positively and negatively with deletions, and mostly positively with amplifications on the same cytoband. These results show that HERVs expression profile differs according to the AML cell type and suggest that HERVs are associated with gene regulation.

AML: Acute Myeloid Leukemia, BM: Bone Marrow, CLP: Common Lymphoid Progenitor, CMP: Common Myeloid Progenitor, CNV: Copy Number Variation, Ery: Erythrocyte, FDR: False Discovery Rate, GMP: Granulocyte-Macrophage Progenitor, HSC: Hematopoietic Stem cell, LMPP: lymphoid-primed multipotent progenitor, LSC: Leukemic Stem Cell, MEP: Megakaryocyte-Erythroid Progenitor, MPP: Multipotent Progenitor, pHSC: pre-leukemic Hematopoietic Stem Cell.

HERVs Expression Defines Subtypes of AML with Distinct Cancer Hallmarks and Outcomes

After having established that HERV retrotranscriptome characterizes particular cell types, we asked whether HERVs expression could distinguish distinct AML profiles in bulk RNA-seq data. We explored HERVs expression in 4 independent RNA-seq datasets (TCGA, AMLCG, LEUCEGENE and BEAT), keeping only bone marrow samples from AML patients at diagnosis (n=788). For each dataset, we selected the top 2,000 most variables HERVs based on the scaled DESEQ2 VST normalized count. We merged the 4 datasets, keeping only the intersect between each top 2,000 candidates HERVs, resulting in 961 variable HERVs conserved across the 4 datasets. Unsupervised hierarchical clustering guided by the average silhouette evolution defined 9 clusters that were not dependent on the study. These 9 clusters were associated with significant differences in overall survival among intensively treated patients (FIG. 1), independently of established prognosis factors such as age, ELN2017 and white blood count, integrated in a multivariate Cox model (FIG. 2). These clusters also presented distinct cancer hallmark profiles, as assessed by single sample gene-set variation analysis (GSVA) based on cancer hallmark signatures. GSVA of immune signatures revealed no clear immune subtype profiles, distinguishing clusters with globally low or high immune scores.

We then assessed whether these clusters were associated with known recurrent translocations or gene mutations. We found a clear enrichment of inv(16), t(8;21) and t(15;17) in clusters 1, 7 and 9, respectively. Other karyotypes (such as complex, poor or intermediate abnormalities) showed no particular association with any cluster, underlining the heterogeneous composition of these groups. Regarding gene mutations, we found an enrichment of TP53 mutation in cluster 3.

Focusing on HERVs discriminating each cluster (i.e. HERVs overexpressed in a given cluster compared to all the others), we found that clusters 8 and 9 had the highest number of different discriminating HERVs, whereas only few HERVs discriminated clusters 3 and 5. HERVs from the HERV-H, ERV-L, MER4, HERV-L and HERV-K families were the most frequent HERVs discriminating clusters. When reported to the total number of HERVs per family, HERV-S, HERV-E, HERV-P, ERVI and HARLEQUIN families were the most frequently represented.

LSC: Leukemic Stem Cell, ROC: Receiver Operating Characteristic Curve, ssGSVA: Single Sample Genes-set Variation Analysis, WBC: White Blood Count.

### Methods

### Raw RNAseq Data

Raw RNA-seq data files were accessed from the NCBI Gene Expression Omnibus (GEO) portal, under the accession numbers GSE74246 for the sorted hematopoietic normal and AML cells from Corces et al. (19), GSE49642, GSE52656, GSE62190, GSE66917, GSE67039 and GSE106272 for the LEUCEGENE datasets, GSE127825 and GSE127826 for the six mTECs samples (6). TCGA LAML (31) and BEAT-AML (21) data were accessed from the NCI Genomic Data Commons (GDC) data portal (https://portal.gdc.cancer.gov/). Raw data for the AMLCG cohort(10) were directly provided by the AMLCG group.

### HERVs and Genes Expression Quantification

HERVs expression was quantified using a custom pipeline derived from Telescope (18). Briefly, RNAseq reads were aligned to a custom transcriptome using bowtie2 v2.2.1 (22) with custom parameters to keep multimaps (−k 100—very-sensitive-local—score-min “L,0,1.6”). The custom transcriptome consisted in the hg38 reference transcriptome with 14,968 HERVs transcriptional units compiled from RepeatMasker annotations (18). SAM outputs were converted to BAM files using SAMtools v1.4 (23). HERVs and genes expression was then calculated using Telescope (Bendall et al., 2019) and HTSeq 0.12.3 (24), respectively. Raw counts were then concatenated and normalized independently for each dataset using DESEQ2 v1.28.0 with variance stabilizing transformation (VST) (25).

### ATACseq Data

Significant peaks called from ATACseq data analysis were retrieved from the original paper (Corces et al., 2016). Briefly, peaks were called using MACS2 and filtered using a custom blacklist. A final set of 590,650 significant peaks were defined among a list of non-overlapping maximally significant 500 bp peaks ranked by their summit significance value. These significant peaks were re-annotated using HOMER with the command “annotatePeaks.pl” and two different references: Gencode v33 only and Gencode v33 with the previously used HERVs annotation. Regions containing significant peak around +/−1,000 or 3,000 bp of a HERV TSS were considered as active HERVs regions.

### Unsupervised Hierarchical Clustering

For the sorted cells, DESEQ2 VST normalized expression data were directly used for unsupervised hierarchical clustering. Cluster purity was used as an external validation criterion and was calculated by first creating a confusion matrix between assigned cluster number and annotated cell type before summing the maximum values from each row (i.e. assigned cluster) divided by the total number of samples.

A benchmark of distances (euclidean, maximum and pearson) and methods (ward.D2, single, complete, average and centroid) was performed to identify the optimal method leading to the best cluster purity using a pre-defined number of clusters according to the original annotation.

For the bulk datasets, DESEQ2 VST normalized expression data were independently calculated and further center-scaled for each dataset to correct the potential batch effect. Unsupervised hierarchical clustering was then performed using the average silhouette width and the Bayesian Information Criterion as internal validation markers.

### Differential ATAC-Count Analysis

For differential ATAC-count analysis, raw ATAC-seq count were retrieved from the original paper (Corces et al., 2016). Differential expression analysis between each AML populations (LSC, pHSC and Blasts) and their normal counterpart (HSC, GMP, LMPP and monocytes) was performed using DESEQ2, with cell type as a covariate. Differentially expressed regions surrounding a HERVs TSS (+/−20,000 bp) and with a FDR <5% were retained for the final plot. The rolling mean of 1,000 sequential regions, ordered by chromosome location, was then represented.

### HERVs, Genes and Copy Number Variation Correlations

HERVs located in previously defined active HERVs regions (so-called AHR+/−20,000 bp) were selected for correlation analysis. For each HERV, a list of surrounding genes located at +/−50,000 bp of their TSS was established. Pearson's correlations were calculated between the RNA expression of each HERVs and each of its surrounding gene, independently. P-values were corrected with the FDR method. Genes were then annotated using a published list of cancer-related genes from the Cancer Gene Census (20). The same list of HERVs was then used to perform correlations with CNV from the same cytoband. TCGA LAML CNV data were retrieved from the NCI GDC portal and used as is to calculate Pearson's correlations with HERVs from the same cytoband.

### Cancer Hallmark and Immune Signatures GSVA

For each hallmark of cancer (Hanahan and Weinberg, 2011), a unique gene signature was established based on The Molecular Signatures Database (MSigDb) Hallmark Gene Set Collection (26). When not available in MSigDb, hallmark signatures where established from Gene Ontology (GO) signatures, as previously described (27). Signature for the immune evasion hallmark was retrieved from Hubert et al. (28). Individual enrichment score were calculated from each patient by single sample gene-set variation analysis (ssGSVA), and scaled by study. The mean score for each cluster was then calculated and shown in a radar plot.

Immune signatures were obtained from Thorsson et al. (29) and calculated by ssGSVA for each sample. Unsupervised hierarchical clustering was then performed on study-scaled ssGSVA scores in each cluster.

### Differential HERVs Expression Analysis

Differential expression analysis was performed using DESEQ2 (25). HERVs and genes raw counts from all normal and AML datasets were merged and integrated into the same DESEQ object, using study (i.e. batch) as a covariate in the design formula. Differential expression analysis was performed for all the 4 independent bulk AML datasets and the sorted LSC and pHSC populations against each of the 42 normal tissues. Fold change were shrunk with the apeglm method (30). Features with a fold change superior to 4 (log 2FC >2) and a base mean of at least 1 normalized count per million were considered overexpressed.

### Biological Samples

Bone marrow samples were collected from AML patients at diagnosis at the Centre Hospitalier Lyon Sud in Lyon, France. Samples collection was approved from the institutional review board and ethics committee (20.01.31.72653-21/20_3) and after obtaining patients' written informed consent, in accordance with the Declaration of Helsinki. BMMCs were obtained by Ficoll density gradient centrifugation (Eurobio, FR, EU) and immediately cryoconserved in foetal bovine serum (FBS) with 10% dimethylsulfoxyde (DMSO).

### MILs Growth

BMMCs were rapidly thawed at 37° C. and put in culture in RPMI medium (Gibco, FR, EU) supplemented with 8% human AB-serum (Etablissement Français du Sang, FR, EU) and high doses (6,000 UL/mL) IL-2 (PROLEUKIN aldesleukine, Novartis Pharma, CH, EU) after a 2-hours resting. Plates were then incubated for 14 days, with medium replacement when needed.

TABLE 1

| cluster_id | | | |
|---|---|---|---|
| AML subtype | herv_id | coefficient | p, adj |
| 9 | ERV316A3_1q25.2b | 1.984674465 | 1.35E−21 |
| 9 | HERVH_12q15b | 1.830470574 | 8.59E−10 |
| 9 | HML2_10p14 | 1.7697463 | 5.22E−15 |
| 1 | HUERSP3_8q11.1 | 1.763328813 | 1.97E−08 |
| 7 | HERVH_4q22.3a | 1.69629645 | 6.19E−28 |
| 7 | HERVK11_4q13.2 | 1.68066449 | 1.49E−18 |
| 7 | HML2_10p14 | 1.6737451 | 1.46E−17 |
| 2 | HML3_Xq21.1a | 1.578269758 | 7.01E−20 |
| 9 | ERVLB4_13q12.12a | 1.575202077 | 9.67E−14 |
| 3 | HERVH_11q13.4a | 1.572097164 | 8.77E−25 |
| 9 | HERVIP10FH_Xp21.1 | 1.55149504 | 5.70E−17 |
| 9 | ERVLE_14q11.2d | 1.510681496 | 2.93E−15 |
| 5 | HERVH_10q23.31b | 1.504766105 | 1.07E−21 |
| 9 | HERVH_12q21.31 | 1.501291209 | 3.90E−07 |
| 3 | HERVE_11q13.4c | 1.491397809 | 1.83E−17 |
| 9 | HERVFH21_Xq21.1 | 1.485765776 | 3.10E−11 |
| 9 | HERV4_17p13.1 | 1.484104501 | 2.71E−08 |
| 9 | HERVW_14q11.2 | 1.483705144 | 1.72E−08 |
| 3 | HARLEQUIN_18p11.22a | 1.47754626 | 1.19E−14 |
| 9 | ERVLE_15q21.2 | 1.475648811 | 6.65E−14 |
| 5 | MER4_10q23.31b | 1.475516065 | 3.60E−23 |
| 9 | MER4_Xq21.1a | 1.45951717 | 6.29E−13 |
| 9 | HERVL_10q26.3b | 1.455695009 | 2.59E−15 |
| 5 | HERVW_3p11.1 | 1.446993977 | 2.79E−25 |
| 2 | HERVL_18p11.21a | 1.443269107 | 4.43E−17 |
| 5 | HERVH_10q23.31c | 1.440255766 | 6.65E−14 |
| 9 | HERVE_20p11.21b | 1.437083222 | 1.10E−05 |
| 7 | ERVLE_2q34e | 1.435016741 | 9.29E−16 |
| 7 | HERVH_1q24.2 | 1.433336362 | 1.28E−13 |
| 2 | HERVK11_4q13.2 | 1.428155115 | 7.54E−10 |
| 3 | HERVH_11q13.4b | 1.427635755 | 1.11E−14 |
| 3 | HERVFH21_1q23.1 | 1.426375673 | 1.05E−13 |
| 9 | MER41_7q11.21i | 1.423895135 | 0.016529581 |
| 8 | HERVE_1p36.12 | 1.421577776 | 5.79E−25 |
| 2 | MER4_10p12.33 | 1.41963502 | 9.04E−15 |
| 2 | HERVH_12q22 | 1.411090338 | 2.94E−15 |
| 9 | MER41_12p13.2 | 1.406294173 | 2.14E−08 |
| 9 | HERV3_19p12e | 1.40541287 | 3.60E−11 |
| 3 | HERVH_11p15.4b | 1.404089048 | 2.53E−15 |
| 9 | ERVLE_15q26.3b | 1.404044769 | 6.10E−13 |
| 3 | HERVE_7p22.1c | 1.40274314 | 1.01E−14 |
| 9 | HERVFH21_14q32.31a | 1.400753073 | 3.47E−12 |
| 9 | MER61_4p16.3 | 1.397771609 | 2.25E−07 |
| 7 | HERVL_21q21.1e | 1.397231683 | 5.80E−07 |
| 9 | ERV316A3_13q33.3b | 1.396928197 | 8.51E−04 |
| 3 | HML3_7q11.21 | 1.394028172 | 5.00E−15 |
| 3 | ERV316A3_2p25.1b | 1.392854604 | 4.63E−14 |
| 9 | HML6_19p12e | 1.386621228 | 7.65E−11 |
| 9 | HERVH_11p15.2 | 1.381811967 | 8.32E−14 |
| 3 | HERVS71_11p15.4 | 1.379471005 | 3.69E−15 |
| 3 | HERVL18_3p21.31a | 1.379048032 | 7.65E−11 |
| 9 | MER4_3q13.11 | 1.377356603 | 2.18E−05 |
| 3 | HERVE_11p15.4 | 1.37478784 | 6.18E−11 |
| 8 | ERVLB4_17q25.1 | 1.372237191 | 3.99E−37 |
| 3 | HERVH_7p22.1c | 1.371342414 | 8.13E−12 |
| 8 | HERVH_8q24.22a | 1.368729041 | 1.34E−08 |
| 9 | ERVLE_11p15.2b | 1.366701706 | 5.77E−07 |
| 2 | HERVS71_19p12b | 1.366504675 | 1.09E−04 |
| 9 | HERV9_6q25.1 | 1.365534682 | 9.31E−07 |
| 2 | ERV316A3_1q25.2b | 1.365116121 | 4.84E−09 |
| 4 | HERVS71_14q32.33 | 1.363294526 | 9.37E−17 |
| 9 | HERVFC1_7q11.21 | 1.36024375 | 2.35E−12 |
| 2 | HERVH_22q11.1 | 1.358279383 | 6.62E−09 |
| 5 | MER61_4q31.21a | 1.357568092 | 1.19E−16 |
| 2 | ERVLB4_8p21.1b | 1.357452637 | 3.07E−12 |
| 9 | MER4_11p15.4a | 1.354962252 | 6.08E−10 |
| 3 | HERVL18_3p21.31b | 1.354631416 | 1.53E−08 |
| 9 | HML2_8p23.1d | 1.353038047 | 5.86E−05 |
| 9 | PRIMA4_7q11.21b | 1.348039547 | 1.62E−06 |
| 8 | HERVL18_11q12.2 | 1.346806541 | 8.81E−22 |
| 9 | HERVS71_5q15 | 1.3460545 | 1.59E−14 |
| 9 | HERVH_14q32.31b | 1.342693408 | 4.98E−11 |
| 9 | ERVLE_1p36.12 | 1.341087833 | 1.16E−14 |
| 9 | ERVLE_11q13.1b | 1.340972357 | 1.91E−05 |
| 9 | HERVH_6p21.2b | 1.340844167 | 2.98E−06 |
| 9 | ERVLE_13q12.11 | 1.338280536 | 6.78E−11 |
| 9 | ERVLB4_22q11.21a | 1.338080954 | 2.91E−08 |
| 3 | HERVE_3q22.1b | 1.337929417 | 2.57E−05 |
| 3 | HERVE_17q11.2 | 1.337617999 | 3.96E−14 |
| 4 | HARLEQUIN_18p11.22b | 1.336621126 | 1.18E−09 |
| 2 | HML2_4p16.1b | 1.334762096 | 6.42E−14 |
| 9 | HERVH_7p14.3e | 1.33467427 | 1.23E−12 |
| 4 | HERV9_4q13.3 | 1.33325156 | 3.08E−11 |
| 9 | HML5_1q22 | 1.332373857 | 8.69E−12 |
| 8 | MER34B_1q23.3b | 1.33098623 | 2.96E−16 |
| 9 | ERV316A3_1p32.3a | 1.33050102 | 1.01E−04 |
| 9 | HERVI_8p23.1 | 1.32943344 | 1.31E−08 |
| 2 | HERVH_4p16.1d | 1.328409842 | 1.99E−14 |
| 2 | HERVL_3q25.1 | 1.328339625 | 1.03E−08 |
| 4 | HERVH_14q24.2a | 1.326193804 | 1.88E−09 |
| 3 | HERVS71_11q13.4 | 1.326025082 | 2.47E−08 |
| 2 | ERVLE_11q13.1b | 1.325830397 | 1.01E−06 |
| 3 | LTR46_19q13.43 | 1.325676639 | 1.29E−10 |
| 3 | HML6_3p21.31b | 1.320081183 | 1.81E−07 |
| 4 | HML1_Xq26.3 | 1.319627842 | 2.63E−07 |
| 9 | HUERSP2_1q32.1 | 1.317399844 | 2.44E−09 |
| 9 | PRIMA4_1q21.2c | 1.316314969 | 2.14E−08 |
| 9 | HUERSP3_11p15.1 | 1.315585225 | 3.05E−12 |
| 7 | HERVH_5p14.3a | 1.314646161 | 1.46E−09 |
| 5 | MER41_4q34.1a | 1.314248402 | 7.59E−10 |
| 1 | MER34B_1q23.3b | 1.313945842 | 4.80E−05 |
| 3 | HARLEQUIN_1q24.2 | 1.313266081 | 1.86E−09 |
| 4 | MER4_14q24.2c | 1.312479111 | 8.63E−12 |
| 9 | ERVLE_4p12e | 1.310026249 | 2.08E−08 |
| 9 | HERVL74_17q12 | 1.306772374 | 2.11E−10 |
| 5 | HERVL_21q21.1e | 1.305742517 | 1.64E−09 |
| 9 | HERVW_3p21.31 | 1.305392154 | 8.00E−07 |
| 7 | MER4_14q22.3a | 1.303361818 | 1.57E−06 |
| 5 | HERVH_6q12c | 1.303260802 | 8.40E−10 |
| 9 | ERV316A3_12p12.3c | 1.303129198 | 1.04E−10 |
| 1 | HERVH_10q23.31b | 1.302814023 | 0.047220074 |
| 9 | HERVH_19p12a | 1.301242421 | 2.33E−08 |
| 9 | HERVH_19p13.12 | 1.29757246 | 1.97E−08 |
| 8 | HERVL40_8q11.23b | 1.297490969 | 1.09E−06 |
| 6 | HERVH_2q24.1d | 1.294712441 | 8.68E−25 |
| 3 | HERVH_6p22.3a | 1.29461857 | 2.41E−13 |
| 3 | ERVLE_19q13.31a | 1.294239399 | 2.06E−08 |
| 9 | MER34B_1q23.3a | 1.293542561 | 1.72E−06 |
| 9 | MER4_1q21.2 | 1.292738002 | 2.09E−07 |
| 3 | HERVH_4q22.1c | 1.29090888 | 1.42E−07 |
| 8 | HARLEQUIN_11q12.2b | 1.290803519 | 2.89E−19 |
| 8 | HERVH_14q11.2b | 1.29067075 | 0.040164263 |
| 5 | HERVW_2q24.3 | 1.290293913 | 3.82E−15 |
| 5 | HERV4_15q26.1 | 1.29022567 | 1.76E−07 |
| 9 | MER4_5p12a | 1.289236895 | 5.49E−13 |
| 2 | HERVP71A_1p31.1a | 1.289073993 | 0.001113401 |
| 5 | HERVH_3q21.3d | 1.288871322 | 3.03E−13 |
| 8 | HERVH48_2q22.2 | 1.288528079 | 3.56E−14 |
| 6 | HERVFH21_1p36.31 | 1.288011396 | 8.88E−21 |
| 9 | HERVFRD_6p24.2 | 1.287980954 | 6.79E−11 |
| 9 | HML2_3p12.3 | 1.287902759 | 9.42E−11 |
| 8 | HERVE_2p14b | 1.28708828 | 5.72E−23 |
| 8 | MER4_4q25b | 1.28670212 | 1.34E−10 |
| 5 | HERVH_6q12f | 1.28535016 | 1.12E−14 |
| 7 | HERVL_12q13.11a | 1.284422469 | 8.67E−05 |
| 9 | HERVL_12p11.23 | 1.284349321 | 0.004663266 |
| 9 | LTR25_7q11.21 | 1.283978175 | 1.10E−07 |
| 8 | HUERSP3_19q13.42 | 1.282722503 | 5.46E−15 |
| 7 | HERVH_19p13.12 | 1.2822461 | 1.94E−08 |
| 8 | HERVL_11q12.2 | 1.28216679 | 3.89E−19 |
| 9 | HML3_6q21a | 1.282148451 | 1.59E−05 |
| 5 | HML6_19p12e | 1.281415813 | 1.51E−11 |
| 6 | ERVLB4_8p23.1f | 1.280627258 | 1.13E−07 |
| 9 | ERVL_Xq21.1a | 1.280565559 | 5.56E−09 |
| 9 | HERVH_14q24.2b | 1.280490226 | 8.88E−07 |
| 9 | HERVH_7p14.3d | 1.275341509 | 6.41E−12 |
| 9 | HERV4_19q13.41 | 1.275268773 | 4.78E−09 |
| 4 | HERVL_3q25.1 | 1.274899272 | 1.32E−09 |
| 9 | HERVIP10F_1q44 | 1.27410896 | 2.83E−06 |
| 9 | HML2_3q21.2 | 1.27332785 | 8.20E−06 |
| 3 | HERVH_3q22.1b | 1.27324086 | 8.01E−06 |
| 4 | ERVLE_Xq26.3h | 1.272551701 | 1.71E−05 |
| 2 | ERVLE_14q11.2d | 1.272201628 | 4.99E−10 |
| 8 | HERV4_11q21 | 1.271990445 | 2.85E−15 |
| 8 | HERVIP10FH_14q11.2 | 1.271570707 | 0.046762107 |
| 2 | HERVW_2q32.3 | 1.271016727 | 3.54E−06 |
| 7 | HML5_1q23.1 | 1.270950001 | 8.93E−06 |
| 7 | MER41_7q34b | 1.270308738 | 4.10E−08 |
| 4 | HML3_Xq21.1a | 1.268748036 | 7.23E−07 |
| 7 | HERVH_10q23.31c | 1.268553132 | 0.006145986 |
| 9 | HERVE_2q37.1 | 1.26830975 | 0.002901736 |
| 4 | ERVLE_4q21.1b | 1.268224136 | 1.86E−08 |
| 5 | MER4B_14q32.33d | 1.267918157 | 8.17E−20 |
| 9 | HERVIP10FH_14q23.1 | 1.267128726 | 2.01E−09 |
| 9 | HML5_7p14.1 | 1.267014459 | 9.08E−05 |
| 3 | HERVE_19q12 | 1.266891118 | 0.01040328 |
| 3 | HERVH_3p21.31e | 1.266821832 | 1.01E−11 |
| 5 | MER41_7q34b | 1.265287478 | 7.43E−14 |
| 9 | PRIMA41_19p12d | 1.264619965 | 2.46E−05 |
| 9 | HML6_19p13.2d | 1.263923457 | 2.45E−07 |
| 5 | PABLA_19q13.2 | 1.263168785 | 6.32E−19 |
| 3 | HERVE_20p11.21b | 1.262998215 | 2.84E−09 |
| 7 | HML2_11q12.1 | 1.262731071 | 2.96E−09 |
| 9 | ERVLB4_15q13.2c | 1.262260038 | 0.005042208 |
| 6 | ERVLB4_9q34.11 | 1.261153445 | 3.34E−09 |
| 5 | MER101_19q13.2c | 1.260748649 | 1.13E−12 |
| 2 | HERVFH21_Xq21.1 | 1.259293933 | 4.67E−05 |
| 7 | HERVH_8p23.1c | 1.258414265 | 0.002898852 |
| 9 | HERVL_6q14.1d | 1.258372211 | 7.93E−07 |
| 7 | MER4_10q23.31b | 1.257432716 | 2.67E−04 |
| 7 | HERVL_12q13.11b | 1.256925982 | 3.70E−06 |
| 7 | HERVH_10q23.31b | 1.256482067 | 0.002495365 |
| 6 | HERVH_8q24.21a | 1.255985277 | 6.05E−08 |
| 5 | MER41_7q11.21i | 1.255284081 | 4.14E−04 |
| 9 | HERVH_3p22.3b | 1.255085007 | 1.04E−04 |
| 7 | ERVLE_13q12.11 | 1.254654174 | 1.89E−09 |
| 7 | MER4_4q23b | 1.254539546 | 2.54E−16 |
| 9 | HUERSP1_Xp22.2 | 1.25437205 | 6.44E−06 |
| 9 | MER4_6q26b | 1.253858895 | 6.69E−04 |
| 4 | ERVLB4_2q13c | 1.253107681 | 9.96E−06 |
| 3 | HERVH_10p15.1 | 1.252980264 | 1.70E−05 |
| 2 | MER34B_10q25.3b | 1.252878433 | 9.39E−07 |
| 9 | ERVLB4_16p13.3a | 1.252846498 | 4.93E−07 |
| 2 | HERVS71_4p16.1b | 1.252713384 | 8.07E−09 |
| 9 | ERVLE_5q31.1c | 1.252603889 | 1.51E−06 |
| 3 | HERVH_2p14b | 1.251837134 | 8.30E−13 |
| 6 | MER61_2q35 | 1.251299612 | 2.50E−06 |
| 6 | ERV316A3_13q13.3c | 1.251216207 | 4.78E−04 |
| 7 | ERVLB4_4q12d | 1.251050147 | 0.016476965 |
| 6 | ERVLB4_8p23.1b | 1.250707147 | 3.78E−08 |
| 9 | HERVS71_19q13.12a | 1.25063148 | 1.11E−07 |
| 9 | HERVH_13q13.3a | 1.249417878 | 1.24E−06 |
| 3 | HERVH_19q13.31 | 1.249181403 | 5.84E−05 |
| 9 | HML2_4p16.1a | 1.249150489 | 3.84E−06 |
| 6 | ERV316A3_6q22.31g | 1.248262758 | 5.68E−08 |
| 8 | HERVE_2p14a | 1.248128398 | 1.87E−17 |
| 9 | MER34B_9p21.3b | 1.247466938 | 3.16E−06 |
| 9 | ERVLE_3p12.2h | 1.246954333 | 0.007103997 |
| 7 | HML6_2q14.3 | 1.246613349 | 0.004611159 |
| 9 | HML3_1p32.3 | 1.246518132 | 7.02E−06 |
| 9 | HERVH_19p13.2d | 1.246264627 | 1.74E−05 |
| 9 | LTR57_5q31.1 | 1.245622525 | 2.73E−07 |
| 9 | HERVL_Xq11.1a | 1.244967061 | 7.86E−10 |
| 1 | MER41_7q34b | 1.244750676 | 0.012798604 |
| 9 | ERVLE_4q24g | 1.244648856 | 0.00104322 |
| 9 | ERVLE_2q24.3b | 1.244552454 | 3.72E−04 |
| 7 | HML3_17p13.1 | 1.244352302 | 1.40E−07 |
| 9 | ERVLE_3p12.2g | 1.24334677 | 0.019251299 |
| 9 | ERV316A3_18q21.1b | 1.242924845 | 6.17E−06 |
| 9 | HML2_20q11.22 | 1.241314615 | 5.50E−04 |
| 9 | MER101_16p12.2a | 1.241188684 | 9.75E−05 |
| 3 | HERVH_4q21.21a | 1.240906899 | 6.92E−04 |
| 5 | HML2_22q11.23 | 1.240613744 | 9.82E−07 |
| 5 | MER61_4p16.3 | 1.24020626 | 1.84E−07 |
| 7 | HERVK11_8p23.1c | 1.240163347 | 0.002117351 |
| 8 | HERVL_9q21.33b | 1.240156852 | 2.52E−12 |
| 6 | HERVIP10FH_6q25.3 | 1.239886375 | 5.82E−06 |
| 6 | HERVH_4q22.1c | 1.239825629 | 3.49E−06 |
| 1 | HERVL18_11q12.2 | 1.239617384 | 0.041396592 |
| 3 | HERVH_2p16.1e | 1.239226976 | 1.19E−06 |
| 5 | HERVW_1q32.1 | 1.239096957 | 1.45E−05 |
| 2 | HERVIP10F_11p14.3 | 1.238919065 | 2.48E−05 |
| 8 | PRIMA41_13q13.1 | 1.238613137 | 2.78E−07 |
| 8 | HERVH_20p13c | 1.237741806 | 5.50E−10 |
| 9 | HARLEQUIN_7q22.1 | 1.237720154 | 0.009817645 |
| 5 | MER41_9p13.3a | 1.23730697 | 1.03E−08 |
| 5 | HML2_11q12.1 | 1.237301862 | 1.26E−14 |
| 5 | HERVL_1p13.3b | 1.236994261 | 1.31E−08 |
| 2 | HERVL40_13q32.1 | 1.236192892 | 8.22E−06 |
| 9 | HERVH_3q21.3b | 1.235899607 | 7.92E−04 |
| 7 | MER4_Xq21.1a | 1.235838372 | 4.20E−06 |
| 2 | HERVW_19p12b | 1.235270954 | 8.82E−05 |
| 7 | HARLEQUIN_1q32.1 | 1.235247772 | 2.49E−08 |
| 5 | HUERSP3B_1q21.3 | 1.234790315 | 0.007463733 |
| 8 | HERVL_12q14.2a | 1.234672184 | 4.14E−05 |
| 9 | ERV316A3_14q24.2f | 1.234289217 | 0.006906994 |
| 9 | HML3_17p13.1 | 1.234253266 | 4.63E−06 |
| 9 | HERV9_13q34 | 1.233932591 | 1.40E−04 |
| 9 | HERVH48_19p12b | 1.23272881 | 1.22E−04 |
| 6 | HERVL_11q12.1d | 1.232385794 | 7.89E−10 |
| 7 | ERVLE_1p22.3c | 1.232301135 | 5.59E−05 |
| 4 | HERV3_4q13.3b | 1.23215349 | 1.11E−08 |
| 7 | HUERSP3_1q44 | 1.232152863 | 1.63E−10 |
| 9 | MER4_11q14.1 | 1.232076735 | 5.94E−04 |
| 5 | HARLEQUIN_2q32.1 | 1.231924278 | 1.79E−13 |
| 5 | MER41_12p12.1b | 1.231910027 | 1.78E−12 |
| 6 | HERVH48_11p15.4 | 1.231840603 | 2.21E−07 |
| 6 | ERVLE_4q21.23c | 1.23099081 | 9.04E−09 |
| 2 | ERVLB4_2q32.3 | 1.230953829 | 5.50E−04 |
| 9 | HERVH_Xq26.2 | 1.230878194 | 0.007454823 |
| 8 | MER34B_19p13.11 | 1.230294482 | 3.89E−10 |
| 9 | HERVEA_19p13.11 | 1.230058789 | 7.29E−07 |
| 9 | MER41_Xq11.1c | 1.230028602 | 0.005895606 |
| 5 | ERVLE_2q34e | 1.229893575 | 1.11E−09 |
| 3 | ERVLB4_1p34.2b | 1.229788979 | 3.25E−05 |
| 5 | ERVLE_1p22.3c | 1.228810711 | 1.20E−09 |
| 6 | MER34B_19p13.11 | 1.22805169 | 1.73E−09 |
| 3 | MER4_14q24.2c | 1.22790909 | 1.89E−05 |
| 5 | HUERSP2_Yq11.223 | 1.227817339 | 0.001212901 |
| 9 | HERVH_1q42.2b | 1.22768306 | 5.79E−04 |
| 9 | ERV316A3_10q22.1a | 1.227595055 | 4.08E−07 |
| 3 | HUERSP3_9p23 | 1.22663235 | 1.62E−06 |
| 9 | HERV3_19p12b | 1.226395865 | 3.15E−04 |
| 9 | ERVLE_1p36.11b | 1.226080684 | 2.04E−04 |
| 6 | HERVH_19q13.42b | 1.225848409 | 7.49E−05 |
| 5 | HERVL66_17_GL000205v2_random(a) | 1.225382902 | 9.62E−11 |
| 2 | HML5_19p12a | 1.225014366 | 0.001377693 |
| 9 | ERVLB4_19q13.12 | 1.224970157 | 2.59E−06 |
| 9 | HERVK11_1p13.3 | 1.224773501 | 5.05E−04 |
| 5 | HML1_19p12c | 1.224350181 | 3.44E−14 |
| 5 | HML1_1q32.1 | 1.224020746 | 0.001659544 |
| 6 | ERVL_1p36.22a | 1.223285204 | 3.29E−13 |
| 2 | HERVL_12q13.13 | 1.222862685 | 1.95E−05 |
| 5 | HUERSP2_2q24.2 | 1.22250743 | 1.02E−20 |
| 5 | LTR25_Yq11.223d | 1.222043899 | 1.78E−04 |
| 7 | HERVE_8p23.1h | 1.221783984 | 0.006728393 |
| 9 | MER4_9p13.3a | 1.221120368 | 8.45E−07 |
| 6 | ERVLE_12p13.31d | 1.220801438 | 5.30E−06 |
| 4 | ERVLB4_2q32.3 | 1.220600388 | 3.41E−05 |
| 6 | HUERSP1_11p15.4a | 1.22056123 | 2.36E−06 |
| 8 | HERVH_12p13.31e | 1.219627106 | 1.10E−04 |
| 8 | MER61_12q15a | 1.219526942 | 0.011868077 |
| 9 | HERVL40_5q12.3 | 1.219500594 | 0.001675875 |
| 4 | HERV9_7q21.11 | 1.219368813 | 0.017218684 |
| 8 | HERVH_10q23.31d | 1.219260847 | 5.87E−04 |
| 9 | ERVLE_13q14.13 | 1.218847368 | 2.55E−06 |
| 5 | HERVH_21q22.3 | 1.218484891 | 1.61E−15 |
| 9 | HERVH_19q13.12a | 1.218416013 | 0.001117847 |
| 9 | LTR25_4q34.1a | 1.218395157 | 2.19E−05 |
| 1 | HARLEQUIN_1q32.1 | 1.218257944 | 0.004002809 |
| 7 | HERVH_19q13.12b | 1.218083523 | 7.21E−04 |
| 9 | HERV4_19q13.43b | 1.218014814 | 0.001741368 |
| 9 | HML5_12q23.1 | 1.217067234 | 4.21E−08 |
| 9 | HERV3_7q34a | 1.216916226 | 8.89E−05 |
| 7 | HERVH_5q13.3b | 1.216903718 | 0.03332367 |
| 9 | HUERSP3_19q11 | 1.216862381 | 4.74E−04 |
| 9 | ERVLB4_Xq11.1b | 1.216250437 | 0.007103997 |
| 3 | HERVH_2p24.3a | 1.21620497 | 1.94E−04 |
| 7 | HERVEA_12p13.31 | 1.216036416 | 3.05E−04 |
| 9 | ERVL_5q11.2 | 1.215349028 | 1.79E−05 |
| 5 | HERV3_Yq11.223b | 1.215156033 | 0.003349685 |
| 2 | HML3_6q21b | 1.214256568 | 0.008314531 |
| 6 | HERVE_3p26.3 | 1.214093808 | 4.10E−07 |
| 3 | HML2_11p15.4a | 1.214001976 | 0.00153081 |
| 9 | HERVIP10F_19p13.11 | 1.21355669 | 2.06E−06 |
| 2 | HERVE_9q34.3 | 1.21346214 | 2.02E−06 |
| 6 | HERVW_6q14.3a | 1.213104427 | 5.80E−07 |
| 2 | HERVH_12q21.31 | 1.21128445 | 0.044012277 |
| 2 | ERVL_11p14.3 | 1.210936019 | 6.37E−08 |
| 5 | HARLEQUIN_1q32.1 | 1.210913953 | 3.18E−13 |
| 5 | PRIMA4_14q22.1 | 1.210742527 | 0.007004588 |
| 5 | HERVL40_Xq22.2 | 1.210561445 | 1.59E−05 |
| 8 | HERVH_1q42.13c | 1.210430217 | 9.46E−07 |
| 5 | HML2_7q34 | 1.210364597 | 1.15E−13 |
| 5 | HML5_1q23.1 | 1.210330693 | 4.12E−07 |
| 5 | HUERSP1_11p15.4b | 1.20987156 | 5.67E−11 |
| 9 | HERVS71_7p14.3 | 1.209856208 | 2.34E−09 |
| 6 | HUERSP3_19q13.42 | 1.209759255 | 6.41E−04 |
| 5 | HERVH_1q32.1a | 1.209595392 | 1.42E−04 |
| 6 | HML5_12p13.31 | 1.209558472 | 0.00659921 |
| 9 | HERVH_4p16.1a | 1.209252273 | 4.24E−04 |
| 6 | ERVLE_19q13.31a | 1.208934747 | 7.41E−05 |
| 5 | HERV3_19p12e | 1.208856686 | 2.85E−09 |
| 8 | HERVE_2q37.1 | 1.208775988 | 3.11E−04 |
| 6 | HERVK11_1q23.3b | 1.208405828 | 3.21E−04 |
| 5 | HERVIP10FH_5q35.1 | 1.208173975 | 1.21E−10 |
| 9 | HML6_19q13.41f | 1.207712964 | 1.17E−04 |
| 5 | HML3_1q32.1b | 1.207242185 | 0.007666987 |
| 5 | LTR19_4q35.1 | 1.207169604 | 0.001559934 |
| 9 | HERVL66_19p12a | 1.206880767 | 4.99E−04 |
| 9 | HML3_Xp11.21 | 1.206636099 | 0.007486007 |
| 6 | ERV316A3_17q21.33c | 1.206412052 | 6.37E−10 |
| 9 | HML6_19q13.41c | 1.205959393 | 1.66E−05 |
| 3 | HERVL74_2q11.2 | 1.205879327 | 1.19E−04 |
| 6 | MER4B_Yq11.221a | 1.205341267 | 0.0016244 |
| 9 | PRIMA4_10p12.1 | 1.205307448 | 3.91E−05 |
| 3 | HML3_1q32.1b | 1.205298469 | 0.004604711 |
| 5 | PRIMA4_7q11.21b | 1.205149674 | 3.33E−06 |
| 5 | HERVL_4p13a | 1.205141593 | 1.12E−04 |
| 7 | HERVW_14q11.2 | 1.205072027 | 0.016829167 |
| 9 | HERVH_Xq13.1b | 1.204896687 | 5.97E−06 |
| 5 | ERVLE_8q22.3b | 1.204808483 | 4.78E−08 |
| 7 | PABLA_19q13.2 | 1.204661888 | 1.43E−06 |
| 9 | MER101_3p12.3 | 1.204203892 | 1.17E−05 |
| 9 | MER4_15q21.2a | 1.203925746 | 0.006684963 |
| 5 | HERVL_1p22.2d | 1.203669927 | 1.84E−15 |
| 2 | HERVH_6p21.2b | 1.203532713 | 0.002772962 |
| 9 | HML3_19p13.2d | 1.203443754 | 7.01E−06 |
| 9 | HERVH_2p13.3a | 1.20281026 | 8.39E−07 |
| 5 | HERVW_12p13.31b | 1.20247563 | 3.17E−04 |
| 5 | MER101_8p21.3 | 1.201281791 | 2.91E−10 |
| 5 | LTR25_Xq22.2b | 1.201009799 | 2.78E−05 |
| 9 | HML3_16p13.3 | 1.200935435 | 3.21E−07 |
| 7 | HUERSP3_10q23.33b | 1.20070508 | 0.004806991 |
| 6 | HERVL_16q24.1 | 1.200623336 | 1.05E−04 |
| 5 | ERVLB4_1p22.3a | 1.200611042 | 6.36E−05 |
| 5 | HERVH_19p13.2a | 1.200572387 | 1.52E−11 |
| 5 | ERV316A3_3q27.3e | 1.200310527 | 2.07E−15 |
| 7 | LTR25_4q34.1a | 1.200136321 | 4.82E−06 |
| 2 | HERVW_14q11.2 | 1.199833007 | 0.015918519 |
| 4 | ERV316A3_5q31.3c | 1.199425468 | 1.39E−09 |
| 3 | HERVL_11q12.1c | 1.199403597 | 4.40E−04 |
| 8 | HERV3_3q21.3 | 1.199094504 | 3.89E−06 |
| 9 | HERVL_17q12a | 1.198811961 | 1.12E−05 |
| 7 | HUERSP3_2p13.3 | 1.198809267 | 5.90E−06 |
| 6 | HERVL_1p12 | 1.198720125 | 4.68E−06 |
| 9 | HML1_19p13.2 | 1.198637374 | 3.99E−05 |
| 5 | ERVLB4_1p36.11 | 1.19847401 | 1.60E−11 |
| 9 | HERVH_20p12.1c | 1.198148317 | 0.008794617 |
| 5 | ERVL_22q12.3b | 1.197979641 | 1.17E−15 |
| 5 | HERVH_4q22.3a | 1.197977054 | 1.01E−05 |
| 7 | LTR25_2p13.3 | 1.197800018 | 1.09E−05 |
| 5 | HERV30_6q22.31 | 1.197774364 | 5.82E−04 |
| 9 | HML3_19p12b | 1.197419068 | 6.09E−05 |
| 3 | ERVLE_1q32.2d | 1.196984002 | 8.72E−05 |
| 9 | ERVLE_5q31.1b | 1.196928821 | 9.33E−05 |
| 9 | HERVK11D_2q11.2 | 1.19678883 | 0.006127419 |
| 6 | HARLEQUIN_18p11.22a | 1.196738284 | 5.09E−05 |
| 7 | ERVLB4_14q31.3b | 1.195768113 | 1.06E−05 |
| 4 | HERVH_7p21.1b | 1.195709876 | 0.004428221 |
| 6 | HML3_6q13 | 1.195467382 | 1.16E−05 |
| 6 | HERVIP10FH_4p15.32 | 1.195370086 | 1.35E−04 |
| 5 | HERVW_5p14.3 | 1.195326052 | 1.07E−14 |
| 5 | HERVEA_21q21.1 | 1.195102877 | 6.41E−10 |
| 9 | HERV4_19q13.43a | 1.194935634 | 0.001509133 |
| 9 | MER61_3q28 | 1.194800822 | 0.009508447 |
| 7 | HERVW_5p14.3 | 1.194744138 | 2.34E−05 |
| 2 | HERVH_14q32.31b | 1.19457128 | 3.23E−05 |
| 3 | HERVH_19q13.2a | 1.194515955 | 4.93E−06 |
| 3 | HERV9_7q21.13 | 1.1943829 | 0.004230127 |
| 5 | HERV3_11p15.5 | 1.194241839 | 4.23E−16 |
| 8 | ERVLE_8q12.3f | 1.193846853 | 2.34E−04 |
| 6 | HERVE_20p11.21b | 1.193747619 | 1.05E−07 |
| 6 | HERVH_17q25.1a | 1.19361828 | 1.02E−09 |
| 6 | HERVH_2q24.1e | 1.193150487 | 9.48E−06 |
| 9 | ERVLE_1p35.1c | 1.193088889 | 0.00139802 |
| 5 | ERVLE_11q14.2a | 1.192950399 | 1.02E−14 |
| 7 | HUERSP3_10q23.33a | 1.192937359 | 0.003219409 |
| 9 | HARLEQUIN_1q42.13c | 1.192871001 | 7.28E−04 |
| 5 | HERVL_11q12.1c | 1.192683418 | 1.04E−10 |
| 8 | ERV316A3_8q24.21c | 1.192051988 | 4.66E−08 |
| 2 | MER4_11q21e | 1.192041111 | 1.12E−04 |
| 6 | HERVL_7q32.1 | 1.192005531 | 8.21E−06 |
| 5 | ERV316A3_5q35.3a | 1.191878449 | 3.74E−06 |
| 9 | HML3_2p24.1 | 1.191849502 | 8.51E−04 |
| 7 | HUERSP2_1q32.1 | 1.191587454 | 0.039220224 |
| 9 | MER101_2q35a | 1.191503129 | 6.32E−04 |
| 5 | ERVLB4_14q31.3b | 1.191479662 | 1.47E−07 |
| 6 | HERVW_19p12a | 1.191449734 | 2.72E−06 |
| 9 | HERVFH21_19p12 | 1.191332234 | 0.027520763 |
| 6 | ERV316A3_17q21.33a | 1.191299587 | 9.77E−09 |
| 5 | HML1_6q22.32 | 1.191207595 | 5.41E−11 |
| 9 | HML5_5p13.2 | 1.191144615 | 0.030270352 |
| 3 | HERVH_19q13.42b | 1.190707027 | 4.06E−04 |
| 5 | ERVLB4_20p12.3c | 1.190676028 | 4.64E−05 |
| 6 | HML3_9q34.13 | 1.190627583 | 2.32E−07 |
| 5 | HML3_6p22.1 | 1.190559555 | 3.36E−05 |
| 7 | ERVL_Xq21.1a | 1.190242819 | 9.83E−05 |
| 6 | HERVH_2p16.1e | 1.190177832 | 0.02109689 |
| 5 | HERVIP10FH_1q23.1 | 1.190156225 | 1.41E−10 |
| 7 | HUERSP3_19q11 | 1.189843225 | 2.87E−04 |
| 6 | HERVW_2q22.2 | 1.189780136 | 0.004905827 |
| 4 | HUERSP3_9p23 | 1.189542019 | 2.02E−04 |
| 7 | HERVIP10F_1q44 | 1.189211147 | 9.67E−04 |
| 2 | HERVIP10FH_Xq24 | 1.189190463 | 0.044791836 |
| 1 | HERVK11_2q14.2 | 1.189056205 | 0.002591635 |
| 6 | ERVLE_2q24.3b | 1.188478433 | 2.49E−07 |
| 9 | PRIMA4_18q23b | 1.187948212 | 0.001626443 |
| 9 | HML6_12q24.12a | 1.187873401 | 1.72E−04 |
| 9 | HERV3_11q13.3 | 1.187791684 | 1.19E−05 |
| 9 | HERVFRD_9p21.3b | 1.187752317 | 1.76E−05 |
| 8 | MER4_4q25a | 1.187552544 | 2.90E−05 |
| 7 | HERVH_4p16.3b | 1.187490814 | 0.001428564 |
| 6 | HERVH_1q25.3 | 1.187429263 | 2.06E−06 |
| 9 | HERVL40_1q44 | 1.187369738 | 0.005327715 |
| 6 | HERVL_9q21.11a | 1.187182624 | 0.002151137 |
| 7 | HERVL66_4p16.3 | 1.186984958 | 8.90E−04 |
| 9 | HERVH_17q25.3 | 1.186835979 | 0.004633708 |
| 2 | HERVH_12q13.2b | 1.186354545 | 1.06E−07 |
| 6 | HERVH_19q13.32e | 1.186347308 | 4.99E−04 |
| 5 | ERVLE_19q13.31c | 1.186295169 | 2.92E−08 |
| 5 | MER41_19q13.2a | 1.185756382 | 6.54E−06 |
| 4 | MER4_10p12.33 | 1.185536422 | 0.004071347 |
| 5 | HERVH_Xq22.2c | 1.185091452 | 7.81E−08 |
| 9 | MER4B_11q12.1 | 1.185044389 | 0.011118112 |
| 6 | ERVLB4_1p36.22 | 1.185039978 | 4.99E−08 |
| 5 | HERVH_5q13.3b | 1.185016035 | 1.80E−04 |
| 5 | MER41_12p13.2 | 1.184314054 | 8.63E−06 |
| 2 | HERVH_Xq22.2b | 1.184280544 | 0.005449425 |
| 5 | LTR19_12p13.31 | 1.183817366 | 0.001315682 |
| 6 | HERVEA_3p24.3b | 1.183645859 | 0.001842632 |
| 7 | HERVL18_6p22.1a | 1.183461479 | 1.23E−06 |
| 9 | ERV316A3_18q11.2c | 1.183406805 | 0.002257915 |
| 9 | HERVH_Xq13.1a | 1.18328653 | 1.25E−04 |
| 5 | ERV316A3_22q13.33a | 1.18316243 | 1.98E−10 |
| 6 | HERVL40_2q37.3 | 1.182742159 | 3.81E−04 |
| 5 | HERVKC4_19q13.41 | 1.182695981 | 1.35E−06 |
| 5 | HERVL40_2q32.2 | 1.182650624 | 3.25E−12 |
| 9 | HERVH_9p13.3b | 1.182614874 | 0.018733398 |
| 9 | MER101_6q27b | 1.182373946 | 0.00288887 |
| 5 | ERVLB4_2q13c | 1.182177839 | 5.96E−04 |
| 2 | ERV316A3_17q21.33a | 1.182139066 | 0.001406082 |
| 5 | HERVH_1p22.1 | 1.181914288 | 3.46E−13 |
| 4 | HML2_4p16.1b | 1.181781028 | 2.09E−05 |
| 5 | MER41_Yq11.221a | 1.181779747 | 3.17E−05 |
| 5 | ERVLE_19q13.2b | 1.181700856 | 6.92E−07 |
| 5 | HERVH_14q22.1a | 1.181583361 | 5.29E−07 |
| 5 | HERVP71A_1q44b | 1.181551825 | 1.10E−08 |
| 9 | HML6_Xp11.22 | 1.181338439 | 7.30E−04 |
| 5 | HML2_3q21.2 | 1.18109713 | 2.23E−08 |
| 5 | ERV316A3_6p22.1m | 1.180720076 | 7.52E−08 |
| 9 | HML3_16p13.13 | 1.180511 | 0.008891867 |
| 5 | HERVH_4p16.3b | 1.180360761 | 4.75E−06 |
| 9 | MER4_2p21 | 1.180254061 | 0.010562959 |
| 5 | HERVL_6q14.1f | 1.18024052 | 4.34E−09 |
| 9 | HERVE_13q14.13b | 1.180070108 | 5.12E−08 |
| 9 | HERVP71A_6p22.1 | 1.179927893 | 9.57E−04 |
| 5 | MER41_6q21b | 1.179874641 | 1.99E−05 |
| 5 | MER41_12q13.11 | 1.179415615 | 3.29E−10 |
| 7 | HERVFRD_6p24.2 | 1.179304683 | 3.86E−06 |
| 2 | HERVFH21_14q32.31a | 1.179072962 | 7.73E−04 |
| 6 | ERVLE_11q23.3c | 1.178887806 | 7.17E−04 |
| 9 | HARLEQUIN_17q21.31 | 1.178572788 | 3.09E−06 |
| 5 | HUERSP3B_11p12 | 1.178549024 | 8.52E−10 |
| 7 | HERVH_10q26.12 | 1.178376656 | 0.028786444 |
| 6 | ERV316A3_8q11.21a | 1.178132345 | 3.36E−04 |
| 9 | MER101_12p13.31b | 1.178073389 | 6.53E−07 |
| 6 | ERVLE_1q32.2d | 1.177980263 | 0.008127938 |
| 9 | HERVH48_21q22.3 | 1.177859149 | 0.033472512 |
| 5 | HML3_Yq11.221b | 1.177628599 | 0.049426477 |
| 5 | HERVW_4p16.3 | 1.17736024 | 8.55E−06 |
| 9 | HML3_3q22.1 | 1.177311204 | 0.007710946 |
| 4 | HERVL_18p11.21a | 1.177223352 | 0.022043274 |
| 5 | LTR25_3p22.1 | 1.177162617 | 2.60E−07 |
| 6 | HML5_14q32.33b | 1.177126738 | 7.17E−04 |
| 7 | HERVH_Xq13.1a | 1.176864479 | 1.71E−06 |
| 6 | LTR46_5q33.1 | 1.1765001 | 4.42E−09 |
| 9 | ERV316A3_2q37.1a | 1.176350939 | 0.008911912 |
| 5 | HERVL66_4p16.3 | 1.17624663 | 1.54E−08 |
| 2 | MER61_10q21.3 | 1.176063354 | 0.008578693 |
| 5 | HERVP71A_12p13.31a | 1.175888628 | 5.92E−06 |
| 5 | HML3_6q21a | 1.175628771 | 1.37E−07 |
| 5 | HERVI_16p13.13 | 1.175623469 | 8.59E−08 |
| 9 | MER4_19p12a | 1.175439875 | 0.007839783 |
| 4 | HUERSP3_10q23.33a | 1.175384688 | 0.002078405 |
| 7 | HERVFH21_1p34.3 | 1.175192203 | 2.20E−05 |
| 9 | LTR19_19p13.2b | 1.175149518 | 0.026712041 |
| 9 | MER41_1q41a | 1.174922484 | 1.54E−04 |
| 4 | MER61_10q21.3 | 1.174875434 | 2.92E−05 |
| 6 | HERVFRD_18p11.23 | 1.174817065 | 0.010589834 |
| 6 | HERVH_22q11.1 | 1.174725451 | 0.010542802 |
| 4 | ERVLE_8q12.3f | 1.174629262 | 0.012042919 |
| 2 | HERVH_Xq22.2c | 1.174541571 | 0.005758205 |
| 3 | HERVIP10F_14q11.2c | 1.17449734 | 1.05E−04 |
| 5 | HUERSP3_Yq11.223a | 1.17444328 | 6.67E−04 |
| 5 | HERV3_14q32.33 | 1.174377186 | 2.16E−15 |
| 9 | HARLEQUIN_5q14.2 | 1.17413482 | 9.80E−04 |
| 8 | MER41_1q44a | 1.17411634 | 8.14E−12 |
| 8 | MER61_2q35 | 1.173975489 | 0.003156313 |
| 7 | HERVH_14q22.1a | 1.173775303 | 0.034208731 |
| 9 | LTR25_2q32.3a | 1.173585151 | 4.53E−04 |
| 3 | HML5_6p22.2 | 1.173582091 | 4.01E−04 |
| 6 | HUERSP2_Yq11.221 | 1.173430328 | 0.001607429 |
| 9 | HERVH_14q32.31c | 1.173185259 | 0.00901883 |
| 7 | HERV3_4q13.3b | 1.172888381 | 0.001758154 |
| 2 | MER4_11q21d | 1.172550495 | 0.012579394 |
| 7 | HERVH_Xp22.33 | 1.172549716 | 0.003971846 |
| 6 | HERVL_4p13a | 1.17225267 | 0.008048089 |
| 9 | HERVW_6q27b | 1.172030419 | 0.007031813 |
| 9 | HERV9_1p31.1 | 1.172016301 | 0.00161322 |
| 5 | ERV316A3_5q31.3c | 1.171798351 | 1.26E−05 |
| 6 | HERVP71A_12p13.33b | 1.171710521 | 5.08E−05 |
| 7 | HARLEQUIN_2q32.1 | 1.171479489 | 2.06E−04 |
| 7 | HERV9_7p14.1 | 1.171435631 | 0.039512065 |
| 7 | HERVS71_5q15 | 1.171316415 | 0.003769573 |
| 9 | MER4_3q29b | 1.171224841 | 0.009236659 |
| 5 | HERVL_12q13.11b | 1.171186548 | 3.11E−07 |
| 5 | HERV4_4q12 | 1.170743176 | 6.31E−11 |
| 7 | HERVIP10F_19p13.2b | 1.170676603 | 0.001230546 |
| 7 | ERVLE_1q24.2 | 1.170542729 | 7.73E−04 |
| 9 | HML5_13q33.1 | 1.170324179 | 7.12E−05 |
| 9 | ERVLE_12q14.3a | 1.170223684 | 1.63E−04 |
| 9 | HERVH_3q21.3c | 1.169961217 | 0.048687045 |
| 9 | HERVL_1q42.13 | 1.169572342 | 2.96E−04 |
| 8 | HERVH_8q22.1a | 1.169459019 | 8.97E−11 |
| 5 | ERVLE_6q22.31b | 1.169373767 | 2.90E−04 |
| 4 | HML5_1q42.12 | 1.168950659 | 7.20E−05 |
| 5 | HERVH_Xq22.2b | 1.168879448 | 1.18E−06 |
| 3 | HML6_7p13 | 1.16841095 | 3.79E−04 |
| 2 | HARLEQUIN_2q32.1 | 1.168190061 | 0.002770261 |
| 5 | HERV4_19q13.43b | 1.167742326 | 1.14E−06 |
| 4 | HERVH_4p16.1d | 1.167553023 | 0.001407919 |
| 3 | ERVLB4_2q32.3 | 1.167475812 | 0.00596115 |
| 9 | HERV3_11p15.5 | 1.167470546 | 0.016211156 |
| 5 | MER41_22q12.3e | 1.167445121 | 4.86E−11 |
| 9 | HERVW_5p13.2a | 1.167215869 | 0.009677976 |
| 7 | HERVW_3q26.32 | 1.16711966 | 0.023564988 |
| 9 | ERVLB4_1p36.11 | 1.166795874 | 0.011036736 |
| 3 | HERVH_14q24.2a | 1.16636184 | 0.002075377 |
| 7 | HERVIP10FH_19p12c | 1.166347677 | 4.26E−04 |
| 9 | HERVH48_8q24.13 | 1.166285857 | 0.029720358 |
| 7 | HML6_19p13.2d | 1.166093572 | 0.001972972 |
| 5 | HERVIP10FH_19p12c | 1.165456053 | 3.86E−06 |
| 9 | HERVS71_19q13.12b | 1.16494045 | 9.69E−05 |
| 6 | MER4_14q32.2 | 1.164304578 | 2.38E−04 |
| 6 | HERV9_7q21.13 | 1.1642146 | 0.040744323 |
| 9 | HML2_3q12.3 | 1.164034236 | 0.014081309 |
| 5 | ERVLE_1p13.3b | 1.163741064 | 6.51E−08 |
| 6 | MER4_4q25a | 1.163718623 | 0.001244377 |
| 2 | HERVL18_7p14.2 | 1.163323879 | 0.002577172 |
| 5 | HARLEQUIN_7q22.1 | 1.163196293 | 2.61E−07 |
| 2 | ERV316A3_17q21.33c | 1.162940686 | 0.023168707 |
| 5 | LTR25_12p11.22b | 1.162597606 | 4.50E−08 |
| 7 | HUERSP3_10q23.33c | 1.161611363 | 0.011530271 |
| 5 | LTR25_12p11.22a | 1.161593968 | 1.81E−05 |
| 5 | HERVFRD_2p11.2 | 1.161399904 | 6.94E−08 |
| 5 | HUERSP1_19q13.2a | 1.161191497 | 2.49E−08 |
| 3 | HERVH_19q13.32e | 1.161181317 | 1.21E−04 |
| 9 | HERVL18_6p22.1a | 1.161180769 | 0.002475715 |
| 5 | HERVW_6q27b | 1.161168921 | 1.12E−08 |
| 7 | HARLEQUIN_6p25.2 | 1.161100555 | 0.038370621 |
| 9 | LTR25_3p22.1 | 1.160342712 | 0.048957568 |
| 7 | HERVH_12q13.2b | 1.160234157 | 1.90E−05 |
| 7 | HML5_Xq11.1 | 1.159737928 | 0.015212171 |
| 5 | HERVW_Yq11.222b | 1.159674789 | 0.04574691 |
| 5 | HERVL18_2q32.1 | 1.159599126 | 9.66E−09 |
| 6 | HERVE_17q11.2 | 1.159376709 | 0.026057121 |
| 5 | HERVH_12p11.22a | 1.159244877 | 1.70E−07 |
| 9 | HML3_6q21b | 1.159084383 | 0.046912918 |
| 7 | HERVEA_21q21.1 | 1.158861949 | 0.015317321 |
| 7 | ERVLE_10q25.3a | 1.158795772 | 0.006539016 |
| 5 | LTR57_14q22.1 | 1.15871672 | 9.80E−09 |
| 6 | HERVS71_19p13.3 | 1.158325037 | 4.74E−06 |
| 5 | MER41_6q22.31b | 1.157966644 | 0.034615346 |
| 5 | MER41_4p15.2 | 1.157395981 | 1.01E−09 |
| 9 | HML2_12q24.33 | 1.157370339 | 0.001448347 |
| 5 | ERVLE_10q25.3a | 1.157237141 | 2.39E−04 |
| 2 | ERVLB4_22q11.21a | 1.157167176 | 0.00259857 |
| 6 | HERVL40_8q11.23b | 1.157096169 | 0.021963676 |
| 9 | LTR19_6q25.3a | 1.156948019 | 0.036375147 |
| 6 | ERVLE_8q22.3b | 1.156697619 | 3.41E−04 |
| 9 | HERV3_14q32.33 | 1.15660127 | 1.66E−04 |
| 5 | HERVL40_9q34.3 | 1.15636519 | 3.28E−09 |
| 8 | HML2_11q12.3b | 1.156119309 | 2.40E−05 |
| 5 | ERVLB4_5q13.3b | 1.156092227 | 4.19E−06 |
| 5 | HERVFRD_8q13.2 | 1.155993507 | 0.014451435 |
| 5 | HERVL_6p21.33b | 1.155695669 | 1.05E−07 |
| 5 | HERVL_12q21.2a | 1.155569721 | 7.80E−09 |
| 7 | HERVH_Xq13.1b | 1.155516788 | 2.92E−04 |
| 5 | MER4_14q32.2 | 1.155312748 | 0.019168766 |
| 2 | HML2_20q11.22 | 1.155247081 | 0.026533594 |
| 5 | HERVIP10F_11p14.3 | 1.155025604 | 0.003544784 |
| 6 | HERVH_3q24d | 1.15500208 | 0.025946342 |
| 7 | HERVH_7p21.1b | 1.154980939 | 0.04077846 |
| 7 | MER61_3p11.2 | 1.154967224 | 0.027975846 |
| 9 | HERVH_4q31.1b | 1.154867929 | 0.004268257 |
| 7 | HERVL_8q22.2 | 1.154635548 | 0.002288509 |
| 5 | HARLEQUIN_2q12.3 | 1.1545438 | 8.75E−06 |
| 5 | HML6_19p13.2d | 1.154171007 | 3.02E−07 |
| 5 | ERVLB4_2q32.3 | 1.153892795 | 0.005315286 |
| 5 | HERV4_19q13.43a | 1.153858444 | 4.16E−07 |
| 7 | HERVI_8p23.1 | 1.153713151 | 0.002074121 |
| 2 | HERVL_12p11.23 | 1.153548077 | 0.047495978 |
| 6 | HUERSP1_12q24.31b | 1.153380938 | 8.34E−04 |
| 6 | MER41_4q34.1a | 1.153333359 | 0.030958296 |
| 7 | HML1_19p12c | 1.153228004 | 0.024277639 |
| 6 | HERVH_7p22.1c | 1.153220903 | 0.00948956 |
| 5 | HERVH_12p13.2c | 1.153130763 | 0.001247105 |
| 5 | MER101_2p25.2 | 1.1530763 | 3.84E−08 |
| 6 | HERVH_12q21.31 | 1.152905574 | 0.049397707 |
| 5 | HML6_Xp11.21 | 1.152658873 | 1.04E−08 |
| 5 | HERVL_10q26.3b | 1.152582647 | 1.73E−08 |
| 9 | MER4_5p12c | 1.152525939 | 0.002413483 |
| 9 | HERVL74_19p13.2b | 1.152447595 | 0.04328197 |
| 8 | HERV9_7p14.2 | 1.152099726 | 1.47E−06 |
| 5 | HERVH48_19p12b | 1.151409081 | 2.25E−06 |
| 9 | MER4_7p14.3b | 1.151375749 | 0.024687878 |
| 8 | MER101_4p16.1 | 1.150960068 | 2.14E−05 |
| 5 | HERV9_18q11.2 | 1.150951425 | 0.007437502 |
| 5 | HARLEQUIN_1p36.33 | 1.150667317 | 7.23E−10 |
| 6 | HERVL_14q24.2c | 1.150599221 | 0.032342888 |
| 5 | MER61_18q21.2b | 1.150479473 | 2.66E−09 |
| 5 | MER61_4q31.21b | 1.150435394 | 7.16E−07 |
| 5 | ERVLB4_6p23 | 1.150229153 | 0.002011681 |
| 5 | HERVH_19q13.12b | 1.150166897 | 0.003209365 |
| 6 | HUERSP2_21q22.3 | 1.150076323 | 0.001758094 |
| 5 | HERVW_1p12a | 1.1499771 | 1.53E−06 |
| 8 | HERVEA_12p13.31 | 1.149937051 | 4.21E−04 |
| 9 | ERV316A3_6p22.1o | 1.149787578 | 0.027380295 |
| 5 | HUERSP1_4q21.22 | 1.149569704 | 2.86E−04 |
| 5 | HARLEQUIN_17q12 | 1.149154144 | 1.18E−05 |
| 5 | ERVLE_13q14.13 | 1.149118597 | 1.08E−08 |
| 8 | MER34B_4q31.3 | 1.148835971 | 4.15E−05 |
| 2 | HARLEQUIN_17q25.3b | 1.148552259 | 0.01280887 |
| 5 | HARLEQUIN_6p25.2 | 1.148469763 | 2.81E−04 |
| 5 | ERV316A3_7q21.3b | 1.148451636 | 1.57E−06 |
| 5 | HERV4_19q13.41 | 1.148387704 | 2.94E−07 |
| 4 | HERVS71_4p16.1b | 1.148333707 | 0.013562544 |
| 7 | HML5_2q31.1b | 1.148280977 | 0.0361787 |
| 4 | MER41_Xq11.1c | 1.148197683 | 0.018646355 |
| 2 | HERVL_5q33.1a | 1.148078676 | 0.001737683 |
| 2 | ERVLE_1q24.2 | 1.148046851 | 0.045284046 |
| 5 | HERVS71_8p23.1b | 1.147140675 | 0.026128503 |
| 5 | HERVP71A_4q31.21 | 1.146989671 | 7.06E−10 |
| 5 | HML5_16p11.2 | 1.146987314 | 3.67E−07 |
| 7 | ERV316A3_7q21.3b | 1.146790231 | 0.012749012 |
| 5 | PRIMA41_3q21.3b | 1.146675831 | 0.003113097 |
| 2 | HML5_15q21.2 | 1.146401106 | 0.039483566 |
| 5 | ERVLB4_12p13.2b | 1.146293368 | 2.11E−06 |
| 8 | ERV316A3_11q13.1a | 1.146192106 | 6.26E−08 |
| 5 | HERVE_11q13.4d | 1.146085461 | 9.28E−06 |
| 8 | HERVH_11q21a | 1.145774697 | 0.001543392 |
| 3 | HML5_12p13.31 | 1.144905875 | 0.013832324 |
| 8 | HERVW_2q13 | 1.14478495 | 0.002270856 |
| 5 | HERVS71_19q13.12a | 1.144760984 | 3.36E−07 |
| 4 | HML3_3q22.1 | 1.144716802 | 0.001608254 |
| 6 | ERVLB4_12p13.31e | 1.144548338 | 0.035359342 |
| 5 | HERVH_4p16.1a | 1.144478319 | 1.31E−05 |
| 5 | HERV9_7p14.1 | 1.144245227 | 0.006149449 |
| 5 | HUERSP2_19q13.2 | 1.144110653 | 7.43E−09 |
| 5 | HARLEQUIN_1q44 | 1.143996376 | 1.04E−05 |
| 7 | HERVI_9q34.13 | 1.143628791 | 0.01767765 |
| 7 | HERVL74_19p13.2b | 1.143528453 | 0.045576174 |
| 5 | ERVLE_19q13.31d | 1.143507581 | 1.01E−05 |
| 4 | HERVH48_14q24.2 | 1.143026056 | 0.002573115 |
| 5 | HML6_19q13.41e | 1.142705897 | 1.90E−08 |
| 5 | HML3_5p15.33d | 1.142613163 | 1.03E−06 |
| 8 | MER34B_10q25.3b | 1.142570743 | 0.040446642 |
| 9 | HERV3_19p13.3 | 1.142500246 | 3.73E−04 |
| 5 | HERVH_Xq13.1a | 1.142440788 | 2.74E−09 |
| 5 | HERVH_10q26.12 | 1.142097771 | 3.92E−04 |
| 5 | HERVH_17q25.1a | 1.141783494 | 1.89E−05 |
| 5 | ERV316A3_5q12.1b | 1.141776358 | 1.77E−05 |
| 5 | HERVH_4q35.2c | 1.141732557 | 2.51E−04 |
| 2 | HML5_14q31.1 | 1.141223946 | 0.012278006 |
| 5 | ERVLE_1q23.3a | 1.140984398 | 1.14E−06 |
| 8 | MER4B_12p13.31c | 1.140838235 | 6.99E−06 |
| 5 | HARLEQUIN_5p12a | 1.140810607 | 0.013160752 |
| 5 | HML2_1q22 | 1.140669794 | 1.94E−08 |
| 5 | ERVLB4_8p11.22a | 1.140614548 | 0.004837241 |
| 5 | HERVH_8p23.1c | 1.140565954 | 0.035015662 |
| 9 | HERVH_7q11.21 | 1.140441211 | 5.06E−09 |
| 5 | HERVH_19p12a | 1.140369961 | 0.008114349 |
| 5 | ERV316A3_6p22.1k | 1.140156267 | 1.67E−05 |
| 2 | HERVL_1q42.13 | 1.140121055 | 4.00E−04 |
| 5 | ERVLE_14q22.1 | 1.140083757 | 4.60E−06 |
| 5 | HML3_3p22.2 | 1.139896758 | 1.86E−06 |
| 6 | HERVH_2p14b | 1.139675305 | 3.98E−05 |
| 9 | MER4_7p14.3a | 1.139195098 | 0.03579599 |
| 6 | ERVLB4_1p34.2b | 1.139164513 | 9.09E−04 |
| 7 | PABLB_2q31.1 | 1.139047509 | 0.004740179 |
| 7 | HERV3_7q34a | 1.139039017 | 0.027623945 |
| 5 | ERV316A3_7p13a | 1.13836773 | 2.57E−04 |
| 5 | ERVLE_4q25h | 1.138280802 | 0.023306068 |
| 5 | HERVE_8p23.1h | 1.138055073 | 0.018519382 |
| 5 | HERVEA_1q31.1 | 1.138005545 | 0.016947142 |
| 5 | HML2_4p16.1a | 1.137895396 | 1.04E−05 |
| 5 | ERVLB4_3q22.1a | 1.137755272 | 3.86E−04 |
| 5 | MER61_8q23.1b | 1.136991618 | 2.46E−04 |
| 8 | ERVLB4_16p13.3a | 1.136791365 | 3.17E−06 |
| 7 | MER61_2q31.1b | 1.136772157 | 0.008115523 |
| 5 | HML3_3q27.2a | 1.136769387 | 2.19E−04 |
| 5 | LTR25_19q13.11 | 1.136568017 | 2.61E−08 |
| 5 | HERVK11_6q25.3 | 1.13656027 | 0.032261625 |
| 5 | HML6_19q13.41c | 1.136533894 | 2.82E−06 |
| 6 | HERVW_2q24.3 | 1.136405332 | 0.011830743 |
| 5 | HARLEQUIN_6q21 | 1.136296828 | 1.36E−04 |
| 5 | MER101_3p12.3 | 1.13591719 | 1.86E−06 |
| 5 | ERVL_16p12.2 | 1.135718731 | 3.25E−04 |
| 6 | ERVLE_2q31.1b | 1.135712043 | 0.019116475 |
| 6 | HERVEA_21q22.11 | 1.135663156 | 3.13E−08 |
| 5 | ERV316A3_2q37.1a | 1.135650194 | 3.11E−06 |
| 5 | ERV316A3_11q13.1b | 1.134685241 | 2.04E−09 |
| 5 | HERVH_15q26.3a | 1.134270267 | 1.58E−04 |
| 5 | HERVE_11q13.4e | 1.133929307 | 2.71E−05 |
| 5 | HARLEQUIN_7p15.3 | 1.133581621 | 0.001504245 |
| 5 | HML3_5p15.33c | 1.133574377 | 3.01E−06 |
| 5 | HERVIP10FH_11p15.5 | 1.13352076 | 1.73E−07 |
| 5 | ERV316A3_7q36.1a | 1.133506163 | 0.019170719 |
| 5 | HERVH48_1q44c | 1.13339763 | 2.35E−06 |
| 8 | ERV316A3_10q22.1a | 1.132879885 | 0.001535174 |
| 6 | ERVLB4_8p21.1b | 1.132831828 | 0.019466123 |
| 5 | HERVL_12p13.2a | 1.132815984 | 6.20E−06 |
| 5 | HERVH48_1q44b | 1.132116131 | 6.10E−05 |
| 2 | HERVH_17q25.3 | 1.131995626 | 0.019243235 |
| 5 | ERV316A3_11p15.2a | 1.131829903 | 0.039583213 |
| 5 | HERV4_13q32.3 | 1.131697764 | 1.68E−07 |
| 4 | MER4_5p12d | 1.131599309 | 0.004126345 |
| 5 | MER4_Xp22.2d | 1.1314399 | 1.59E−05 |
| 5 | ERV316A3_12q24.13 | 1.131048159 | 1.86E−07 |
| 9 | ERV316A3_6p22.1n | 1.130841038 | 0.007219536 |
| 4 | HML1_6q14.1 | 1.130537095 | 0.004839564 |
| 5 | ERV316A3_1q24.2a | 1.130496867 | 3.44E−04 |
| 4 | HML3_5p15.33d | 1.130469567 | 0.001843172 |
| 5 | HERVH_12p13.31d | 1.130326242 | 0.004652375 |
| 5 | HML6_Xp11.22 | 1.130166925 | 1.09E−06 |
| 5 | MER101_1p22.2a | 1.129882374 | 1.98E−07 |
| 9 | MER41_5p12 | 1.12959974 | 0.017445107 |
| 5 | LTR25_11p14.1 | 1.129281066 | 2.08E−05 |
| 7 | HERVE_7q21.3 | 1.129077323 | 1.71E−04 |
| 5 | ERV316A3_21q22.3e | 1.129013281 | 9.31E−05 |
| 5 | HERVH_9q21.33c | 1.128965927 | 1.51E−06 |
| 5 | HML3_16p13.13 | 1.128928417 | 1.07E−04 |
| 8 | MER4B_8q21.11 | 1.128830418 | 0.006038111 |
| 5 | HERVL_6q14.1d | 1.128748042 | 4.38E−04 |
| 2 | HERV3_11q13.3 | 1.128738584 | 0.006220723 |
| 5 | ERV316A3_3q13.12c | 1.128487506 | 4.05E−04 |
| 5 | MER41_8p11.22 | 1.128336934 | 4.03E−05 |
| 5 | ERVLE_13q12.11 | 1.128292231 | 0.001248901 |
| 5 | MER61_19p12c | 1.128192116 | 4.65E−04 |
| 4 | HERVFH21_1p34.3 | 1.127948407 | 0.00731108 |
| 5 | HERVH_10q24.31a | 1.12792756 | 2.61E−10 |
| 5 | HERVH_5q23.2g | 1.127873106 | 0.017162941 |
| 5 | HERVI_8p23.1 | 1.127849329 | 0.021090605 |
| 5 | ERVL_11p14.3 | 1.127767684 | 1.18E−08 |
| 6 | ERVLE_10p15.1d | 1.127168428 | 8.33E−05 |
| 5 | HERVEA_5q22.2 | 1.126795134 | 6.62E−05 |
| 5 | HUERSP3_2p25.2 | 1.126673909 | 2.19E−05 |
| 8 | ERV316A3_22q13.1 | 1.12659528 | 3.74E−05 |
| 5 | HML5_5p13.2 | 1.126585643 | 1.07E−04 |
| 5 | MER4_17q21.2d | 1.126527406 | 0.00412725 |
| 5 | MER4B_12q21.2 | 1.126106819 | 6.56E−07 |
| 5 | MER61_5p13.2 | 1.126096691 | 3.21E−06 |
| 5 | ERV316A3_2q35f | 1.125566784 | 2.74E−04 |
| 5 | ERVLE_2q24.3b | 1.124973969 | 0.017898054 |
| 5 | LTR19_19p13.2b | 1.124874888 | 1.32E−05 |
| 5 | HERV9_10p13 | 1.124824905 | 5.84E−10 |
| 5 | HERVL_12p13.2b | 1.124766682 | 0.006295864 |
| 5 | HML3_12q22 | 1.124708297 | 0.011543637 |
| 5 | HERVH_7q11.23a | 1.124630391 | 0.001772113 |
| 8 | MER41_1q44b | 1.124089083 | 5.09E−05 |
| 5 | HERVL_6q22.31c | 1.124022701 | 0.017158288 |
| 5 | HERVL40_1q41 | 1.123745362 | 0.003936834 |
| 5 | ERV316A3_6p22.1o | 1.123671277 | 1.77E−06 |
| 8 | ERVLB4_17q21.32 | 1.123539064 | 4.40E−04 |
| 6 | HERVL_5p13.3b | 1.123281608 | 7.64E−04 |
| 5 | ERVLB4_4p15.2a | 1.12316191 | 0.001194249 |
| 4 | HARLEQUIN_4q32.1 | 1.122935596 | 0.004941439 |
| 5 | ERV316A3_Xq28i | 1.122646539 | 2.05E−04 |
| 5 | HUERSP3_19q11 | 1.122415563 | 8.62E−05 |
| 5 | HML2_1q23.3 | 1.122135759 | 0.006089384 |
| 5 | HERVK11D_2q11.2 | 1.122024982 | 0.005750877 |
| 5 | HERV9_1q25.1 | 1.121793769 | 0.017001374 |
| 5 | MER101_12p13.31b | 1.121388051 | 4.82E−09 |
| 5 | MER4B_10q22.3 | 1.121254456 | 1.59E−04 |
| 5 | MER61_3p11.2 | 1.121101982 | 0.002936032 |
| 7 | HUERSP3_2q32.1 | 1.120901965 | 0.025705738 |
| 6 | HERVL74_2q11.2 | 1.120815612 | 0.001866797 |
| 5 | MER101_1p31.1f | 1.120557798 | 1.19E−04 |
| 8 | ERVLB4_8p21.3a | 1.120340311 | 3.97E−05 |
| 5 | ERVLB4_Xp11.21a | 1.119738833 | 0.008108331 |
| 8 | MER4_14q32.33a | 1.119720426 | 2.74E−04 |
| 5 | HML1_19p13.2 | 1.119538194 | 1.13E−04 |
| 5 | MER4_21q22.3a | 1.119478956 | 2.79E−04 |
| 8 | HML5_1q22 | 1.119324942 | 0.004434979 |
| 5 | ERVLE_15q26.3b | 1.119274862 | 6.68E−06 |
| 2 | HERVH_7p14.3d | 1.119087887 | 0.007483832 |
| 5 | HML5_2q31.1b | 1.118676773 | 6.88E−04 |
| 5 | HML3_19p12c | 1.118647873 | 0.035220503 |
| 8 | HML3_16p13.3 | 1.11830819 | 2.75E−05 |
| 5 | HML2_14q11.2 | 1.118237136 | 1.64E−05 |
| 5 | HERVP71A_6p22.1 | 1.118166333 | 9.06E−05 |
| 6 | MER41_8p11.22 | 1.117985947 | 0.047283958 |
| 9 | HERV3_7q11.21 | 1.117566944 | 1.12E−05 |
| 5 | HUERSP3B_Xq13.2b | 1.11677725 | 0.001725731 |
| 6 | HML5_2q14.3 | 1.116579053 | 4.66E−04 |
| 6 | MER41_12q13.11 | 1.116537071 | 0.0236639 |
| 4 | HML3_5p15.33c | 1.116163617 | 0.00515581 |
| 5 | HERVIP10FH_6q22.1 | 1.116086201 | 0.014226569 |
| 5 | MER101_6q27b | 1.116071224 | 0.014486966 |
| 5 | ERVLB4_8p12b | 1.115846267 | 0.005830633 |
| 8 | ERVLE_4q21.3 | 1.115487327 | 0.003520919 |
| 5 | HERVH_11p15.2 | 1.114783984 | 2.30E−04 |
| 8 | ERV316A3_11q13.1b | 1.114632359 | 5.37E−05 |
| 5 | HERVW_5p13.2a | 1.114575635 | 3.86E−04 |
| 5 | ERV316A3_11q13.1a | 1.114382167 | 1.20E−04 |
| 5 | HERVL18_6q22.31b | 1.114263251 | 0.043053096 |
| 5 | HERV4_4q22.1 | 1.113604342 | 8.50E−05 |
| 5 | ERV316A3_21q21.2g | 1.112974113 | 0.04246592 |
| 5 | HML5_Xq11.1 | 1.11183284 | 0.029720358 |
| 5 | ERVLE_6q21c | 1.111759759 | 0.005719401 |
| 5 | HML3_Xp11.21 | 1.111091324 | 0.036340773 |
| 5 | ERVL_Xq21.1a | 1.110194096 | 0.017948802 |
| 6 | ERV316A3_2p25.1b | 1.109931743 | 0.011009187 |
| 5 | HML6_19q13.43a | 1.109391963 | 4.36E−07 |
| 6 | ERVLB4_8p11.22a | 1.109378192 | 0.01712993 |
| 5 | HARLEQUIN_19p12b | 1.109062453 | 0.007971227 |
| 5 | MER61_1q23.1c | 1.10900854 | 8.20E−04 |
| 5 | HERVEA_19p13.11 | 1.108860953 | 0.001394563 |
| 5 | MER4B_Xq26.2b | 1.108621769 | 0.015918907 |
| 5 | HERVH_Xq13.1b | 1.107801994 | 5.05E−04 |
| 5 | HML1_6q14.1 | 1.107738375 | 0.0385476 |
| 5 | ERVLE_18q11.2c | 1.107641225 | 0.001964009 |
| 5 | HERVL40_1q44 | 1.107075664 | 0.008673666 |
| 5 | HERVP71A_12p13.31b | 1.106678934 | 0.042943489 |
| 5 | HERVP71A_2q32.2 | 1.105868332 | 4.61E−05 |
| 5 | HERV9_6p21.2 | 1.105845003 | 0.002879965 |
| 5 | HERVE_13q14.13b | 1.105662147 | 8.92E−07 |
| 5 | HERVH_6q22.1c | 1.105017132 | 0.001555788 |
| 5 | HERVW_1p22.2a | 1.10467562 | 0.009127625 |
| 5 | ERVLE_4q13.3g | 1.104292942 | 0.002701281 |
| 5 | ERV316A3_6p21.33c | 1.104257422 | 1.80E−04 |
| 5 | HML5_4q24 | 1.103994769 | 0.024640975 |
| 6 | MER41_14q32.32 | 1.103763698 | 0.002412307 |
| 5 | ERV316A3_2q22.3a | 1.103513785 | 2.79E−04 |
| 5 | HML3_19p13.2d | 1.103405268 | 0.004137909 |
| 5 | HERVH_14q22.1b | 1.103058052 | 0.005184023 |
| 5 | MER4_Xq24d | 1.102634538 | 0.001438653 |
| 5 | PABLB_2q31.1 | 1.102393872 | 0.044516254 |
| 5 | HML3_19p13.2e | 1.101427662 | 0.006231365 |
| 5 | ERVLB4_11p15.5d | 1.100885017 | 0.002189033 |
| 5 | MER101_2q35a | 1.100000357 | 0.023959959 |
| 5 | HERVS71_19q13.12b | 1.099893723 | 7.59E−04 |
| 5 | MER41_5p12 | 1.098964902 | 0.049676646 |
| 5 | LTR25_15q21.1a | 1.098914338 | 7.20E−04 |
| 5 | HUERSP3_2q32.1 | 1.098779652 | 0.018885504 |
| 8 | HERVL_11q12.1d | 1.098048973 | 0.013495803 |
| 6 | MER4_4p12a | 1.098033358 | 0.045509273 |
| 5 | MER4_3q29b | 1.097952163 | 0.043859906 |
| 6 | ERVLB4_7q36.3a | 1.097828432 | 0.045312195 |
| 5 | HML2_3q12.3 | 1.097266789 | 0.012917865 |
| 5 | MER4_1p31.3 | 1.097247091 | 6.03E−04 |
| 5 | HML3_17p13.1 | 1.096909035 | 0.024869558 |
| 5 | ERV316A3_18q11.2c | 1.096428235 | 0.028837065 |
| 8 | HML3_4q31.3 | 1.095276453 | 0.021176123 |
| 5 | HERVH_15q26.3b | 1.095172881 | 3.12E−08 |
| 5 | ERVLB4_19q13.12 | 1.093100327 | 0.026987341 |
| 5 | HERVIP10F_19p13.11 | 1.09281623 | 0.004905456 |
| 5 | ERV316A3_1p21.3a | 1.092419287 | 0.033187554 |
| 5 | MER4_Xq21.1a | 1.092293811 | 0.023153893 |
| 5 | HML2_1q21.3 | 1.091879493 | 0.004506743 |
| 5 | HERV3_7q33 | 1.091823491 | 2.39E−04 |
| 5 | HERVW_15q21.3 | 1.091570154 | 0.003973564 |
| 6 | ERVLE_3p12.2g | 1.091453103 | 0.012598502 |
| 5 | HARLEQUIN_7q33a | 1.091246789 | 4.56E−04 |
| 5 | ERV316A3_2q36.2 | 1.089768491 | 0.02921715 |
| 5 | HARLEQUIN_17q21.31 | 1.089525248 | 0.001894666 |
| 5 | HML3_19p12b | 1.089302683 | 0.035352512 |
| 5 | HERVIP10F_2q23.3 | 1.08878984 | 0.01606802 |
| 5 | PRIMA4_18q23b | 1.088192938 | 0.024428143 |
| 5 | MER61_2q31.1b | 1.086873406 | 0.005126952 |
| 5 | MER41_9q31.1a | 1.082297743 | 0.037754731 |
| 6 | ERVLE_3p12.2h | 1.080877155 | 0.038372754 |
| 5 | HERVE_7q21.3 | 1.079840117 | 0.015150701 |
| 5 | HML5_6p12.1 | 1.075744599 | 0.010038065 |
| 5 | PRIMA4_12p11.21b | 1.075555821 | 0.012797845 |
| 6 | HML5_14q31.1 | 0.947889277 | 0.031638856 |
| 8 | HERV3_7q11.21 | 0.944451017 | 0.024715214 |
| 6 | HERVH_15q26.3b | 0.942308928 | 0.028432859 |
| 6 | HERVL40_1q41 | 0.92379356 | 0.032201658 |
| 6 | ERVLB4_19q13.42a | 0.923561458 | 0.002129133 |
| 3 | HERVIP10F_2q23.3 | 0.923413073 | 0.039395198 |
| 6 | HERVL40_2q32.2 | 0.922697569 | 0.012764389 |
| 6 | HERVIP10FH_6q22.1 | 0.922443906 | 0.034741218 |
| 6 | HERVL_12p13.2a | 0.921437097 | 0.017083158 |
| 8 | HERVH_10q24.31a | 0.921135614 | 0.019911521 |
| 6 | MER101_12p13.31b | 0.920987919 | 0.01406931 |
| 6 | HERV3_7q11.21 | 0.91990694 | 1.11E−06 |
| 6 | HML5_6p12.1 | 0.91768731 | 5.47E−04 |
| 6 | HML3_16p13.3 | 0.917074151 | 0.016749275 |
| 3 | HERV9_10p13 | 0.916673617 | 0.00579939 |
| 6 | HERVH_6q22.1c | 0.916583803 | 0.019059294 |
| 6 | LTR57_14q22.1 | 0.914785651 | 0.004338645 |
| 6 | ERVLE_14q22.1 | 0.914392555 | 0.015480299 |
| 6 | MER4_1p31.3 | 0.914365115 | 0.002172432 |
| 3 | PRIMA4_12p11.21b | 0.91319071 | 0.012281354 |
| 8 | HML5_12q23.1 | 0.913181629 | 0.030884022 |
| 6 | MER41_5p12 | 0.912976815 | 8.28E−04 |
| 3 | HML3_4q31.3 | 0.912926289 | 0.023497517 |
| 3 | HUERSP3_2q32.1 | 0.911078302 | 0.002806512 |
| 6 | HERVS71_7p14.3 | 0.910809218 | 9.65E−04 |
| 6 | ERV316A3_7q21.3b | 0.910526257 | 0.005228604 |
| 6 | HML3_2q11.2 | 0.910195053 | 0.004752215 |
| 6 | HERVL18_6q22.31b | 0.90956761 | 3.17E−05 |
| 6 | HERVP71A_1q44b | 0.90831882 | 0.013272801 |
| 6 | HERVW_2p16.2 | 0.906876538 | 6.85E−05 |
| 3 | LTR23_7q11.23a | 0.906133358 | 0.016543454 |
| 3 | HERVH_9q21.33c | 0.905859962 | 0.017008082 |
| 6 | HERVH_12q13.2b | 0.904733044 | 0.002403946 |
| 6 | MER4_Xq24d | 0.90431512 | 0.00568157 |
| 3 | ERV316A3_1q21.2 | 0.903941603 | 0.015203053 |
| 3 | ERVLE_4q13.3g | 0.903551486 | 0.04410884 |
| 6 | ERV316A3_18q11.2c | 0.902880898 | 0.004957987 |
| 6 | HERVL_10q26.3b | 0.902840316 | 0.038385781 |
| 5 | MER61_4q26a | 0.901772063 | 0.012245925 |
| 6 | ERVLE_11q13.4g | 0.901346393 | 0.01689755 |
| 3 | MER4_7p14.3a | 0.900859061 | 0.029745513 |
| 3 | HML3_12q22 | 0.900676305 | 0.020636674 |
| 6 | MER41_1q44b | 0.900550916 | 0.005600062 |
| 6 | MER4_6q14.2 | 0.900348651 | 0.001808105 |
| 3 | HERVL18_2q32.1 | 0.89976074 | 0.008894322 |
| 3 | MER4B_10q22.3 | 0.899489776 | 0.02345121 |
| 6 | HML2_1q21.3 | 0.899412167 | 1.90E−04 |
| 3 | HERV9_13q34 | 0.899211 | 0.040361596 |
| 6 | MER61_19p12c | 0.89921094 | 0.005006109 |
| 5 | HERV4_11q21 | 0.898533918 | 2.48E−04 |
| 6 | LTR19_19p13.2b | 0.898083731 | 5.26E−05 |
| 3 | HERV9_1p31.1 | 0.897987788 | 0.023776758 |
| 3 | ERVLE_12p13.2c | 0.8979715 | 0.021109182 |
| 6 | HML2_3q21.2 | 0.897967871 | 0.027558795 |
| 6 | ERVLE_1p36.11b | 0.897092396 | 0.00433833 |
| 3 | HML5_2q31.1b | 0.896633188 | 7.39E−04 |
| 6 | MER4_7p14.3b | 0.896541662 | 0.004574746 |
| 6 | LTR25_12p11.22b | 0.896373575 | 0.016587481 |
| 6 | MER4_Xp22.2d | 0.89620764 | 2.32E−04 |
| 4 | ERVLE_8q11.23b | 0.895758827 | 5.43E−04 |
| 3 | HERVP71A_3p26.1 | 0.895655254 | 0.006419344 |
| 6 | HARLEQUIN_5q14.2 | 0.895253963 | 5.48E−04 |
| 8 | HERV9_10p13 | 0.895111052 | 0.00702832 |
| 8 | HML6_19q13.41c | 0.895023663 | 0.027644828 |
| 8 | ERVL_5q11.2 | 0.894919973 | 0.00570563 |
| 6 | HERVW_7q21.2 | 0.894823215 | 0.036085018 |
| 3 | HML5_14q31.1 | 0.894619538 | 0.023263173 |
| 6 | MER4_11q14.1 | 0.894406581 | 0.03767438 |
| 6 | HML5_Xq11.1 | 0.894333014 | 0.001969854 |
| 6 | HERVI_16p13.13 | 0.893857588 | 0.013523913 |
| 6 | HERVL_12p13.1c | 0.893733719 | 1.16E−04 |
| 3 | PRIMA4_10p12.1 | 0.893646512 | 0.02240745 |
| 3 | ERVLE_4q21.3 | 0.892768991 | 0.0042401 |
| 3 | HERVL40_1q44 | 0.892342097 | 0.023797132 |
| 3 | HUERSP3B_Xq13.2b | 0.892320529 | 0.017808976 |
| 6 | HERVW_5p13.2a | 0.892096254 | 2.81E−04 |
| 6 | HERVL_12q21.2a | 0.891865964 | 5.68E−04 |
| 6 | ERVLE_4q21.3 | 0.8917989 | 2.90E−04 |
| 6 | HML6_19q13.41c | 0.891149762 | 0.002236517 |
| 3 | HML2_1q22 | 0.891110972 | 0.025138873 |
| 6 | HML3_19p12b | 0.891001096 | 0.001404771 |
| 3 | MER101_4q23 | 0.889591622 | 0.002830846 |
| 3 | HERVE_2p14a | 0.889554495 | 0.004488181 |
| 6 | HERVH_7p14.3e | 0.889512893 | 0.015649909 |
| 8 | HML6_19q13.43a | 0.889443277 | 3.36E−06 |
| 6 | HERVH_4q35.2c | 0.889231862 | 0.006621672 |
| 6 | HML6_Xp11.21 | 0.889124272 | 2.70E−05 |
| 3 | HML5_2q14.3 | 0.889065946 | 0.006271802 |
| 8 | HERVH_7q11.21 | 0.888868944 | 5.82E−15 |
| 8 | PABLB_2q31.1 | 0.888433281 | 0.004393077 |
| 8 | MER4B_12q21.2 | 0.888379877 | 3.25E−04 |
| 6 | ERV316A3_4q31.1b | 0.888136999 | 3.20E−08 |
| 6 | LTR25_4q34.1a | 0.888100303 | 0.017445107 |
| 8 | HML3_17p13.1 | 0.887889213 | 0.008414709 |
| 8 | MER4B_Xq26.2a | 0.887825979 | 0.045816892 |
| 8 | HML6_Xp11.21 | 0.88775541 | 0.040892905 |
| 6 | MER101_19q13.2c | 0.887027059 | 0.025549684 |
| 6 | ERVLB4_19q13.12 | 0.886955406 | 0.001791255 |
| 6 | HERVL40_1q44 | 0.886913627 | 0.030102907 |
| 8 | HML1_6q22.32 | 0.88677343 | 0.034464423 |
| 8 | ERVLB4_7q36.3a | 0.886490568 | 0.015312757 |
| 4 | HERV3_3q21.3 | 0.886323044 | 0.044561853 |
| 6 | HERVL_9p21.3a | 0.885776279 | 0.01830221 |
| 8 | HML3_1p32.3 | 0.885509976 | 0.018307881 |
| 6 | HML3_1p32.3 | 0.885418369 | 0.012851916 |
| 3 | HERVL_4q24d | 0.885268239 | 2.20E−04 |
| 8 | LTR25_4q34.1a | 0.88509028 | 0.015543995 |
| 8 | HERV4_19q13.43a | 0.885069849 | 0.017981793 |
| 6 | LTR25_3p22.1 | 0.88406861 | 0.014429434 |
| 6 | MER4_5p12d | 0.884042349 | 3.03E−05 |
| 6 | HERV3_14q32.33 | 0.883884327 | 3.14E−06 |
| 8 | HERVE_13q14.13b | 0.883766675 | 1.52E−07 |
| 4 | LTR57_5q31.1 | 0.883764592 | 0.001347939 |
| 6 | MER4_1q21.2 | 0.883346193 | 9.31E−05 |
| 6 | LTR25_7q36.1 | 0.883249285 | 0.017003753 |
| 3 | HERV9_7p14.1 | 0.882728737 | 0.015137141 |
| 6 | HERVW_1p22.2a | 0.882520206 | 5.34E−04 |
| 6 | HERV4_19q13.41 | 0.882243185 | 0.005021985 |
| 8 | HERVL_12q21.2a | 0.881981392 | 0.014068859 |
| 6 | HERVH_10q24.31a | 0.881637134 | 8.89E−08 |
| 8 | ERVLE_6q21c | 0.88158102 | 0.005267561 |
| 4 | MER41_15q22.31b | 0.881224719 | 0.023890055 |
| 3 | LTR57_14q22.1 | 0.881177719 | 0.00715722 |
| 6 | HML5_15q21.2 | 0.881175741 | 9.46E−04 |
| 4 | HERV3_11p15.5 | 0.881046787 | 0.002203524 |
| 4 | HML3_1p21.3 | 0.880455747 | 0.037502793 |
| 3 | ERV316A3_7p13a | 0.880326615 | 0.010038065 |
| 6 | MER41_4p15.2 | 0.880313382 | 6.27E−06 |
| 6 | HML6_19q13.41f | 0.880169186 | 5.17E−04 |
| 6 | HERVH_3p22.3b | 0.880161076 | 0.0416414 |
| 6 | MER61_3p11.2 | 0.880071796 | 4.18E−06 |
| 3 | HML5_5p13.2 | 0.880025361 | 2.12E−04 |
| 3 | HERVK11_2q14.2 | 0.879711131 | 1.89E−05 |
| 8 | HERV3_7q34a | 0.879524026 | 5.23E−04 |
| 8 | ERVLE_4q24g | 0.879373941 | 0.002312247 |
| 6 | MER4_7p14.3a | 0.879145485 | 2.05E−05 |
| 6 | HERVIP10F_19p13.2b | 0.878621958 | 0.004695025 |
| 6 | HERVFRD_2p11.2 | 0.87819173 | 1.61E−04 |
| 6 | ERV316A3_8q24.21c | 0.878105265 | 7.33E−06 |
| 3 | ERVLB4_14q32.11b | 0.878067484 | 0.038681982 |
| 6 | ERV316A3_11q13.1b | 0.878013871 | 1.55E−07 |
| 8 | MER61_10q21.3 | 0.877916202 | 0.011525887 |
| 3 | MER41_5q15a | 0.877606579 | 0.009024771 |
| 8 | HML6_19p13.2d | 0.87738071 | 0.002676459 |
| 6 | ERV316A3_2q33.1g | 0.877283385 | 8.81E−07 |
| 6 | HML2_4p16.1b | 0.877133802 | 0.00841075 |
| 3 | HML3_7q36.1c | 0.877099536 | 0.004488181 |
| 3 | HERVL_9p21.3a | 0.876323086 | 0.002502964 |
| 6 | HERV3_11q13.3 | 0.87623567 | 1.00E−06 |
| 9 | HUERSP1_19q13.2a | 0.875804344 | 0.010111273 |
| 4 | HERVIP10F_12p13.2 | 0.875629686 | 0.004325499 |
| 3 | ERV316A3_9q21.33 | 0.875295163 | 0.010997713 |
| 3 | MER41_7q33 | 0.875163284 | 6.54E−04 |
| 6 | HARLEQUIN_2q12.3 | 0.874773127 | 0.003519939 |
| 6 | HERVEA_19p13.2 | 0.874361572 | 1.87E−08 |
| 8 | MER4B_Xq26.2b | 0.874322403 | 0.003806394 |
| 6 | ERVLE_15q26.3b | 0.874178824 | 6.17E−06 |
| 3 | HERVK11_2p14 | 0.873943231 | 3.06E−04 |
| 3 | HERVL_5p15.2a | 0.873748462 | 0.002557493 |
| 4 | ERV316A3_22q13.33a | 0.873600247 | 0.005580534 |
| 6 | HML5_5p13.2 | 0.873345509 | 2.29E−05 |
| 6 | HERVP71A_12p13.31b | 0.87321412 | 6.59E−06 |
| 6 | HERV4_13q32.3 | 0.872970472 | 4.93E−07 |
| 3 | PRIMA41_19p12d | 0.872547813 | 0.017156228 |
| 1 | HERVH_7q11.21 | 0.872332695 | 4.76E−05 |
| 6 | HARLEQUIN_17q12 | 0.872100623 | 9.86E−04 |
| 8 | HERVL_1q42.13 | 0.871553546 | 8.20E−07 |
| 3 | MER4_6p22.3d | 0.871138184 | 0.001213444 |
| 3 | ERVLE_1q24.2 | 0.870836901 | 4.31E−05 |
| 6 | HERVIP10F_19q13.41a | 0.870697496 | 4.40E−05 |
| 8 | HERVL40_9q34.3 | 0.870692547 | 4.11E−06 |
| 6 | HERVH_11p15.2 | 0.870539781 | 1.63E−05 |
| 8 | HERVW_5p13.2a | 0.870420254 | 0.036878041 |
| 8 | HERVP71A_4q31.21 | 0.870344618 | 2.89E−05 |
| 8 | ERV316A3_3q13.12c | 0.87024738 | 0.006563078 |
| 6 | ERVLE_9q33.2b | 0.869630924 | 1.17E−06 |
| 8 | ERVLE_13q12.13b | 0.869394326 | 0.002982281 |
| 3 | HERVK11_8p23.1c | 0.869199766 | 0.041942226 |
| 6 | ERVLE_14q12b | 0.869105341 | 1.87E−11 |
| 3 | HERVH_19q13.12a | 0.868688444 | 0.003204558 |
| 6 | HERVK11_1p13.3 | 0.868507396 | 9.98E−04 |
| 4 | ERVL_22q12.3b | 0.868344832 | 1.31E−04 |
| 6 | HML3_3p22.2 | 0.868179655 | 1.66E−05 |
| 8 | ERVLE_10p15.1d | 0.868137211 | 2.07E−04 |
| 8 | HERV4_19q13.43b | 0.867957074 | 0.016832868 |
| 6 | HERVE_11q13.4e | 0.867849803 | 7.32E−05 |
| 6 | HERVP71A_2q32.2 | 0.867807247 | 2.20E−08 |
| 6 | PRIMA41_19p12d | 0.867616623 | 0.016283043 |
| 8 | ERVL_1p34.2 | 0.867601279 | 0.022060836 |
| 8 | HML3_19p12b | 0.867134315 | 6.90E−05 |
| 6 | HERVE_11q13.4d | 0.866998555 | 2.45E−04 |
| 8 | HERVIP10F_1q44 | 0.866996356 | 0.005206028 |
| 6 | HERVW_1p12a | 0.866651423 | 1.69E−04 |
| 8 | HERVH48_1q44c | 0.866576994 | 1.65E−05 |
| 8 | HUERSP1_19q13.2a | 0.866569105 | 1.61E−04 |
| 5 | HERV9_3p24.3 | 0.86632779 | 5.34E−04 |
| 3 | HERV9_6p21.2 | 0.86630439 | 1.94E−06 |
| 8 | HERVL_12p11.23 | 0.866268644 | 0.008367019 |
| 3 | HERVW_7q21.2 | 0.866237919 | 2.92E−05 |
| 8 | MER4_5p12a | 0.866010556 | 2.90E−05 |
| 6 | HERVW_6q27b | 0.865958171 | 2.10E−05 |
| 6 | HUERSP3_19q11 | 0.865953618 | 2.52E−08 |
| 6 | HERVH_19p13.2a | 0.865760962 | 3.05E−06 |
| 3 | HML2_4p16.1b | 0.8656582 | 0.00407083 |
| 6 | ERV316A3_12q14.1e | 0.865586536 | 5.25E−07 |
| 3 | HERVL_12p11.23 | 0.865535214 | 0.004910963 |
| 1 | HML6_19q13.43a | 0.865361213 | 0.028643199 |
| 5 | MER4_1q44b | 0.865318929 | 0.005206058 |
| 8 | HERVFRD_6p24.2 | 0.864630772 | 4.37E−04 |
| 8 | MER4B_10q22.3 | 0.864622567 | 0.003418628 |
| 7 | HERVL40_13q32.1 | 0.864220031 | 0.022169246 |
| 6 | ERVLB4_3q22.1a | 0.864210028 | 6.39E−05 |
| 8 | HERVH_9q21.33c | 0.864159315 | 4.92E−05 |
| 2 | MER4B_Xq13.3 | 0.863768253 | 0.013955676 |
| 5 | MER4_15q21.2a | 0.863540544 | 4.03E−04 |
| 8 | HERVW_5p14.3 | 0.863526461 | 0.003918298 |
| 2 | ERV316A3_2p25.1a | 0.86348618 | 0.009427353 |
| 8 | ERVLE_2q24.3b | 0.863171589 | 0.001070099 |
| 8 | ERV316A3_6p22.1a | 0.863101172 | 0.001353178 |
| 8 | HERVH48_8q12.3 | 0.86306511 | 0.004904945 |
| 3 | ERV316A3_11p15.2a | 0.863053517 | 0.005099907 |
| 6 | HERVIP10F_19p13.11 | 0.862979074 | 1.94E−09 |
| 8 | HARLEQUIN_7p15.3 | 0.862884713 | 0.013131648 |
| 6 | HERV4_4q22.1 | 0.862748553 | 1.20E−06 |
| 4 | HERVH_2q33.2 | 0.862703355 | 0.002793377 |
| 3 | ERVL_16p12.2 | 0.862699802 | 5.52E−05 |
| 6 | HERVIP10F_15q14 | 0.862681076 | 1.14E−04 |
| 8 | HML5_1q42.12 | 0.862631919 | 0.006959266 |
| 6 | ERVLB4_Xp11.21a | 0.862512955 | 5.85E−05 |
| 3 | ERVLE_1p22.3c | 0.861506422 | 0.015209571 |
| 6 | HERVE_7q21.3 | 0.861364022 | 1.53E−10 |
| 4 | HERVK11_1q23.3a | 0.861283826 | 0.00750206 |
| 6 | MER41_22q12.3e | 0.860772431 | 4.39E−07 |
| 8 | HERVFH21_1p34.3 | 0.860736297 | 9.61E−05 |
| 7 | HML6_12q24.12a | 0.860670033 | 0.018227215 |
| 8 | ERVLB4_Xq11.1b | 0.860326698 | 0.035558595 |
| 6 | HERVL_Xq11.1a | 0.860101048 | 1.06E−09 |
| 7 | HUERSP1_12q24.31b | 0.859913309 | 0.004719323 |
| 8 | HML2_3p12.3 | 0.859889879 | 1.98E−04 |
| 6 | HERVH_9q21.33c | 0.859329817 | 1.51E−08 |
| 3 | MER61_3q28 | 0.858850618 | 0.001353178 |
| 8 | HML5_Xq11.1 | 0.858704904 | 0.034386387 |
| 8 | HERVK11D_2q11.2 | 0.858307239 | 0.011062378 |
| 8 | ERVLE_18q11.2c | 0.858080182 | 2.91E−05 |
| 6 | HERVH48_19p12b | 0.857860728 | 8.38E−05 |
| 6 | MER101_2p25.2 | 0.857799214 | 3.11E−07 |
| 6 | HERVH_8p23.1c | 0.857420457 | 0.041958941 |
| 6 | PABLA_18q23 | 0.857332078 | 8.13E−10 |
| 2 | HERVH_8q22.1a | 0.857266198 | 0.002234764 |
| 7 | HML3_5p15.33d | 0.857266183 | 2.16E−04 |
| 9 | HML6_6p22.2 | 0.857254526 | 5.68E−05 |
| 4 | HERVL66_4p16.3 | 0.857150568 | 0.012504378 |
| 7 | ERVLB4_17q25.1 | 0.856945168 | 0.018747512 |
| 6 | HML2_1q22 | 0.856895043 | 1.08E−08 |
| 3 | MER41_1q44b | 0.856468735 | 5.38E−08 |
| 7 | HERVS71_19p13.3 | 0.856338662 | 5.11E−05 |
| 6 | ERV316A3_6p22.1o | 0.856087084 | 8.12E−11 |
| 3 | HERVS71_4p16.1b | 0.855955309 | 1.41E−04 |
| 8 | HUERSP3_10q23.33b | 0.855880926 | 0.017984514 |
| 6 | HUERSP3_2q34 | 0.8558602 | 0.011557758 |
| 6 | HUERSP1_11p15.4b | 0.855777282 | 5.85E−07 |
| 3 | LTR25_7q36.1 | 0.855704914 | 6.64E−05 |
| 3 | MER4_2p21 | 0.854859097 | 4.41E−06 |
| 8 | HERVL_5q33.1a | 0.854519195 | 9.80E−04 |
| 3 | HERVH_1q42.2b | 0.854339689 | 0.001725078 |
| 2 | ERV316A3_2q36.2 | 0.854313634 | 4.79E−04 |
| 8 | ERV316A3_5q12.1b | 0.854195706 | 0.020607052 |
| 4 | MER101_2q33.1 | 0.854058274 | 0.010229785 |
| 8 | HUERSP3_2p13.3 | 0.853975633 | 2.66E−05 |
| 8 | HML2_4p16.1a | 0.853586475 | 9.06E−05 |
| 2 | MER4B_8q21.11 | 0.853051813 | 0.002264599 |
| 8 | MER41_5p12 | 0.853007047 | 9.20E−07 |
| 4 | MER41_8p11.22 | 0.852980694 | 1.14E−04 |
| 3 | HARLEQUIN_6p25.2 | 0.851563592 | 2.61E−04 |
| 8 | HERVE_20p11.21b | 0.851531904 | 0.019794595 |
| 8 | HERVH48_19p12b | 0.85125109 | 1.61E−04 |
| 8 | HERVI_8p23.1 | 0.851129894 | 7.76E−04 |
| 9 | HERVFH21_1p34.3 | 0.851052449 | 0.018832015 |
| 8 | HERV4_19p12 | 0.850867926 | 1.43E−04 |
| 8 | LTR25_2p13.3 | 0.850422176 | 2.44E−05 |
| 6 | HARLEQUIN_6p25.2 | 0.850069164 | 0.002689227 |
| 8 | HERVL_12q13.13 | 0.849850287 | 0.003912304 |
| 6 | HERVK11_8p23.1c | 0.849630226 | 0.007927801 |
| 1 | HML5_12q23.1 | 0.849607942 | 0.001839073 |
| 6 | ERV316A3_5q35.3a | 0.849445498 | 2.05E−04 |
| 8 | HUERSP3_10q23.33c | 0.849306723 | 0.002732805 |
| 8 | HERV9_7p14.1 | 0.849275968 | 3.93E−04 |
| 8 | HERVH_20p12.1c | 0.848549677 | 0.001302093 |
| 8 | PABLA_19q13.2 | 0.84847567 | 0.001557005 |
| 3 | MER41_1q44a | 0.848183653 | 1.22E−07 |
| 2 | HERV4_13q32.3 | 0.848086608 | 2.02E−05 |
| 6 | MER4_2p21 | 0.847460831 | 2.71E−07 |
| 8 | ERVLB4_5q13.3b | 0.847083401 | 2.42E−04 |
| 8 | HERV9_13q34 | 0.846544393 | 2.09E−06 |
| 6 | HERVFRD_9p21.3b | 0.846146428 | 9.75E−12 |
| 4 | PABLA_8p21.3a | 0.846011164 | 0.001070099 |
| 6 | MER4B_12p13.31c | 0.845642565 | 1.54E−06 |
| 6 | MER41_1q44a | 0.845617091 | 1.13E−09 |
| 2 | MER61_1q23.1c | 0.845473219 | 4.21E−04 |
| 6 | LTR25_7q11.21 | 0.845109549 | 2.83E−06 |
| 6 | HERVH_1q42.2b | 0.845040866 | 1.25E−05 |
| 8 | HERVFRD_8q13.2 | 0.844530236 | 0.03964373 |
| 7 | HML3_5p15.33c | 0.844301043 | 2.46E−06 |
| 8 | HERVL_6q22.31c | 0.843990992 | 0.003806545 |
| 8 | HERVL_6q14.1d | 0.843963717 | 1.55E−05 |
| 8 | MER41_12q13.11 | 0.843931571 | 1.92E−06 |
| 8 | HERVH_6q22.1c | 0.84361549 | 2.54E−09 |
| 4 | HERVK11_6q25.3 | 0.842786855 | 0.022604903 |
| 6 | HERV4_19q13.43b | 0.841620604 | 1.84E−05 |
| 6 | MER41_Xq11.1c | 0.841539309 | 7.71E−05 |
| 8 | HERVH_4q35.2c | 0.841122572 | 0.033429529 |
| 4 | HML2_1q23.3 | 0.840925512 | 4.18E−04 |
| 6 | HUERSP3_2p13.3 | 0.840627281 | 1.76E−07 |
| 3 | HERVI_8p23.1 | 0.840172046 | 1.96E−06 |
| 8 | ERV316A3_10q23.2a | 0.839928997 | 0.038755636 |
| 4 | MER34B_1q23.3a | 0.839781803 | 0.009568272 |
| 3 | MER4_11p15.4a | 0.839696796 | 4.49E−05 |
| 2 | ERV316A3_6p21.33c | 0.839675957 | 1.54E−06 |
| 7 | MER4_14q24.2c | 0.839643549 | 0.017983778 |
| 6 | HERVH_4p16.1a | 0.839241781 | 6.81E−07 |
| 8 | HUERSP3_10q23.33a | 0.839177154 | 0.001539962 |
| 4 | ERVL_1p36.22a | 0.838376056 | 1.43E−04 |
| 6 | ERV316A3_11q13.1a | 0.838254592 | 6.90E−12 |
| 6 | ERVLE_4q13.3g | 0.838251493 | 1.69E−11 |
| 8 | MER61_5p14.1 | 0.838146875 | 8.62E−05 |
| 6 | HERVL74_17q12 | 0.838031896 | 3.10E−06 |
| 8 | HUERSP3_2q32.1 | 0.837816969 | 4.59E−10 |
| 8 | HERVL18_14q11.2 | 0.837679734 | 7.44E−04 |
| 8 | HERVH_19p13.2a | 0.837411248 | 4.52E−04 |
| 4 | HML6_7p13 | 0.836910913 | 0.002965998 |
| 8 | HERVS71_11p15.4 | 0.836813113 | 0.005128286 |
| 2 | HERVW_3p21.31 | 0.836667278 | 0.014091503 |
| 7 | HERVH_1q25.3 | 0.836547329 | 0.045833768 |
| 7 | MER34B_10q25.3b | 0.836516162 | 0.019520017 |
| 6 | HERVS71_8p23.1b | 0.836475811 | 0.020014686 |
| 6 | HML2_4p16.1a | 0.835861819 | 3.54E−09 |
| 8 | HERVFH19_1p36.23b | 0.835585161 | 0.00458483 |
| 8 | HERV9_3p24.3 | 0.835508849 | 0.015316126 |
| 8 | HERVH_Xq22.2c | 0.834638664 | 2.21E−04 |
| 6 | HERVL_11q12.2 | 0.833908772 | 1.42E−06 |
| 6 | ERVLE_13q12.11 | 0.833896801 | 6.37E−08 |
| 6 | HERVW_15q21.3 | 0.833351726 | 1.02E−13 |
| 2 | MER34B_1q23.3a | 0.832608936 | 0.007062692 |
| 4 | HERVI_8p23.1 | 0.832517985 | 2.99E−04 |
| 6 | MER101_16p12.2a | 0.832483652 | 1.39E−06 |
| 8 | MER41_9p13.3a | 0.832435342 | 1.34E−06 |
| 6 | ERV316A3_2q37.1a | 0.832273901 | 7.06E−10 |
| 4 | ERV316A3_11p15.2a | 0.83136485 | 0.034022141 |
| 2 | LTR25_3p22.1 | 0.831229941 | 0.02055559 |
| 8 | HERVL_17q12b | 0.831194716 | 0.01163328 |
| 8 | HERVH_20q13.12a | 0.830893022 | 4.08E−05 |
| 3 | HERVH_5q13.3b | 0.830816646 | 0.004230127 |
| 8 | MER61_6q14.1b | 0.830757296 | 0.031624486 |
| 8 | HERVE_3q22.1b | 0.830655102 | 0.001231198 |
| 8 | HERVH_13q13.3b | 0.830462842 | 0.004819699 |
| 8 | HML2_7q34 | 0.829719996 | 6.68E−07 |
| 4 | ERV316A3_9q21.33 | 0.829674863 | 0.005792675 |
| 6 | PRIMA4_10p12.1 | 0.82965388 | 4.43E−12 |
| 9 | MER41_12q13.11 | 0.829588304 | 0.01429567 |
| 6 | LTR25_2p13.3 | 0.829307829 | 2.28E−09 |
| 2 | HERVH_3p21.31e | 0.829242102 | 0.001922737 |
| 6 | HARLEQUIN_6q21 | 0.829198103 | 6.54E−08 |
| 7 | MER101_Xq23b | 0.829014926 | 0.029320569 |
| 8 | HERVL18_6p22.1a | 0.828901549 | 5.93E−13 |
| 8 | HERV9_6q25.1 | 0.828668888 | 9.68E−04 |
| 8 | ERVLB4_1p34.2b | 0.828549345 | 9.61E−06 |
| 8 | HERVH_Xq22.2b | 0.828085605 | 4.37E−04 |
| 8 | ERV316A3_6p22.1m | 0.827914533 | 3.45E−04 |
| 9 | HARLEQUIN_1p36.33 | 0.827879728 | 0.025541366 |
| 6 | HML3_16p13.13 | 0.827842814 | 1.66E−10 |
| 8 | HERVE_9q34.3 | 0.827420679 | 0.00797564 |
| 9 | HERVH_2q24.1d | 0.827109651 | 0.013540359 |
| 6 | HML3_6q21a | 0.827034052 | 1.49E−07 |
| 4 | ERVLE_2q24.3b | 0.826377651 | 6.42E−05 |
| 8 | HERV3_14q32.33 | 0.826337554 | 5.39E−13 |
| 8 | ERVLE_13q12.11 | 0.826313529 | 1.45E−07 |
| 6 | HERVE_8p23.1h | 0.826002725 | 9.11E−05 |
| 4 | MER41_6q22.31b | 0.825969086 | 0.044516254 |
| 8 | HML2_3q13.2 | 0.825915655 | 0.020834071 |
| 6 | ERVLE_4p12e | 0.825722536 | 3.68E−09 |
| 7 | MER61_2q35 | 0.825686253 | 0.01004593 |
| 8 | MER61_18q12.3a | 0.825590248 | 7.93E−07 |
| 8 | HERV9_5q23.3 | 0.825352495 | 4.43E−04 |
| 6 | HARLEQUIN_17q21.31 | 0.825301522 | 2.73E−18 |
| 8 | HERVH_12q22 | 0.825142115 | 1.42E−05 |
| 6 | MER101_2q35a | 0.825140089 | 5.12E−10 |
| 8 | ERVLE_11q14.2a | 0.825136297 | 9.71E−13 |
| 8 | ERVLB4_1p22.3a | 0.825022569 | 0.031946137 |
| 8 | HERVH_Xq13.1b | 0.824267132 | 3.96E−13 |
| 6 | HUERSP3B_11p12 | 0.824199551 | 8.78E−13 |
| 8 | MER41_4p15.2 | 0.824153676 | 3.13E−08 |
| 6 | ERVLE_13q14.13 | 0.82399728 | 4.41E−13 |
| 8 | LTR25_Yq11.223d | 0.823724213 | 0.015231731 |
| 9 | ERV316A3_12q24.13 | 0.823673481 | 9.83E−05 |
| 6 | ERVLB4_11p15.5c | 0.823541176 | 4.64E−10 |
| 6 | HERVS71_19q13.12a | 0.823513331 | 1.86E−10 |
| 6 | ERVLE_19q13.31c | 0.823427474 | 1.19E−10 |
| 8 | MER101_19q13.2c | 0.82333437 | 0.001017108 |
| 8 | HERVH_11p15.4b | 0.822647485 | 0.001406278 |
| 7 | MER61_10q21.3 | 0.822398633 | 0.003912386 |
| 8 | HERVE_3p26.3 | 0.822313196 | 0.008131559 |
| 8 | HERVL66_19p12a | 0.822054379 | 2.50E−10 |
| 6 | HERVH_Xq13.1b | 0.821414128 | 1.24E−13 |
| 9 | ERVL_22q12.3b | 0.821365193 | 0.03864218 |
| 8 | HML2_3q21.2 | 0.821364868 | 4.73E−05 |
| 2 | HUERSP2_19q13.2 | 0.820894318 | 6.73E−09 |
| 6 | HERV4_19q13.43a | 0.820758543 | 2.64E−10 |
| 4 | HUERSP3_19q13.42 | 0.819725426 | 0.013578789 |
| 8 | ERV316A3_2p25.1b | 0.819644716 | 4.26E−05 |
| 6 | HERVH_Xq13.1a | 0.818998433 | 2.34E−15 |
| 8 | HERVL_21q22.2 | 0.81880118 | 2.91E−06 |
| 8 | MER4_3q13.11 | 0.818597969 | 0.001125338 |
| 7 | HERVH_6p21.2b | 0.818502212 | 6.27E−04 |
| 8 | HERVE_11q13.4c | 0.81829506 | 7.19E−06 |
| 6 | ERVLE_19q13.31d | 0.817914598 | 4.03E−14 |
| 4 | HERVH_19q13.32e | 0.817584218 | 4.52E−05 |
| 8 | HERVH_21q22.3 | 0.817213165 | 7.71E−08 |
| 6 | MER4_11p15.4a | 0.817141479 | 1.04E−08 |
| 4 | ERVLB4_8p11.22a | 0.816900856 | 2.31E−06 |
| 9 | HERVH_11q24.1c | 0.816730384 | 0.029400433 |
| 7 | HUERSP1_11p15.4a | 0.81662033 | 0.048838884 |
| 4 | ERVLB4_1p36.22 | 0.815771247 | 5.18E−07 |
| 8 | HERVW_4p16.3 | 0.815677793 | 6.26E−05 |
| 8 | HERVP71A_1q44b | 0.815399437 | 4.09E−05 |
| 8 | MER4_14q22.3a | 0.81525503 | 2.19E−05 |
| 6 | HERVL74_19p13.2b | 0.815069111 | 1.32E−14 |
| 9 | HARLEQUIN_4q32.1 | 0.815059547 | 0.003049421 |
| 8 | HERVL_12q13.11b | 0.814964575 | 1.45E−06 |
| 8 | MER4_10q23.31b | 0.814818529 | 0.024469382 |
| 8 | HERVW_2q24.3 | 0.814375249 | 7.11E−05 |
| 2 | HERVEA_12p13.31 | 0.813989194 | 0.001719065 |
| 8 | HML3_6q21a | 0.813937894 | 3.64E−04 |
| 8 | HERVFRD_18p11.23 | 0.813533967 | 6.01E−04 |
| 7 | ERV316A3_1p32.3a | 0.813210267 | 9.50E−04 |
| 8 | HERVEA_3p24.3b | 0.813075911 | 6.46E−04 |
| 9 | HERVE_2p14a | 0.813023109 | 0.009513255 |
| 8 | HML5_7p14.1 | 0.812953118 | 3.80E−07 |
| 2 | ERVLB4_17q21.32 | 0.812942066 | 1.20E−07 |
| 6 | HERVH48_21q22.3 | 0.812928432 | 3.57E−11 |
| 2 | ERVLB4_17q25.1 | 0.812763914 | 7.83E−06 |
| 8 | HERVEA_21q21.1 | 0.81183666 | 9.32E−10 |
| 8 | HERVH_11q13.4b | 0.811451265 | 0.006456417 |
| 8 | HUERSP3_19q11 | 0.811295325 | 1.95E−07 |
| 8 | HERVL_9q21.11a | 0.811160714 | 0.013389728 |
| 8 | HML3_5p12a | 0.811123468 | 2.15E−05 |
| 8 | HERVE_7p22.1c | 0.810329686 | 4.25E−04 |
| 2 | HERVH_3q13.2 | 0.809964452 | 0.045934255 |
| 8 | HERVL_1p13.3b | 0.809124537 | 2.16E−04 |
| 8 | HERVH_10q23.31b | 0.809113513 | 0.031899442 |
| 9 | HERVK11_2q14.2 | 0.808514007 | 0.00984378 |
| 6 | MER34B_9p21.3b | 0.808507519 | 9.08E−10 |
| 2 | HERVIP10F_19p13.2b | 0.808132809 | 3.19E−04 |
| 8 | HERVS71_19p12b | 0.808095536 | 0.004330407 |
| 4 | MER4_11q21d | 0.807578218 | 7.26E−05 |
| 2 | HERVH48_2q22.2 | 0.807437328 | 0.002947557 |
| 8 | HERVH_Xq13.1a | 0.807362094 | 1.68E−16 |
| 1 | HERVH_12q13.2b | 0.806868006 | 0.002186843 |
| 6 | HERVL_5q12.3 | 0.806099911 | 9.52E−13 |
| 8 | HERVH_12q15b | 0.80586165 | 0.046493742 |
| 6 | HML2_3q12.3 | 0.805469995 | 1.99E−13 |
| 7 | HUERSP3_19q13.42 | 0.805416362 | 0.012848791 |
| 8 | HERVS71_19q13.12b | 0.805313944 | 6.52E−17 |
| 8 | HML3_19p12c | 0.805154284 | 1.20E−07 |
| 8 | HERVFH21_Xq21.1 | 0.805112479 | 1.93E−04 |
| 1 | HERVS71_19p13.3 | 0.805043459 | 0.006669785 |
| 8 | HERVH_3q21.3d | 0.804783905 | 6.53E−05 |
| 8 | HML3_19p13.2d | 0.804765791 | 3.60E−14 |
| 8 | HML1_6q14.1 | 0.804459186 | 2.06E−06 |
| 2 | HUERSP3_19q13.42 | 0.803532688 | 0.031595453 |
| 8 | HERVW_2q32.3 | 0.803342729 | 3.63E−04 |
| 8 | HERVH_1q24.2 | 0.80325261 | 1.10E−06 |
| 1 | MER4_21q22.3a | 0.803148753 | 0.001048344 |
| 8 | HERVFH21_19p12 | 0.802599139 | 2.25E−09 |
| 8 | LTR25_Yq11.223h | 0.801958689 | 0.001052659 |
| 8 | HERVW_Xp11.21 | 0.801909849 | 1.97E−08 |
| 8 | MER41_6q21b | 0.801516827 | 0.002362957 |
| 8 | HERVL_4p13a | 0.800335591 | 3.48E−04 |
| 8 | ERVLB4_Xp11.23b | 0.800298643 | 0.014661293 |
| 8 | HERVH_8q22.1c | 0.800045906 | 1.21E−07 |
| 8 | ERV316A3_6q14.1a | 0.799634344 | 1.41E−04 |
| 6 | HML6_Xp11.22 | 0.799564989 | 3.30E−15 |
| 4 | MER4_10q23.31b | 0.799387658 | 0.010958291 |
| 8 | HERVH_5p14.3a | 0.799099744 | 1.05E−06 |
| 8 | ERVLB4_8p21.1b | 0.798889734 | 1.14E−06 |
| 4 | MER34B_19p13.11 | 0.798565122 | 4.93E−06 |
| 8 | ERVLB4_2q32.3 | 0.798451846 | 1.84E−06 |
| 2 | HUERSP3_9p23 | 0.798202909 | 0.005601299 |
| 8 | HERVH_2p16.1e | 0.797982957 | 7.12E−05 |
| 8 | HML3_Xq21.1a | 0.797364017 | 0.001490284 |
| 8 | HERVH_6p22.3a | 0.797282802 | 5.81E−11 |
| 8 | HERVS71_19q13.12a | 0.796819612 | 2.34E−14 |
| 8 | LTR19_Yq11.223a | 0.79658239 | 0.002405914 |
| 8 | HML3_7q11.21 | 0.796485193 | 1.58E−09 |
| 6 | MER4B_Xq26.2b | 0.79646139 | 1.59E−13 |
| 8 | HARLEQUIN_18p11.22a | 0.796324181 | 6.92E−05 |
| 6 | HARLEQUIN_1q42.13c | 0.796030262 | 9.60E−16 |
| 8 | ERVLE_11q13.1b | 0.795713088 | 5.37E−06 |
| 8 | MER41_12p13.2 | 0.794915302 | 1.14E−05 |
| 8 | ERVLB4_2q13a | 0.794621598 | 0.017984908 |
| 6 | HERVL_7p15.2a | 0.794621521 | 2.79E−11 |
| 7 | ERVLB4_2q13c | 0.794446838 | 5.49E−05 |
| 2 | PABLA_19q13.2 | 0.794319415 | 8.95E−07 |
| 8 | HML6_3p21.31b | 0.794239419 | 9.08E−05 |
| 8 | HML2_11q12.1 | 0.79400958 | 9.13E−09 |
| 8 | HERVE_17q11.2 | 0.793895834 | 5.48E−08 |
| 8 | LTR25_Yq11.223g | 0.793745226 | 3.03E−04 |
| 6 | ERV316A3_21q22.3e | 0.793715024 | 4.85E−15 |
| 6 | HERVEA_19p13.11 | 0.793438412 | 5.81E−17 |
| 4 | HERVH_12q21.31 | 0.793273264 | 0.02431533 |
| 8 | HERV3_19p12b | 0.79318943 | 1.81E−11 |
| 1 | HML6_14q24.2 | 0.793154205 | 0.012595552 |
| 8 | HML3_19p13.2e | 0.793087111 | 2.50E−16 |
| 2 | ERV316A3_21q21.2g | 0.792641524 | 1.81E−06 |
| 8 | ERVLE_8q22.3b | 0.792041904 | 4.39E−09 |
| 8 | HERV9_7q21.11 | 0.791158739 | 2.51E−04 |
| 8 | HML5_14q32.33b | 0.791046425 | 3.67E−04 |
| 9 | HERVH_11q13.4b | 0.790993829 | 0.021097877 |
| 8 | HERVH_7p22.1c | 0.790808276 | 8.78E−07 |
| 2 | HERVK11_1q23.3a | 0.790143141 | 7.25E−05 |
| 4 | MER4_14q32.2 | 0.789312138 | 7.99E−05 |
| 8 | MER61_3p11.2 | 0.789289092 | 1.65E−09 |
| 4 | MER4_11q21e | 0.788974837 | 1.27E−07 |
| 4 | ERVLB4_12p13.31e | 0.788718687 | 0.001015908 |
| 9 | HERVL_9q21.33b | 0.788624397 | 0.004803669 |
| 8 | HERVH_19q13.12b | 0.787566134 | 2.76E−05 |
| 2 | MER61_4p16.3 | 0.787407883 | 0.006021948 |
| 2 | MER34B_1q23.3b | 0.787192158 | 0.002704582 |
| 8 | HERVH_12q13.2b | 0.787184407 | 2.06E−19 |
| 8 | HERVS71_14q32.33 | 0.786420512 | 1.18E−06 |
| 8 | HERV3_11p15.5 | 0.786365335 | 2.10E−15 |
| 8 | HERVL_1p22.2d | 0.785398754 | 1.00E−13 |
| 8 | HERVL_12p13.31b | 0.785282633 | 8.30E−04 |
| 9 | HARLEQUIN_2q32.1 | 0.784966638 | 3.41E−04 |
| 8 | HERVP71A_12p13.31a | 0.784942242 | 5.01E−10 |
| 9 | MER4_14q22.3a | 0.784710144 | 0.003590796 |
| 8 | HERVH_2p24.3a | 0.784671472 | 6.95E−06 |
| 8 | HUERSP2_1q32.1 | 0.784460588 | 5.78E−12 |
| 8 | HARLEQUIN_19p12b | 0.784300902 | 2.98E−10 |
| 2 | HML6_3p21.31b | 0.784191098 | 0.002856142 |
| 8 | HERVE_4p16.1c | 0.783976563 | 3.96E−06 |
| 8 | ERV316A3_1q25.2b | 0.78385046 | 9.75E−05 |
| 8 | ERVLE_1p22.3c | 0.783821183 | 2.07E−07 |
| 8 | HERVH_10p15.1 | 0.783496697 | 3.35E−08 |
| 8 | PRIMA4_7q11.21b | 0.782176075 | 4.11E−05 |
| 2 | HERVH_6q12f | 0.781862216 | 0.001114351 |
| 8 | HERVH_4p16.1b | 0.781746198 | 4.30E−05 |
| 2 | HERVIP10FH_5q35.1 | 0.781188312 | 5.97E−05 |
| 8 | HERVL74_8q24.13 | 0.780812646 | 6.37E−05 |
| 8 | HERVH_6p21.2b | 0.780474765 | 4.42E−10 |
| 8 | HERVS71_11q13.4 | 0.780275918 | 1.30E−04 |
| 1 | HERVH_10q24.31a | 0.780243223 | 2.17E−06 |
| 9 | HERV4_4q12 | 0.780122215 | 1.07E−08 |
| 8 | HERVH_22q11.1 | 0.779736768 | 2.20E−06 |
| 8 | MER61_4q31.21a | 0.778977604 | 1.12E−06 |
| 8 | HERVH_19p13.12 | 0.778763112 | 2.08E−14 |
| 8 | MER4_4p12a | 0.778554317 | 3.89E−18 |
| 8 | HUERSP3_9p23 | 0.778412948 | 2.34E−06 |
| 2 | HERVL18_3p21.31b | 0.778098193 | 0.003158638 |
| 8 | HERVH_4q31.21c | 0.77704999 | 3.13E−09 |
| 9 | MER4B_Xq13.3 | 0.776922937 | 1.50E−04 |
| 8 | HERVIP10F_11p14.3 | 0.776733339 | 4.78E−08 |
| 9 | HERVH_1q42.13c | 0.776641958 | 0.012556009 |
| 7 | ERVLB4_2q32.3 | 0.776362728 | 9.10E−07 |
| 1 | ERVLE_4q24g | 0.775781303 | 0.011332293 |
| 8 | HML5_19p12a | 0.775591614 | 3.87E−08 |
| 8 | HERVH_12p13.2c | 0.775326239 | 1.91E−06 |
| 8 | HERVL66_4p16.3 | 0.775174539 | 1.53E−11 |
| 8 | ERVL_11p14.3 | 0.775152699 | 6.10E−19 |
| 9 | MER4B_8q21.11 | 0.774987986 | 2.12E−04 |
| 9 | HUERSP3B_1q21.3 | 0.774783819 | 0.023468727 |
| 6 | HERVE_Xp11.23 | 0.774517794 | 1.13E−17 |
| 8 | HML1_19p13.2 | 0.774286085 | 1.26E−17 |
| 8 | HML1_19p12c | 0.773492261 | 9.31E−18 |
| 6 | HARLEQUIN_1q32.1 | 0.773340066 | 4.08E−15 |
| 8 | MER61_19p12c | 0.773308478 | 4.61E−12 |
| 8 | MER61_4p16.3 | 0.772348316 | 6.04E−07 |
| 8 | HERVS71_4p16.1a | 0.772169155 | 1.88E−04 |
| 8 | HERVH_19p12a | 0.771832228 | 2.10E−10 |
| 2 | HML5_1q22 | 0.771639049 | 3.68E−09 |
| 6 | HERVH_5q13.3b | 0.770992734 | 2.14E−08 |
| 6 | HML6_19p13.2d | 0.77040337 | 6.48E−17 |
| 8 | HML6_19q13.41e | 0.770336429 | 1.69E−17 |
| 9 | ERVLB4_17q25.1 | 0.76982538 | 2.28E−06 |
| 8 | HERVH_10q23.31c | 0.769527473 | 7.70E−04 |
| 8 | HERVL74_2q11.2 | 0.769181623 | 1.23E−12 |
| 8 | HERVI_11p15.4 | 0.768839181 | 1.59E−11 |
| 8 | HERVH_4p16.3b | 0.76860031 | 4.99E−10 |
| 2 | HERVH_14q32.2c | 0.768030097 | 0.004159108 |
| 8 | HERVH_11q13.4a | 0.767725785 | 1.45E−07 |
| 2 | ERV316A3_2q35f | 0.767448163 | 3.32E−07 |
| 2 | HERVL18_3p21.31a | 0.767285469 | 0.001706269 |
| 8 | MER4_4q23b | 0.766974262 | 3.36E−24 |
| 6 | MER4B_Xq26.2a | 0.76628666 | 8.91E−17 |
| 1 | HERVH_6p21.2b | 0.766186104 | 0.028773881 |
| 9 | HERVS71_11q13.4 | 0.765899495 | 0.001288967 |
| 8 | HARLEQUIN_1q24.2 | 0.765876477 | 4.48E−10 |
| 7 | HARLEQUIN_Xq23b | 0.765698278 | 1.81E−04 |
| 2 | MER4_10q23.31b | 0.765458077 | 0.001055287 |
| 8 | ERVLE_10q25.3a | 0.764775643 | 1.50E−10 |
| 9 | HML3_3p22.2 | 0.764696789 | 1.61E−05 |
| 8 | MER41_12p12.1b | 0.764294392 | 2.23E−10 |
| 8 | HERVL_10q26.3b | 0.763136909 | 4.52E−16 |
| 9 | HERVL40_13q32.1 | 0.76305944 | 0.003658427 |
| 8 | ERVLB4_14q31.3b | 0.763027562 | 1.94E−11 |
| 1 | HERVIP10FH_14q23.1 | 0.762814265 | 0.004638216 |
| 2 | HML6_7p13 | 0.762581078 | 6.80E−09 |
| 8 | HERVH_4q22.1c | 0.761707856 | 2.97E−07 |
| 6 | HERVH_Xq26.2 | 0.759809617 | 4.77E−15 |
| 8 | HERVH_7p21.1b | 0.759482646 | 2.21E−11 |
| 9 | HERVW_2q24.3 | 0.75947733 | 9.92E−04 |
| 8 | MER4_19p12a | 0.759131504 | 1.93E−17 |
| 7 | HERVE_1p36.12 | 0.758777181 | 5.82E−09 |
| 9 | ERVLB4_10q26.2b | 0.758225613 | 0.01925328 |
| 9 | HERV3_4q13.3b | 0.757789408 | 0.003366197 |
| 4 | HERVH_22q11.1 | 0.757628308 | 1.87E−05 |
| 1 | PRIMA4_7q11.21b | 0.7570568 | 0.014385435 |
| 1 | HERVEA_21q21.1 | 0.755701475 | 0.001452503 |
| 8 | HARLEQUIN_1q32.1 | 0.755101941 | 5.48E−16 |
| 9 | HERVH_19q13.42b | 0.754169513 | 0.001358681 |
| 8 | HERVW_19p12a | 0.753907715 | 3.84E−14 |
| 8 | MER41_4q34.1a | 0.753847648 | 1.81E−08 |
| 9 | ERV316A3_6p22.1a | 0.753475921 | 2.00E−06 |
| 4 | HERVH_10q23.31b | 0.75330509 | 6.63E−04 |
| 9 | HERVH_Xq22.2b | 0.752793492 | 0.001043139 |
| 8 | ERV316A3_8q11.21a | 0.752786547 | 6.95E−06 |
| 6 | HERVH_19p13.2d | 0.752582387 | 1.46E−15 |
| 9 | HERVE_17q11.2 | 0.752364294 | 0.003093686 |
| 9 | HERVE_10p13c | 0.751867203 | 2.45E−04 |
| 8 | HERVH_4q13.2 | 0.751320414 | 2.15E−13 |
| 8 | HARLEQUIN_1q44 | 0.750676472 | 2.11E−17 |
| 8 | MER41_7q11.21i | 0.75012652 | 4.21E−07 |
| 1 | ERVLE_1p36.12 | 0.749452253 | 5.20E−04 |
| 1 | HML2_3q21.2 | 0.749028397 | 0.015177099 |
| 9 | HERVH_6q12f | 0.748975562 | 0.02432957 |
| 8 | HERVL_3p24.3d | 0.748966092 | 3.56E−05 |
| 9 | ERV316A3_7q36.1a | 0.748579483 | 0.005153082 |
| 1 | ERVLB4_1p34.2b | 0.747392942 | 0.001064843 |
| 1 | MER41_9p13.3a | 0.747387377 | 0.047859317 |
| 2 | HERVH_10q23.31b | 0.747211892 | 0.001518023 |
| 1 | HML2_3p12.3 | 0.746489974 | 0.019406416 |
| 1 | HERVH48_8q24.13 | 0.746399679 | 0.020027098 |
| 4 | ERV316A3_1p32.3a | 0.745873587 | 1.70E−07 |
| 9 | HERVH_19q13.32e | 0.745027151 | 1.71E−06 |
| 9 | LTR25_Xq22.2b | 0.744666656 | 0.043175769 |
| 2 | HML2_10p14 | 0.744055167 | 0.017873012 |
| 8 | HERVIP10FH_19p12c | 0.743805996 | 4.70E−20 |
| 2 | HML2_1q32.2 | 0.743606857 | 2.31E−06 |
| 8 | MER4B_14q32.33d | 0.743584098 | 3.14E−15 |
| 8 | ERVLE_14q11.2d | 0.741612381 | 2.64E−14 |
| 1 | HERVFH21_1p36.31 | 0.741100096 | 0.02160872 |
| 1 | ERVLB4_11p15.5d | 0.739044662 | 3.70E−06 |
| 1 | HML2_11q12.3b | 0.737976411 | 0.005314464 |
| 9 | HERVH_3p21.31c | 0.736396159 | 0.00775149 |
| 9 | HML5_6p22.2 | 0.736384031 | 2.32E−04 |
| 2 | HERVW_3p11.1 | 0.735961965 | 3.11E−06 |
| 8 | HERV3_19p12e | 0.735810268 | 3.97E−13 |
| 1 | HML2_4p16.1b | 0.735396549 | 3.90E−04 |
| 8 | ERVLE_19q13.2b | 0.735153785 | 1.43E−15 |
| 8 | HERVKC4_19q13.41 | 0.733629774 | 9.92E−16 |
| 9 | HERVL_Xq23b | 0.733215626 | 0.047938411 |
| 8 | LTR46_19q13.43 | 0.733075106 | 3.66E−11 |
| 9 | MER4B_Xq23a | 0.732932793 | 0.01167062 |
| 5 | HUERSP3_8q11.1 | 0.732442983 | 2.82E−05 |
| 9 | HERVW_10q24.1 | 0.732204098 | 0.002664165 |
| 1 | HERVH_1q25.3 | 0.731403195 | 0.014255421 |
| 9 | HERVI_11p15.4 | 0.731015435 | 0.00521961 |
| 9 | HERVL_1p12 | 0.73085075 | 2.20E−06 |
| 9 | MER4_10p12.33 | 0.730395069 | 0.042307734 |
| 9 | HERVH_19q13.31 | 0.729787643 | 0.009009206 |
| 9 | HERVS71_2q14.3b | 0.729772424 | 7.94E−04 |
| 9 | HARLEQUIN_18p11.22b | 0.729221768 | 5.68E−05 |
| 9 | HERVL74_8q24.13 | 0.728573902 | 0.004825899 |
| 6 | HERVFC1_7q11.21 | 0.728531711 | 2.64E−18 |
| 1 | HML2_11p15.4a | 0.72707318 | 3.35E−05 |
| 9 | HUERSP3_6p21.32 | 0.7266053 | 0.001213444 |
| 8 | HERVW_14q11.2 | 0.726528013 | 1.56E−10 |
| 9 | HERVH_6q12c | 0.726410781 | 0.040942748 |
| 9 | HERVH_Xq22.2c | 0.726219832 | 2.54E−04 |
| 8 | HERVW_19p12b | 0.725966327 | 9.83E−14 |
| 1 | HERVE_7p22.1c | 0.725252249 | 0.003015989 |
| 9 | PRIMA41_8q24.3 | 0.725170745 | 9.14E−04 |
| 9 | ERVLE_12p13.31d | 0.72516171 | 0.018718112 |
| 1 | HML3_2p24.1 | 0.725157496 | 0.007674088 |
| 8 | ERVLB4_4q12d | 0.725052892 | 3.54E−09 |
| 2 | HUERSP3_2q34 | 0.724988143 | 4.97E−06 |
| 9 | HERVH_17q25.1a | 0.724752096 | 4.62E−09 |
| 1 | HERVFH21_Xq21.1 | 0.722710555 | 0.01022303 |
| 1 | HERVFH21_1q23.1 | 0.721330328 | 0.001007415 |
| 9 | HERVE_2p14b | 0.720161335 | 1.46E−04 |
| 8 | ERVL_Xq21.1a | 0.71969923 | 7.49E−24 |
| 8 | HERVL_11q12.1c | 0.718994511 | 2.70E−22 |
| 8 | HERVS71_2q14.3b | 0.718992037 | 6.64E−12 |
| 9 | HERVL_12q12b | 0.718922419 | 9.17E−05 |
| 9 | HERVH_4q22.3a | 0.718552771 | 0.001604687 |
| 6 | HML3_3q22.1 | 0.718491805 | 8.90E−27 |
| 1 | HERVH_4p16.1d | 0.718091474 | 0.041558138 |
| 1 | HERVH_21q22.3 | 0.717927164 | 3.51E−04 |
| 8 | MER4_Xq21.1a | 0.71755941 | 8.35E−25 |
| 9 | HERVH_6q23.2a | 0.717180736 | 0.00509175 |
| 9 | HERVH_6q23.2b | 0.717138359 | 0.00112549 |
| 9 | ERVLB4_8p23.1f | 0.716762412 | 0.007806291 |
| 9 | HERVH48_11p15.4 | 0.715401048 | 0.00122795 |
| 1 | ERVLB4_9q34.11 | 0.714276201 | 0.039330274 |
| 9 | MER34B_19p13.11 | 0.714040289 | 7.87E−07 |
| 1 | HERVH_13q13.3b | 0.713197138 | 0.006759928 |
| 8 | HML6_19p12e | 0.712315828 | 2.23E−16 |
| 1 | ERV316A3_2p25.1b | 0.711640363 | 5.73E−05 |
| 9 | HERVH_4q21.21a | 0.711303818 | 0.010770014 |
| 1 | MER4_15q21.2a | 0.709286293 | 0.00263121 |
| 1 | HERVKC4_19q13.41 | 0.708293702 | 0.001131621 |
| 1 | HERVH_6q12f | 0.708290931 | 0.004957987 |
| 9 | LTR25_12p11.22b | 0.708168823 | 3.47E−09 |
| 9 | HERVW_1q32.1 | 0.707220024 | 0.021950841 |
| 9 | HERVL_18p11.21a | 0.706551899 | 0.010051423 |
| 1 | HERVH_7p22.1c | 0.705409686 | 9.36E−04 |
| 9 | MER61_4q31.21a | 0.703994544 | 1.58E−04 |
| 8 | HML2_10p14 | 0.703975279 | 1.79E−08 |
| 9 | HERVW_8q12.3b | 0.703947185 | 2.41E−04 |
| 8 | ERVLE_2q34e | 0.702828745 | 7.16E−20 |
| 1 | HML3_7q11.21 | 0.702824391 | 5.33E−06 |
| 9 | HERVS71_19p12b | 0.702553375 | 0.002617209 |
| 1 | ERVLB4_1p36.11 | 0.702042151 | 5.19E−05 |
| 1 | ERV316A3_3q13.12c | 0.701920801 | 6.84E−05 |
| 1 | HERVH_8q22.1c | 0.701303369 | 0.003330805 |
| 1 | HERVE_19q12 | 0.700481394 | 0.038708906 |
| 9 | ERV316A3_22q13.1 | 0.700025005 | 1.20E−11 |
| 1 | HERVE_20p11.21b | 0.699899053 | 3.65E−05 |
| 1 | HERVS71_19p12b | 0.699797635 | 0.024271441 |
| 9 | MER4_14q24.2c | 0.699788959 | 1.72E−06 |
| 1 | HERVIP10FH_Xp21.1 | 0.69907924 | 0.024812898 |
| 9 | HARLEQUIN_18p11.22a | 0.697998273 | 0.003734441 |
| 1 | HERVE_11q13.4c | 0.697912087 | 5.76E−05 |
| 1 | HERVH_20p13c | 0.697391014 | 0.001151954 |
| 1 | MER61_12q15a | 0.696602571 | 0.024247739 |
| 1 | HERVH_11p15.4b | 0.695708514 | 5.30E−04 |
| 9 | ERV316A3_13q13.3c | 0.693028499 | 1.41E−04 |
| 1 | HERVIP10FH_11p15.5 | 0.692220953 | 1.27E−07 |
| 9 | HERV9_4q13.3 | 0.69167697 | 0.005602705 |
| 9 | ERV316A3_8q11.21a | 0.689366266 | 0.001705828 |
| 9 | HERVH_14q11.2b | 0.687508472 | 0.035776527 |
| 1 | ERVLE_11q13.1b | 0.686749816 | 6.29E−04 |
| 9 | HERVH_1q32.1a | 0.686298511 | 2.18E−05 |
| 1 | HERVE_11p15.4 | 0.683895038 | 4.54E−06 |
| 8 | HARLEQUIN_2q32.1 | 0.683810276 | 6.49E−28 |
| 9 | HERVH_12p11.22a | 0.683403194 | 1.30E−10 |
| 8 | HML2_22q11.23 | 0.68237722 | 4.38E−19 |
| 1 | HERVL_12q14.2a | 0.679615553 | 0.00101743 |
| 9 | ERV316A3_6q22.31g | 0.67915615 | 1.34E−04 |
| 1 | HML3_19q13.31 | 0.678713572 | 6.49E−04 |
| 1 | ERVLE_Xq26.3h | 0.678651224 | 0.019395966 |
| 9 | HERVH_5p14.3a | 0.67856466 | 1.07E−04 |
| 8 | HERVL_21q21.1e | 0.67840634 | 3.39E−14 |
| 8 | MER41_7q34b | 0.677106425 | 8.12E−20 |
| 1 | ERVLB4_13q12.12a | 0.676899777 | 0.013908417 |
| 9 | ERVLB4_12p13.31e | 0.676755553 | 1.97E−06 |
| 9 | HERVH_14q24.2a | 0.676072955 | 2.86E−05 |
| 1 | HERVL_12q13.13 | 0.673359262 | 2.63E−06 |
| 9 | HERVL40_8q11.23b | 0.671665963 | 0.001177102 |
| 1 | HERV9_7q21.13 | 0.670868733 | 1.80E−04 |
| 1 | HERVL40_13q32.1 | 0.670431902 | 0.003133898 |
| 1 | HML5_19p12a | 0.670223813 | 1.30E−04 |
| 1 | MER4_14q32.2 | 0.669756442 | 6.46E−05 |
| 9 | ERVLE_11q23.3c | 0.668871138 | 1.40E−06 |
| 9 | HERVEA_1q31.1 | 0.667050863 | 1.23E−07 |
| 9 | HERVH_8q24.22a | 0.666066725 | 1.13E−04 |
| 9 | HERVH_12p13.31e | 0.66498698 | 2.54E−06 |
| 1 | ERVLB4_2q13c | 0.661502835 | 0.022804878 |
| 1 | HERVS71_11p15.4 | 0.660285009 | 1.61E−05 |
| 9 | MER61_2q35 | 0.658202794 | 2.27E−05 |
| 9 | LTR25_12p11.22a | 0.656628995 | 7.71E−11 |
| 9 | ERVLE_Xq26.3h | 0.654072376 | 3.40E−05 |
| 1 | HERVH_11q13.4a | 0.653519637 | 6.73E−06 |
| 1 | HML1_Xq26.3 | 0.652730473 | 0.004645562 |
| 9 | MER41_4q34.1a | 0.649667737 | 6.76E−05 |
| 9 | HERVL_11q12.1d | 0.648771185 | 2.45E−08 |
| 1 | MER101_1q42.13a | 0.648068001 | 4.67E−06 |
| 9 | MER34B_10q25.3b | 0.647579684 | 9.35E−08 |
| 1 | ERV316A3_1q25.2b | 0.647294346 | 0.001702311 |
| 9 | HERVP71A_12p13.33b | 0.645520596 | 4.10E−08 |
| 9 | HERVI_12p13.31 | 0.638635099 | 1.44E−06 |
| 1 | HERVW_19p12b | 0.638302845 | 2.16E−05 |
| 9 | HERVH_10q23.31c | 0.62560874 | 6.34E−06 |
| 8 | HERVH_4q22.3a | 0.625104635 | 1.27E−21 |
| 9 | HARLEQUIN_11q12.2b | 0.624048084 | 6.25E−11 |
| 9 | HERVH48_2q22.2 | 0.623579985 | 3.43E−08 |
| 1 | HERVW_8q12.3b | 0.622433904 | 1.61E−05 |
| 9 | HML1_Xq26.3 | 0.622235183 | 4.90E−06 |
| 9 | HERVH_10q23.31d | 0.621952891 | 1.10E−07 |
| 9 | HERVW_2q22.2 | 0.619269898 | 5.77E−06 |
| 9 | HERVL_21q21.1e | 0.618713573 | 9.09E−07 |
| 1 | HERVH_12q21.31 | 0.617878945 | 1.04E−04 |
| 9 | HML2_11q12.1 | 0.616669623 | 8.80E−11 |
| 9 | HUERSP3_9p23 | 0.613738928 | 4.58E−10 |
| 9 | HERVH_10q23.31b | 0.613084101 | 1.66E−06 |
| 9 | MER4_10q23.31b | 0.612716651 | 6.71E−08 |
| 9 | HML3_Xq21.1a | 0.610835594 | 1.07E−09 |
| 1 | ERVLB4_2q32.3 | 0.602190058 | 2.22E−06 |
| 1 | HUERSP3_9p23 | 0.600255322 | 2.35E−06 |
| 1 | HERVL_18p11.21a | 0.598739972 | 9.25E−07 |
| 9 | HERVH_4q31.21c | 0.59142126 | 1.24E−10 |
| 9 | HERVH_20p13c | 0.586651276 | 8.59E−13 |
| 9 | ERVLB4_2q32.3 | 0.584196735 | 3.91E−11 |
| 9 | HUERSP3_19q13.42 | 0.581266068 | 4.96E−11 |
| 9 | ERV316A3_3q27.3e | 0.580360302 | 3.71E−14 |
| 8 | HERVK11_4q13.2 | 0.580220432 | 1.77E−26 |
| 1 | HERVH_22q11.1 | 0.578610944 | 7.10E−08 |
| 9 | MER4_4q25a | 0.578555499 | 3.17E−11 |
| 9 | MER61_10q21.3 | 0.569593707 | 5.54E−15 |
| 9 | HERVL18_11q12.2 | 0.55887821 | 6.41E−15 |
| 9 | MER4_4q25b | 0.556351561 | 2.31E−15 |
| 1 | ERVLE_8q12.3f | 0.540427299 | 4.27E−10 |
| 9 | HERVL_3q25.1 | 0.512882911 | 2.79E−16 |
| 9 | HERVW_3p11.1 | 0.502116724 | 1.62E−13 |

TABLE 2

| | | Start position | End position |
|---|---|---|---|
| Herv_id | Chromosome | on chromosome | on chromosome |
| HARLEQUIN_1p36.33 | chr1 | 1412252 | 1418852 |
| HERVFH21_1p36.31 | chr1 | 6560297 | 6570283 |
| HERVFH19_1p36.23b | chr1 | 9088458 | 9096029 |
| ERVLB4_1p36.22 | chr1 | 9297468 | 9302644 |
| ERVL_1p36.22a | chr1 | 9302227 | 9304444 |
| HERVE_1p36.12 | chr1 | 20154110 | 20160610 |
| ERVLE_1p36.12 | chr1 | 21453383 | 21453988 |
| ERVLB4_1p36.11 | chr1 | 24888544 | 24893132 |
| ERVLE_1p36.11b | chr1 | 26811581 | 26816449 |
| ERVLE_1p35.1c | chr1 | 32928071 | 32931352 |
| HERVFH21_1p34.3 | chr1 | 36329861 | 36337342 |
| ERVL_1p34.2 | chr1 | 41174282 | 41175563 |
| ERVLB4_1p34.2b | chr1 | 42904887 | 42912582 |
| ERV316A3_1p32.3a | chr1 | 52152823 | 52159748 |
| HML3_1p32.3 | chr1 | 54630287 | 54636120 |
| MER4_1p31.3 | chr1 | 66912485 | 66919266 |
| HERVP71A_1p31.1a | chr1 | 71502068 | 71510867 |
| HERV9_1p31.1 | chr1 | 74740033 | 74745644 |
| MER101_1p31.1f | chr1 | 84186630 | 84190130 |
| ERVLE_1p22.3c | chr1 | 85806974 | 85813032 |
| ERVLB4_1p22.3a | chr1 | 85913180 | 85915546 |
| HERVW_1p22.2a | chr1 | 88924529 | 88931881 |
| MER101_1p22.2a | chr1 | 89048082 | 89049754 |
| HERVL_1p22.2d | chr1 | 89437349 | 89443684 |
| HERVH_1p22.1 | chr1 | 93830337 | 93837088 |
| HML3_1p21.3 | chr1 | 94444820 | 94450789 |
| ERV316A3_1p21.3a | chr1 | 97679722 | 97680969 |
| HERVL_1p13.3b | chr1 | 108018546 | 108023153 |
| ERVLE_1p13.3b | chr1 | 108065479 | 108067847 |
| HERVK11_1p13.3 | chr1 | 109702615 | 109709643 |
| HERVW_1p12a | chr1 | 119168299 | 119171363 |
| HERVL_1p12 | chr1 | 120001503 | 120003474 |
| PRIMA4_1q21.2c | chr1 | 148367053 | 148369350 |
| MER4_1q21.2 | chr1 | 148409788 | 148410690 |
| ERV316A3_1q21.2 | chr1 | 150165565 | 150170972 |
| HML2_1q21.3 | chr1 | 150632886 | 150634662 |
| HUERSP3B_1q21.3 | chr1 | 153603714 | 153606012 |
| HML2_1q22 | chr1 | 155626666 | 155635845 |
| HML5_1q22 | chr1 | 156184304 | 156189713 |
| HERVFH21_1q23.1 | chr1 | 157273298 | 157279886 |
| HML5_1q23.1 | chr1 | 158947555 | 158952739 |
| HERVIP10FH_1q23.1 | chr1 | 159022297 | 159025513 |
| MER61_1q23.1c | chr1 | 159056048 | 159059052 |
| HERVK11_1q23.3a | chr1 | 160686556 | 160690784 |
| HML2_1q23.3 | chr1 | 160690785 | 160700016 |
| HERVK11_1q23.3b | chr1 | 160700017 | 160703548 |
| ERVLE_1q23.3a | chr1 | 161295147 | 161297592 |
| MER34B_1q23.3a | chr1 | 161514473 | 161517154 |
| MER34B_1q23.3b | chr1 | 161596130 | 161598811 |
| ERV316A3_1q24.2a | chr1 | 167581655 | 167584948 |
| ERVLE_1q24.2 | chr1 | 167732967 | 167735856 |
| HARLEQUIN_1q24.2 | chr1 | 167863091 | 167869903 |
| HERVH_1q24.2 | chr1 | 169683070 | 169688724 |
| HERV9_1q25.1 | chr1 | 173367863 | 173382451 |
| ERV316A3_1q25.2b | chr1 | 177455894 | 177461782 |
| HERVH_1q25.3 | chr1 | 183613210 | 183619373 |
| HERVEA_1q31.1 | chr1 | 186914372 | 186923133 |
| HUERSP2_1q32.1 | chr1 | 198827111 | 198831700 |
| HML1_1q32.1 | chr1 | 205857564 | 205862572 |
| HERVW_1q32.1 | chr1 | 205866332 | 205871494 |
| HERVH_1q32.1a | chr1 | 205871746 | 205875092 |
| HML3_1q32.1b | chr1 | 205890691 | 205896160 |
| HARLEQUIN_1q32.1 | chr1 | 206052519 | 206058912 |
| ERVLE_1q32.2d | chr1 | 207512891 | 207513462 |
| HML2_1q32.2 | chr1 | 207635112 | 207639291 |
| MER41_1q41a | chr1 | 215583131 | 215585190 |
| HERVL40_1q41 | chr1 | 219218772 | 219223420 |
| HML5_1q42.12 | chr1 | 225221660 | 225229271 |
| HARLEQUIN_1q42.13c | chr1 | 227677124 | 227686201 |
| HERVL_1q42.13 | chr1 | 228250669 | 228253090 |
| MER101_1q42.13a | chr1 | 228648618 | 228650136 |
| HERVH_1q42.13c | chr1 | 228947424 | 228951887 |
| HERVH_1q42.2b | chr1 | 231829672 | 231835648 |
| MER41_1q44a | chr1 | 244815534 | 244818297 |
| MER41_1q44b | chr1 | 244818602 | 244821738 |
| HUERSP3_1q44 | chr1 | 246529011 | 246538336 |
| HERVH48_1q44b | chr1 | 246634716 | 246640099 |
| HERVIP10F_1q44 | chr1 | 246747512 | 246752385 |
| HERVL40_1q44 | chr1 | 247144115 | 247148678 |
| HERVH48_1q44c | chr1 | 247286141 | 247290862 |
| HERVP71A_1q44b | chr1 | 247354022 | 247360572 |
| HARLEQUIN_1q44 | chr1 | 247898331 | 247905895 |
| MER4_1q44b | chr1 | 248773924 | 248775499 |
| HUERSP3_2p25.2 | chr2 | 6838364 | 6846887 |
| MER101_2p25.2 | chr2 | 6869773 | 6876489 |
| ERV316A3_2p25.1a | chr2 | 8296771 | 8302648 |
| ERV316A3_2p25.1b | chr2 | 11395773 | 11400825 |
| HERVH_2p24.3a | chr2 | 12569680 | 12575439 |
| HML3_2p24.1 | chr2 | 19922747 | 19928511 |
| MER4_2p21 | chr2 | 42773889 | 42783034 |
| HERVW_2p16.2 | chr2 | 53756643 | 53761425 |
| HERVH_2p16.1e | chr2 | 60417435 | 60422485 |
| HERVK11_2p14 | chr2 | 63988445 | 63997140 |
| HERVH_2p14b | chr2 | 64252414 | 64257646 |
| HERVE_2p14a | chr2 | 64887412 | 64891023 |
| HERVE_2p14b | chr2 | 64897102 | 64899872 |
| HERVH_2p13.3a | chr2 | 69443011 | 69449341 |
| LTR25_2p13.3 | chr2 | 69790238 | 69794493 |
| HUERSP3_2p13.3 | chr2 | 69794782 | 69798802 |
| HERVFRD_2p11.2 | chr2 | 88679963 | 88686725 |
| HML3_2q11.2 | chr2 | 99270681 | 99278573 |
| HERVL74_2q11.2 | chr2 | 100276117 | 100280003 |
| HERVK11D_2q11.2 | chr2 | 101237832 | 101245098 |
| HARLEQUIN_2q12.3 | chr2 | 108523896 | 108531463 |
| ERVLB4_2q13a | chr2 | 109458529 | 109463559 |
| ERVLB4_2q13c | chr2 | 110379003 | 110381368 |
| HERVW_2q13 | chr2 | 112039346 | 112044989 |
| HERVK11_2q14.2 | chr2 | 119373755 | 119376815 |
| HERVS71_2q14.3b | chr2 | 123686372 | 123691521 |
| HML5_2q14.3 | chr2 | 127369875 | 127375395 |
| HML6_2q14.3 | chr2 | 127615267 | 127618672 |
| HERVW_2q22.2 | chr2 | 142899925 | 142907899 |
| HERVH48_2q22.2 | chr2 | 142930835 | 142937291 |
| ERV316A3_2q22.3a | chr2 | 143751094 | 143753311 |
| HERVIP10F_2q23.3 | chr2 | 152701339 | 152709137 |
| HERVH_2q24.1d | chr2 | 157340836 | 157343474 |
| HERVH_2q24.1e | chr2 | 157343501 | 157345447 |
| HUERSP2_2q24.2 | chr2 | 162324003 | 162329299 |
| HERVW_2q24.3 | chr2 | 164657911 | 164659611 |
| ERVLE_2q24.3b | chr2 | 165779881 | 165783164 |
| HML5_2q31.1b | chr2 | 172513063 | 172519738 |
| PABLB_2q31.1 | chr2 | 172520800 | 172524411 |
| MER61_2q31.1b | chr2 | 172537081 | 172542095 |
| ERVLE_2q31.1b | chr2 | 173119652 | 173124224 |
| HUERSP3_2q32.1 | chr2 | 185963079 | 185965965 |
| HERVL18_2q32.1 | chr2 | 186431386 | 186438154 |
| HARLEQUIN_2q32.1 | chr2 | 187510490 | 187515862 |
| HERVP71A_2q32.2 | chr2 | 190213418 | 190219869 |
| HERVL40_2q32.2 | chr2 | 190587122 | 190590915 |
| ERVLB4_2q32.3 | chr2 | 192042435 | 192046614 |
| HERVW_2q32.3 | chr2 | 195334677 | 195340075 |
| LTR25_2q32.3a | chr2 | 196124161 | 196131057 |
| MER101_2q33.1 | chr2 | 196851115 | 196854775 |
| ERV316A3_2q33.1g | chr2 | 199772560 | 199775568 |
| HERVH_2q33.2 | chr2 | 203961942 | 203967634 |
| HUERSP3_2q34 | chr2 | 208389905 | 208398026 |
| ERVLE_2q34e | chr2 | 213044484 | 213048020 |
| ERV316A3_2q35f | chr2 | 216034255 | 216036830 |
| MER101_2q35a | chr2 | 216213296 | 216216431 |
| MER61_2q35 | chr2 | 218793927 | 218798599 |
| ERV316A3_2q36.2 | chr2 | 224827741 | 224830374 |
| ERV316A3_2q37.1a | chr2 | 230403818 | 230406889 |
| HERVE_2q37.1 | chr2 | 231400712 | 231408843 |
| HERVL40_2q37.3 | chr2 | 241017184 | 241020543 |
| HERVE_3p26.3 | chr3 | 846924 | 853933 |
| HERVP71A_3p26.1 | chr3 | 5140823 | 5149820 |
| HERV9_3p24.3 | chr3 | 17774262 | 17782406 |
| HERVEA_3p24.3b | chr3 | 19484214 | 19490699 |
| HERVL_3p24.3d | chr3 | 22023929 | 22030341 |
| HERVH_3p22.3b | chr3 | 32460939 | 32466915 |
| HML3_3p22.2 | chr3 | 38420760 | 38427256 |
| LTR25_3p22.1 | chr3 | 42918608 | 42924975 |
| HERVH_3p21.31c | chr3 | 46246275 | 46252065 |
| HERVH_3p21.31e | chr3 | 46387685 | 46393402 |
| HERVL18_3p21.31a | chr3 | 46417556 | 46419247 |
| HERVL18_3p21.31b | chr3 | 46419310 | 46423900 |
| HML6_3p21.31b | chr3 | 46426543 | 46433630 |
| HERVW_3p21.31 | chr3 | 48330526 | 48333802 |
| HML2_3p12.3 | chr3 | 75551314 | 75559999 |
| MER101_3p12.3 | chr3 | 75709487 | 75712912 |
| ERVLE_3p12.2g | chr3 | 81728971 | 81730583 |
| ERVLE_3p12.2h | chr3 | 81734506 | 81736552 |
| MER61_3p11.2 | chr3 | 87141103 | 87146130 |
| HERVW_3p11.1 | chr3 | 87940794 | 87946379 |
| HML2_3q12.3 | chr3 | 101691893 | 101701015 |
| MER4_3q13.11 | chr3 | 104346780 | 104352104 |
| ERV316A3_3q13.12c | chr3 | 107248969 | 107249989 |
| HERVH_3q13.2 | chr3 | 112418412 | 112423365 |
| HML2_3q13.2 | chr3 | 113024277 | 113033435 |
| HML2_3q21.2 | chr3 | 125890459 | 125899596 |
| PRIMA41_3q21.3b | chr3 | 128666873 | 128670003 |
| HERVH_3q21.3b | chr3 | 128829426 | 128834942 |
| HERVH_3q21.3c | chr3 | 128836083 | 128842027 |
| HERV3_3q21.3 | chr3 | 128854524 | 128860156 |
| HERVH_3q21.3d | chr3 | 128959818 | 128965400 |
| ERVLB4_3q22.1a | chr3 | 129896346 | 129899192 |
| HML3_3q22.1 | chr3 | 129901262 | 129907337 |
| HERVH_3q22.1b | chr3 | 130137641 | 130140911 |
| HERVE_3q22.1b | chr3 | 130140912 | 130149230 |
| HERVH_3q24d | chr3 | 145551229 | 145556922 |
| HERVL_3q25.1 | chr3 | 151419191 | 151421384 |
| HERVW_3q26.32 | chr3 | 179054591 | 179060155 |
| HML3_3q27.2a | chr3 | 185012756 | 185019807 |
| ERV316A3_3q27.3e | chr3 | 187374111 | 187378966 |
| MER61_3q28 | chr3 | 190549018 | 190554482 |
| MER4_3q29b | chr3 | 195717357 | 195718895 |
| MER61_4p16.3 | chr4 | 94966 | 101337 |
| HERVW_4p16.3 | chr4 | 191763 | 197016 |
| HERVH_4p16.3b | chr4 | 402861 | 409299 |
| HERVL66_4p16.3 | chr4 | 451175 | 455252 |
| HERVH_4p16.1a | chr4 | 8482399 | 8488709 |
| HERVS71_4p16.1a | chr4 | 9068498 | 9075838 |
| HERVH_4p16.1b | chr4 | 9094400 | 9097916 |
| HERVE_4p16.1c | chr4 | 9097917 | 9106252 |
| HML2_4p16.1a | chr4 | 9121786 | 9131367 |
| HERVS71_4p16.1b | chr4 | 9599829 | 9607243 |
| HERVH_4p16.1d | chr4 | 9638973 | 9644753 |
| HML2_4p16.1b | chr4 | 9657956 | 9667550 |
| MER101_4p16.1 | chr4 | 9967357 | 9972151 |
| HERVIP10FH_4p15.32 | chr4 | 15693941 | 15695719 |
| MER41_4p15.2 | chr4 | 22373540 | 22382732 |
| ERVLB4_4p15.2a | chr4 | 26887864 | 26893109 |
| HERVL_4p13a | chr4 | 42526339 | 42531440 |
| MER4_4p12a | chr4 | 48004464 | 48008688 |
| ERVLE_4p12e | chr4 | 48117258 | 48118641 |
| HERV4_4q12 | chr4 | 55938235 | 55948186 |
| ERVLB4_4q12d | chr4 | 56255572 | 56260979 |
| HERVH_4q13.2 | chr4 | 68030946 | 68037573 |
| HERVK11_4q13.2 | chr4 | 69191808 | 69199234 |
| HERV9_4q13.3 | chr4 | 69824563 | 69828660 |
| HERV3_4q13.3b | chr4 | 69828661 | 69833104 |
| ERVLE_4q13.3g | chr4 | 73313544 | 73317374 |
| ERVLE_4q21.1b | chr4 | 76291860 | 76295260 |
| HERVH_4q21.21a | chr4 | 78713842 | 78719721 |
| HUERSP1_4q21.22 | chr4 | 83096768 | 83099235 |
| ERVLE_4q21.23c | chr4 | 84945001 | 84946272 |
| ERVLE_4q21.3 | chr4 | 86916104 | 86921518 |
| HERVH_4q22.1c | chr4 | 88152327 | 88155548 |
| HERV4_4q22.1 | chr4 | 88442739 | 88453401 |
| HERVH_4q22.3a | chr4 | 94293297 | 94298570 |
| MER101_4q23 | chr4 | 98328796 | 98335519 |
| MER4_4q23b | chr4 | 99152149 | 99155660 |
| ERVLE_4q24g | chr4 | 102271200 | 102271959 |
| HERVL_4q24d | chr4 | 102741898 | 102747566 |
| HML5_4q24 | chr4 | 106109837 | 106114668 |
| MER4_4q25a | chr4 | 107883818 | 107884951 |
| MER4_4q25b | chr4 | 107886507 | 107892905 |
| ERVLE_4q25h | chr4 | 112330565 | 112333779 |
| MER61_4q26a | chr4 | 114145863 | 114149406 |
| HERVH_4q31.1b | chr4 | 139157976 | 139160055 |
| ERV316A3_4q31.1b | chr4 | 140289102 | 140293802 |
| MER61_4q31.21a | chr4 | 142656025 | 142661274 |
| HERVP71A_4q31.21 | chr4 | 143177566 | 143180491 |
| MER61_4q31.21b | chr4 | 143426951 | 143432579 |
| HERVH_4q31.21c | chr4 | 145832612 | 145838502 |
| HML3_4q31.3 | chr4 | 153688368 | 153694427 |
| MER34B_4q31.3 | chr4 | 153696581 | 153699612 |
| HARLEQUIN_4q32.1 | chr4 | 158224447 | 158229241 |
| MER41_4q34.1a | chr4 | 171578431 | 171582849 |
| LTR25_4q34.1a | chr4 | 173282514 | 173289284 |
| LTR19_4q35.1 | chr4 | 185324325 | 185330494 |
| HERVH_4q35.2c | chr4 | 189925901 | 189931855 |
| HML3_5p15.33c | chr5 | 1577862 | 1580973 |
| HML3_5p15.33d | chr5 | 1581038 | 1585387 |
| HERVL_5p15.2a | chr5 | 10716384 | 10720094 |
| HERVH_5p14.3a | chr5 | 18668843 | 18674598 |
| HERVW_5p14.3 | chr5 | 18742472 | 18746014 |
| MER61_5p14.1 | chr5 | 27472074 | 27477973 |
| HERVL_5p13.3b | chr5 | 32287158 | 32293609 |
| HERVW_5p13.2a | chr5 | 34062180 | 34063274 |
| MER61_5p13.2 | chr5 | 35717489 | 35723193 |
| HML5_5p13.2 | chr5 | 37086386 | 37094268 |
| HARLEQUIN_5p12a | chr5 | 42813176 | 42819811 |
| MER4_5p12a | chr5 | 42964673 | 42966094 |
| MER4_5p12c | chr5 | 42996067 | 42997591 |
| MER4_5p12d | chr5 | 43020758 | 43026332 |
| MER41_5p12 | chr5 | 44785133 | 44788577 |
| HML3_5p12a | chr5 | 44788578 | 44793529 |
| ERVL_5q11.2 | chr5 | 58980759 | 58983927 |
| ERV316A3_5q12.1b | chr5 | 61172131 | 61174398 |
| HERVL_5q12.3 | chr5 | 65486807 | 65491213 |
| HERVL40_5q12.3 | chr5 | 66091510 | 66092988 |
| ERVLB4_5q13.3b | chr5 | 76627891 | 76628460 |
| HERVH_5q13.3b | chr5 | 76879901 | 76887017 |
| HARLEQUIN_5q14.2 | chr5 | 82267274 | 82273743 |
| MER41_5q15a | chr5 | 94039191 | 94045415 |
| HERVS71_5q15 | chr5 | 95707337 | 95712075 |
| HERVEA_5q22.2 | chr5 | 112523903 | 112532633 |
| HERVH_5q23.2g | chr5 | 126839337 | 126845705 |
| HERV9_5q23.3 | chr5 | 130633383 | 130642483 |
| LTR57_5q31.1 | chr5 | 132281240 | 132287803 |
| ERVLE_5q31.1b | chr5 | 132288665 | 132289427 |
| ERVLE_5q31.1c | chr5 | 132289440 | 132292324 |
| ERV316A3_5q31.3c | chr5 | 142727807 | 142735305 |
| HERVL_5q33.1a | chr5 | 150872210 | 150877598 |
| LTR46_5q33.1 | chr5 | 150986467 | 150993656 |
| HERVIP10FH_5q35.1 | chr5 | 172650610 | 172656348 |
| ERV316A3_5q35.3a | chr5 | 177931460 | 177936750 |
| HARLEQUIN_6p25.2 | chr6 | 2310409 | 2318098 |
| HERVFRD_6p24.2 | chr6 | 11102745 | 11112217 |
| ERVLB4_6p23 | chr6 | 14432680 | 14438711 |
| HERVH_6p22.3a | chr6 | 16259012 | 16264893 |
| MER4_6p22.3d | chr6 | 25128933 | 25136513 |
| HML5_6p22.2 | chr6 | 26257219 | 26260461 |
| HML6_6p22.2 | chr6 | 26288022 | 26296266 |
| HERVL18_6p22.1a | chr6 | 27405363 | 27407723 |
| ERV316A3_6p22.1a | chr6 | 27945679 | 27947350 |
| ERV316A3_6p22.1k | chr6 | 29868623 | 29874102 |
| HML3_6p22.1 | chr6 | 29875650 | 29881829 |
| ERV316A3_6p22.1m | chr6 | 29912749 | 29918216 |
| ERV316A3_6p22.1n | chr6 | 29946920 | 29950412 |
| HERVP71A_6p22.1 | chr6 | 29962489 | 29970366 |
| ERV316A3_6p22.1o | chr6 | 29997951 | 30003286 |
| HERVL_6p21.33b | chr6 | 31429886 | 31436004 |
| ERV316A3_6p21.33c | chr6 | 31463420 | 31469043 |
| HUERSP3_6p21.32 | chr6 | 33117739 | 33125003 |
| HERV9_6p21.2 | chr6 | 38616529 | 38624486 |
| HERVH_6p21.2b | chr6 | 39131513 | 39136520 |
| HML5_6p12.1 | chr6 | 57069873 | 57074762 |
| HERVH_6q12c | chr6 | 63950227 | 63956522 |
| HERVH_6q12f | chr6 | 67880996 | 67889473 |
| HML3_6q13 | chr6 | 70445359 | 70449051 |
| MER61_6q14.1b | chr6 | 77094622 | 77100378 |
| ERV316A3_6q14.1a | chr6 | 77149660 | 77152315 |
| HML1_6q14.1 | chr6 | 78854118 | 78858747 |
| HERVL_6q14.1d | chr6 | 80384055 | 80391236 |
| HERVL_6q14.1f | chr6 | 83010538 | 83017105 |
| MER4_6q14.2 | chr6 | 83969147 | 83975641 |
| HERVW_6q14.3a | chr6 | 84711702 | 84718373 |
| ERVLE_6q21c | chr6 | 109356416 | 109361483 |
| MER41_6q21b | chr6 | 110488048 | 110491199 |
| HML3_6q21a | chr6 | 110499174 | 110506068 |
| HML3_6q21b | chr6 | 110916754 | 110923087 |
| HARLEQUIN_6q21 | chr6 | 111284171 | 111290629 |
| HERVIP10FH_6q22.1 | chr6 | 117615274 | 117622007 |
| HERVH_6q22.1c | chr6 | 117658797 | 117665200 |
| MER41_6q22.31b | chr6 | 118601580 | 118608058 |
| HERV30_6q22.31 | chr6 | 118617102 | 118626802 |
| ERVLE_6q22.31b | chr6 | 118694772 | 118700068 |
| HERVL_6q22.31c | chr6 | 121191685 | 121198343 |
| ERV316A3_6q22.31g | chr6 | 124439889 | 124443608 |
| HERVL18_6q22.31b | chr6 | 125267901 | 125274451 |
| HML1_6q22.32 | chr6 | 125910949 | 125912799 |
| HERVH_6q23.2a | chr6 | 131295357 | 131301196 |
| HERVH_6q23.2b | chr6 | 131323296 | 131329137 |
| HERV9_6q25.1 | chr6 | 150849127 | 150856692 |
| HERVK11_6q25.3 | chr6 | 158296613 | 158303618 |
| LTR19_6q25.3a | chr6 | 158611716 | 158613921 |
| HERVIP10FH_6q25.3 | chr6 | 158729866 | 158733323 |
| MER4_6q26b | chr6 | 161209918 | 161214377 |
| HERVW_6q27b | chr6 | 166891080 | 166898253 |
| MER101_6q27b | chr6 | 166918829 | 166922075 |
| HERVE_7p22.1c | chr7 | 6950409 | 6958745 |
| HERVH_7p22.1c | chr7 | 6958746 | 6962263 |
| HERVH_7p21.1b | chr7 | 20120709 | 20126555 |
| HARLEQUIN_7p15.3 | chr7 | 24789291 | 24795789 |
| HERVL_7p15.2a | chr7 | 26122901 | 26125681 |
| HERVS71_7p14.3 | chr7 | 29636372 | 29646401 |
| MER4_7p14.3a | chr7 | 30567785 | 30571827 |
| MER4_7p14.3b | chr7 | 30579723 | 30584961 |
| HERVH_7p14.3d | chr7 | 32832561 | 32838271 |
| HERVH_7p14.3e | chr7 | 32838742 | 32843539 |
| HERVL18_7p14.2 | chr7 | 36472690 | 36474452 |
| HERV9_7p14.2 | chr7 | 36499269 | 36505045 |
| HERV9_7p14.1 | chr7 | 37202715 | 37205100 |
| HML5_7p14.1 | chr7 | 38159987 | 38164084 |
| HML6_7p13 | chr7 | 43808543 | 43809894 |
| ERV316A3_7p13a | chr7 | 45050816 | 45054421 |
| MER41_7q11.21i | chr7 | 64484420 | 64488772 |
| PRIMA4_7q11.21b | chr7 | 64488773 | 64492642 |
| LTR25_7q11.21 | chr7 | 64508618 | 64516308 |
| HERVH_7q11.21 | chr7 | 64679550 | 64686902 |
| HERVFC1_7q11.21 | chr7 | 64834895 | 64840158 |
| HML3_7q11.21 | chr7 | 64948007 | 64950796 |
| HERV3_7q11.21 | chr7 | 64990324 | 64999936 |
| LTR23_7q11.23a | chr7 | 76623872 | 76624265 |
| HERVH_7q11.23a | chr7 | 77339937 | 77345880 |
| HERV9_7q21.11 | chr7 | 82636877 | 82646011 |
| HERV9_7q21.13 | chr7 | 91447505 | 91456909 |
| HERVW_7q21.2 | chr7 | 92467999 | 92478192 |
| ERV316A3_7q21.3b | chr7 | 94563686 | 94566141 |
| HERVE_7q21.3 | chr7 | 97951319 | 97955393 |
| HARLEQUIN_7q22.1 | chr7 | 100838700 | 100847185 |
| HERVL_7q32.1 | chr7 | 129444201 | 129450234 |
| MER41_7q33 | chr7 | 134086639 | 134091633 |
| HARLEQUIN_7q33a | chr7 | 135171264 | 135175958 |
| HERV3_7q33 | chr7 | 135175955 | 135180551 |
| HERV3_7q34a | chr7 | 138556758 | 138566149 |
| HML2_7q34 | chr7 | 141752118 | 141756138 |
| MER41_7q34b | chr7 | 142703799 | 142708967 |
| LTR25_7q36.1 | chr7 | 149527865 | 149535145 |
| ERV316A3_7q36.1a | chr7 | 150257200 | 150259082 |
| HML3_7q36.1c | chr7 | 152038128 | 152044503 |
| ERVLB4_7q36.3a | chr7 | 156819636 | 156821920 |
| ERVLB4_8p23.1b | chr8 | 7223764 | 7224313 |
| ERVLB4_8p23.1f | chr8 | 7682360 | 7682909 |
| HERVH_8p23.1c | chr8 | 8168723 | 8172260 |
| HERVE_8p23.1h | chr8 | 8172261 | 8180584 |
| HERVI_8p23.1 | chr8 | 8358592 | 8363270 |
| HML2_8p23.1d | chr8 | 12216461 | 12225988 |
| HERVS71_8p23.1b | chr8 | 12519908 | 12527223 |
| HERVK11_8p23.1c | chr8 | 12531005 | 12538042 |
| MER101_8p21.3 | chr8 | 22479930 | 22483803 |
| ERVLB4_8p21.3a | chr8 | 23034222 | 23038768 |
| PABLA_8p21.3a | chr8 | 23049150 | 23056094 |
| ERVLB4_8p21.1b | chr8 | 27662386 | 27664144 |
| ERVLB4_8p12b | chr8 | 30057374 | 30061491 |
| MER41_8p11.22 | chr8 | 39004567 | 39006262 |
| ERVLB4_8p11.22a | chr8 | 39057485 | 39064595 |
| HUERSP3_8q11.1 | chr8 | 47005340 | 47015327 |
| ERV316A3_8q11.21a | chr8 | 49160321 | 49162766 |
| ERVLE_8q11.23b | chr8 | 52650632 | 52654006 |
| HERVL40_8q11.23b | chr8 | 54183988 | 54189099 |
| HERVH48_8q12.3 | chr8 | 63143887 | 63147246 |
| HERVW_8q12.3b | chr8 | 64765352 | 64768361 |
| ERVLE_8q12.3f | chr8 | 64811247 | 64815461 |
| HERVFRD_8q13.2 | chr8 | 67702374 | 67709490 |
| MER4B_8q21.11 | chr8 | 74031305 | 74036087 |
| HERVH_8q22.1a | chr8 | 97175029 | 97180739 |
| HERVH_8q22.1c | chr8 | 97827675 | 97831909 |
| HERVL_8q22.2 | chr8 | 100202983 | 100209403 |
| ERVLE_8q22.3b | chr8 | 103307913 | 103309882 |
| MER61_8q23.1b | chr8 | 109219471 | 109223779 |
| HERVL74_8q24.13 | chr8 | 121510181 | 121514719 |
| HERVH48_8q24.13 | chr8 | 122830053 | 122836867 |
| HERVH_8q24.21a | chr8 | 127431703 | 127437464 |
| ERV316A3_8q24.21c | chr8 | 129265172 | 129266245 |
| HERVH_8q24.22a | chr8 | 131310175 | 131315847 |
| PRIMA41_8q24.3 | chr8 | 143050063 | 143053296 |
| HUERSP3_9p23 | chr9 | 9997607 | 10001203 |
| HERVL_9p21.3a | chr9 | 21846888 | 21852044 |
| HERVFRD_9p21.3b | chr9 | 21921892 | 21931935 |
| MER34B_9p21.3b | chr9 | 21933111 | 21940551 |
| MER41_9p13.3a | chr9 | 33703617 | 33708862 |
| MER4_9p13.3a | chr9 | 35012292 | 35020722 |
| HERVH_9p13.3b | chr9 | 35020808 | 35025134 |
| HERVL_9q21.11a | chr9 | 65245525 | 65249540 |
| ERV316A3_9q21.33 | chr9 | 85630235 | 85631584 |
| HERVL_9q21.33b | chr9 | 85775592 | 85779684 |
| HERVH_9q21.33c | chr9 | 85880935 | 85884510 |
| MER41_9q31.1a | chr9 | 100030133 | 100034561 |
| ERVLE_9q33.2b | chr9 | 121394197 | 121398743 |
| ERVLB4_9q34.11 | chr9 | 129766904 | 129771287 |
| HML3_9q34.13 | chr9 | 131680373 | 131685811 |
| HERVI_9q34.13 | chr9 | 133031580 | 133038234 |
| HERVE_9q34.3 | chr9 | 135437085 | 135444492 |
| HERVL40_9q34.3 | chr9 | 136703426 | 136709202 |
| ERVLE_10p15.1d | chr10 | 5438991 | 5440667 |
| HERVH_10p15.1 | chr10 | 5470768 | 5477012 |
| HML2_10p14 | chr10 | 6824179 | 6833641 |
| HERVE_10p13c | chr10 | 15013301 | 15015356 |
| HERV9_10p13 | chr10 | 17011188 | 17018975 |
| MER4_10p12.33 | chr10 | 17379427 | 17385917 |
| PRIMA4_10p12.1 | chr10 | 26979406 | 26999995 |
| MER61_10q21.3 | chr10 | 65570387 | 65573112 |
| ERV316A3_10q22.1a | chr10 | 69105107 | 69112780 |
| MER4B_10q22.3 | chr10 | 80062283 | 80073050 |
| ERV316A3_10q23.2a | chr10 | 87276938 | 87278337 |
| HERVH_10q23.31b | chr10 | 90081018 | 90086792 |
| MER4_10q23.31b | chr10 | 90087053 | 90092706 |
| HERVH_10q23.31c | chr10 | 90294983 | 90300722 |
| HERVH_10q23.31d | chr10 | 90807289 | 90813258 |
| HUERSP3_10q23.33a | chr10 | 95123975 | 95127447 |
| HUERSP3_10q23.33b | chr10 | 95127496 | 95131937 |
| HUERSP3_10q23.33c | chr10 | 95132811 | 95134563 |
| HERVW_10q24.1 | chr10 | 95717359 | 95724870 |
| HERVH_10q24.31a | chr10 | 100380920 | 100388398 |
| ERVLE_10q25.3a | chr10 | 114802955 | 114807340 |
| MER34B_10q25.3b | chr10 | 116992719 | 116997446 |
| HERVH_10q26.12 | chr10 | 120844932 | 120850561 |
| ERVLB4_10q26.2b | chr10 | 127949338 | 127956187 |
| HERVL_10q26.3b | chr10 | 133495747 | 133502939 |
| HERV3_11p15.5 | chr11 | 322542 | 324821 |
| ERVLB4_11p15.5c | chr11 | 2292785 | 2296646 |
| ERVLB4_11p15.5d | chr11 | 2324940 | 2326799 |
| HERVIP10FH_11p15.5 | chr11 | 2335785 | 2342573 |
| MER4_11p15.4a | chr11 | 3110563 | 3118570 |
| HML2_11p15.4a | chr11 | 3447426 | 3456979 |
| HERVE_11p15.4 | chr11 | 3473529 | 3481711 |
| HERVH_11p15.4b | chr11 | 3481712 | 3485187 |
| HERVS71_11p15.4 | chr11 | 3509348 | 3516789 |
| HERVI_11p15.4 | chr11 | 5808038 | 5809640 |
| HUERSP1_11p15.4a | chr11 | 6039059 | 6042072 |
| HUERSP1_11p15.4b | chr11 | 6686696 | 6694293 |
| HERVH48_11p15.4 | chr11 | 7516499 | 7520474 |
| HERVH_11p15.2 | chr11 | 14524261 | 14529062 |
| ERV316A3_11p15.2a | chr11 | 14622139 | 14625891 |
| ERVLE_11p15.2b | chr11 | 14837735 | 14841560 |
| HUERSP3_11p15.1 | chr11 | 17370374 | 17378687 |
| HERVIP10F_11p14.3 | chr11 | 22992671 | 23000729 |
| ERVL_11p14.3 | chr11 | 23103460 | 23105364 |
| LTR25_11p14.1 | chr11 | 28134547 | 28139969 |
| HUERSP3B_11p12 | chr11 | 36665205 | 36673420 |
| HERVL_11q12.1c | chr11 | 57650629 | 57651896 |
| MER4B_11q12.1 | chr11 | 57719337 | 57731650 |
| HML2_11q12.1 | chr11 | 59000009 | 59005723 |
| HERVL_11q12.1d | chr11 | 59276041 | 59282777 |
| HERVL18_11q12.2 | chr11 | 60167847 | 60169566 |
| HARLEQUIN_11q12.2b | chr11 | 60185446 | 60193188 |
| HERVL_11q12.2 | chr11 | 60216680 | 60223953 |
| HML2_11q12.3b | chr11 | 62375545 | 62383091 |
| ERV316A3_11q13.1a | chr11 | 64449595 | 64450882 |
| ERV316A3_11q13.1b | chr11 | 64451182 | 64458279 |
| ERVLE_11q13.1b | chr11 | 64518333 | 64522556 |
| HERV3_11q13.3 | chr11 | 68867426 | 68874582 |
| HERVS71_11q13.4 | chr11 | 71705986 | 71713426 |
| HERVH_11q13.4a | chr11 | 71737575 | 71741114 |
| HERVE_11q13.4c | chr11 | 71741115 | 71749426 |
| HERVH_11q13.4b | chr11 | 71749427 | 71752695 |
| HERVE_11q13.4d | chr11 | 71886034 | 71890084 |
| HERVE_11q13.4e | chr11 | 71908491 | 71912322 |
| ERVLE_11q13.4g | chr11 | 74912656 | 74918274 |
| MER4_11q14.1 | chr11 | 77576248 | 77585610 |
| ERVLE_11q14.2a | chr11 | 86450540 | 86453375 |
| HERVH_11q21a | chr11 | 94641601 | 94647311 |
| HERV4_11q21 | chr11 | 94650011 | 94657535 |
| MER4_11q21d | chr11 | 94809643 | 94812759 |
| MER4_11q21e | chr11 | 94815022 | 94819826 |
| ERVLE_11q23.3c | chr11 | 118707522 | 118709204 |
| HERVH_11q24.1c | chr11 | 122824428 | 122832822 |
| HERVP71A_12p13.33b | chr12 | 110821 | 112456 |
| HERVP71A_12p13.31a | chr12 | 7588093 | 7593696 |
| HERVP71A_12p13.31b | chr12 | 7600342 | 7606495 |
| HERVL_12p13.31b | chr12 | 7996848 | 8005194 |
| ERVLB4_12p13.31e | chr12 | 8392267 | 8392867 |
| HML5_12p13.31 | chr12 | 8558451 | 8562470 |
| HERVW_12p13.31b | chr12 | 8761414 | 8768468 |
| LTR19_12p13.31 | chr12 | 9139752 | 9144991 |
| MER101_12p13.31b | chr12 | 9657215 | 9660579 |
| HERVEA_12p13.31 | chr12 | 9712931 | 9715636 |
| HERVH_12p13.31d | chr12 | 9833927 | 9836182 |
| ERVLE_12p13.31d | chr12 | 9879671 | 9880497 |
| HERVI_12p13.31 | chr12 | 9902662 | 9911410 |
| HERVH_12p13.31e | chr12 | 9962437 | 9968690 |
| MER4B_12p13.31c | chr12 | 9984964 | 9987795 |
| HERVIP10F_12p13.2 | chr12 | 10369013 | 10372258 |
| MER41_12p13.2 | chr12 | 10653837 | 10661086 |
| ERVLB4_12p13.2b | chr12 | 10960321 | 10961256 |
| HERVL_12p13.2a | chr12 | 11016701 | 11018258 |
| HERVL_12p13.2b | chr12 | 11070735 | 11073773 |
| ERVLE_12p13.2c | chr12 | 11152956 | 11153917 |
| HERVH_12p13.2c | chr12 | 11609767 | 11615294 |
| HERVL_12p13.1c | chr12 | 14482264 | 14494650 |
| ERV316A3_12p12.3c | chr12 | 16443828 | 16447656 |
| MER41_12p12.1b | chr12 | 24856758 | 24862303 |
| HERVL_12p11.23 | chr12 | 26775547 | 26782199 |
| LTR25_12p11.22a | chr12 | 29133833 | 29134615 |
| HERVH_12p11.22a | chr12 | 29134620 | 29140801 |
| LTR25_12p11.22b | chr12 | 29140802 | 29145444 |
| PRIMA4_12p11.21b | chr12 | 31307571 | 31315051 |
| HERVL_12q12b | chr12 | 44042385 | 44047187 |
| MER41_12q13.11 | chr12 | 46253205 | 46258347 |
| HERVL_12q13.11a | chr12 | 46727181 | 46734247 |
| HERVL_12q13.11b | chr12 | 46860882 | 46867418 |
| HERVL_12q13.13 | chr12 | 51796550 | 51803499 |
| HERVH_12q13.2b | chr12 | 54942538 | 54948366 |
| ERV316A3_12q14.1e | chr12 | 62251539 | 62253373 |
| HERVL_12q14.2a | chr12 | 64129134 | 64136221 |
| ERVLE_12q14.3a | chr12 | 66256833 | 66258223 |
| HERVH_12q15b | chr12 | 70085290 | 70090295 |
| MER61_12q15a | chr12 | 70467962 | 70472019 |
| MER4B_12q21.2 | chr12 | 75363706 | 75367277 |
| HERVL_12q21.2a | chr12 | 75371001 | 75377839 |
| HERVH_12q21.31 | chr12 | 80783123 | 80788892 |
| HML3_12q22 | chr12 | 94355584 | 94359308 |
| HERVH_12q22 | chr12 | 95748862 | 95754890 |
| HML5_12q23.1 | chr12 | 100159365 | 100165461 |
| HML6_12q24.12a | chr12 | 111816175 | 111824274 |
| ERV316A3_12q24.13 | chr12 | 112974379 | 112975753 |
| HUERSP1_12q24.31b | chr12 | 122709248 | 122711007 |
| HML2_12q24.33 | chr12 | 133090536 | 133096478 |
| ERVLE_13q12.11 | chr13 | 21311505 | 21314276 |
| ERVLB4_13q12.12a | chr13 | 22927361 | 22931755 |
| ERVLE_13q12.13b | chr13 | 27199770 | 27203628 |
| PRIMA41_13q13.1 | chr13 | 31715164 | 31717353 |
| HERVH_13q13.3a | chr13 | 36316325 | 36322191 |
| ERV316A3_13q13.3c | chr13 | 37142248 | 37147742 |
| HERVH_13q13.3b | chr13 | 37969007 | 37974554 |
| HERVE_13q14.13b | chr13 | 45382637 | 45383907 |
| ERVLE_13q14.13 | chr13 | 45384069 | 45386407 |
| HERVL40_13q32.1 | chr13 | 96019147 | 96021285 |
| HERV4_13q32.3 | chr13 | 99486866 | 99497078 |
| HML5_13q33.1 | chr13 | 102667824 | 102672563 |
| ERV316A3_13q33.3b | chr13 | 109465321 | 109466932 |
| HERV9_13q34 | chr13 | 111189214 | 111199112 |
| HERVL18_14q11.2 | chr14 | 20600302 | 20607318 |
| HERVIP10F_14q11.2c | chr14 | 20710435 | 20719019 |
| HERVH_14q11.2b | chr14 | 20814518 | 20816730 |
| HERVIP10FH_14q11.2 | chr14 | 20822717 | 20828859 |
| HERVW_14q11.2 | chr14 | 22236551 | 22244186 |
| ERVLE_14q11.2d | chr14 | 22369972 | 22372430 |
| HML2_14q11.2 | chr14 | 24009696 | 24015776 |
| ERVLE_14q12b | chr14 | 24501756 | 24503008 |
| ERVLE_14q22.1 | chr14 | 50539759 | 50544229 |
| LTR57_14q22.1 | chr14 | 50544972 | 50550643 |
| HERVH_14q22.1a | chr14 | 50893162 | 50896882 |
| PRIMA4_14q22.1 | chr14 | 52280360 | 52286612 |
| HERVH_14q22.1b | chr14 | 53128745 | 53135552 |
| MER4_14q22.3a | chr14 | 56431040 | 56437062 |
| HERVIP10FH_14q23.1 | chr14 | 61114842 | 61121155 |
| HML6_14q24.2 | chr14 | 69811463 | 69816023 |
| HERVH_14q24.2a | chr14 | 70293487 | 70301929 |
| MER4_14q24.2c | chr14 | 70311202 | 70314961 |
| HERVH48_14q24.2 | chr14 | 70344652 | 70350485 |
| HERVH_14q24.2b | chr14 | 71124207 | 71130006 |
| ERV316A3_14q24.2f | chr14 | 71132407 | 71138320 |
| HERVL_14q24.2c | chr14 | 71459383 | 71466120 |
| HML5_14q31.1 | chr14 | 80464388 | 80473042 |
| ERVLB4_14q31.3b | chr14 | 86017276 | 86020045 |
| ERVLB4_14q32.11b | chr14 | 91016539 | 91019592 |
| HERVH_14q32.2c | chr14 | 98985205 | 98992939 |
| MER4_14q32.2 | chr14 | 100000066 | 100007471 |
| HERVFH21_14q32.31a | chr14 | 102233835 | 102239533 |
| HERVH_14q32.31b | chr14 | 102239957 | 102245769 |
| HERVH_14q32.31c | chr14 | 102410504 | 102411706 |
| MER41_14q32.32 | chr14 | 102856187 | 102861776 |
| MER4_14q32.33a | chr14 | 105031317 | 105039940 |
| HERVS71_14q32.33 | chr14 | 106196991 | 106207472 |
| HML5_14q32.33b | chr14 | 106744465 | 106749566 |
| HERV3_14q32.33 | chr14 | 106836061 | 106845981 |
| MER4B_14q32.33d | chr14 | 106852618 | 106853459 |
| ERVLB4_15q13.2c | chr15 | 30867400 | 30872949 |
| HERVIP10F_15q14 | chr15 | 34154726 | 34161977 |
| LTR25_15q21.1a | chr15 | 45049247 | 45052678 |
| HML5_15q21.2 | chr15 | 49839371 | 49844779 |
| MER4_15q21.2a | chr15 | 51416001 | 51417137 |
| ERVLE_15q21.2 | chr15 | 52113318 | 52114980 |
| HERVW_15q21.3 | chr15 | 55304882 | 55312376 |
| MER41_15q22.31b | chr15 | 66464103 | 66466989 |
| HERV4_15q26.1 | chr15 | 89462545 | 89469801 |
| ERVLE_15q26.3b | chr15 | 99383589 | 99384407 |
| HERVH_15q26.3a | chr15 | 100440257 | 100446201 |
| HERVH_15q26.3b | chr15 | 101072487 | 101078293 |
| ERVLB4_16p13.3a | chr16 | 1935021 | 1937225 |
| HML3_16p13.3 | chr16 | 3076839 | 3081509 |
| HML3_16p13.13 | chr16 | 10417952 | 10424337 |
| HERVI_16p13.13 | chr16 | 10446457 | 10447680 |
| MER101_16p12.2a | chr16 | 21427157 | 21428254 |
| ERVL_16p12.2 | chr16 | 23992260 | 23998394 |
| HML5_16p11.2 | chr16 | 31763078 | 31767906 |
| HERVL_16q24.1 | chr16 | 84855240 | 84861709 |
| HML3_17p13.1 | chr17 | 6875146 | 6881530 |
| HERV4_17p13.1 | chr17 | 6936343 | 6940666 |
| HERVE_17q11.2 | chr17 | 28230519 | 28239339 |
| HERVL_17q12a | chr17 | 35453456 | 35456843 |
| HERVL74_17q12 | chr17 | 35464720 | 35470123 |
| HARLEQUIN_17q12 | chr17 | 35500743 | 35508365 |
| HERVL_17q12b | chr17 | 36111494 | 36116344 |
| MER4_17q21.2d | chr17 | 41109018 | 41116054 |
| HARLEQUIN_17q21.31 | chr17 | 43160246 | 43167698 |
| ERVLB4_17q21.32 | chr17 | 49202488 | 49204391 |
| ERV316A3_17q21.33a | chr17 | 50317558 | 50319950 |
| ERV316A3_17q21.33c | chr17 | 50324431 | 50326228 |
| HERVH_17q25.1a | chr17 | 74503255 | 74506420 |
| ERVLB4_17q25.1 | chr17 | 74540590 | 74541302 |
| HERVH_17q25.3 | chr17 | 80399794 | 80408186 |
| HARLEQUIN_17q25.3b | chr17 | 80408187 | 80413516 |
| HERVFRD_18p11.23 | chr18 | 8177580 | 8180999 |
| HARLEQUIN_18p11.22a | chr18 | 8913647 | 8921365 |
| HARLEQUIN_18p11.22b | chr18 | 10599605 | 10604201 |
| HERVL_18p11.21a | chr18 | 11127309 | 11133941 |
| ERV316A3_18q11.2c | chr18 | 24356917 | 24358507 |
| HERV9_18q11.2 | chr18 | 26231675 | 26238550 |
| ERVLE_18q11.2c | chr18 | 26369428 | 26373198 |
| MER61_18q12.3a | chr18 | 39795158 | 39800435 |
| ERV316A3_18q21.1b | chr18 | 46275058 | 46278556 |
| MER61_18q21.2b | chr18 | 54382339 | 54387923 |
| PRIMA4_18q23b | chr18 | 80023832 | 80033162 |
| PABLA_18q23 | chr18 | 80060215 | 80064591 |
| HERV3_19p13.3 | chr19 | 566440 | 568668 |
| HERVS71_19p13.3 | chr19 | 6656135 | 6661723 |
| HERVH_19p13.2a | chr19 | 9364148 | 9366007 |
| HERVIP10F_19p13.2b | chr19 | 9718757 | 9722354 |
| HERVEA_19p13.2 | chr19 | 9725353 | 9730899 |
| HERVL74_19p13.2b | chr19 | 9737195 | 9740103 |
| HERVH_19p13.2d | chr19 | 9741826 | 9751972 |
| HML6_19p13.2d | chr19 | 11853282 | 11860984 |
| HML3_19p13.2d | chr19 | 12098326 | 12102949 |
| HML3_19p13.2e | chr19 | 12103011 | 12104292 |
| LTR19_19p13.2b | chr19 | 12224330 | 12228688 |
| HML1_19p13.2 | chr19 | 12450589 | 12454543 |
| HERVH_19p13.12 | chr19 | 15828738 | 15834725 |
| MER34B_19p13.11 | chr19 | 18180092 | 18184013 |
| HERVEA_19p13.11 | chr19 | 19895560 | 19897814 |
| HERVIP10F_19p13.11 | chr19 | 19897815 | 19900640 |
| HERVFH21_19p12 | chr19 | 19943851 | 19951066 |
| HERVL66_19p12a | chr19 | 19959694 | 19964935 |
| HERVH_19p12a | chr19 | 20087687 | 20096781 |
| HERVW_19p12a | chr19 | 20099536 | 20101469 |
| HERVIP10FH_19p12c | chr19 | 20857639 | 20862513 |
| HARLEQUIN_19p12b | chr19 | 21002382 | 21009375 |
| HML3_19p12b | chr19 | 21412023 | 21417699 |
| HML3_19p12c | chr19 | 21417759 | 21419018 |
| HERV3_19p12b | chr19 | 21430674 | 21441176 |
| MER4_19p12a | chr19 | 21454300 | 21456510 |
| HML5_19p12a | chr19 | 21621638 | 21627353 |
| HERVW_19p12b | chr19 | 21629385 | 21634782 |
| HERVS71_19p12b | chr19 | 21688200 | 21697956 |
| MER61_19p12c | chr19 | 23103226 | 23105106 |
| HERVH48_19p12b | chr19 | 23344732 | 23350997 |
| HERV4_19p12 | chr19 | 23666052 | 23671676 |
| HERV3_19p12e | chr19 | 23817565 | 23824289 |
| HML6_19p12e | chr19 | 23864829 | 23871228 |
| HML1_19p12c | chr19 | 23950435 | 23956522 |
| PRIMA41_19p12d | chr19 | 24079035 | 24082362 |
| HUERSP3_19q11 | chr19 | 27876948 | 27885917 |
| HERVE_19q12 | chr19 | 28606436 | 28615273 |
| LTR25_19q13.11 | chr19 | 34621011 | 34625016 |
| HERVH_19q13.12a | chr19 | 36155475 | 36161023 |
| HERVH_19q13.12b | chr19 | 36259508 | 36265220 |
| HERVS71_19q13.12a | chr19 | 36324873 | 36331043 |
| ERVLB4_19q13.12 | chr19 | 36669468 | 36673749 |
| HERVS71_19q13.12b | chr19 | 37554746 | 37558628 |
| HERVH_19q13.2a | chr19 | 38750366 | 38755295 |
| MER101_19q13.2c | chr19 | 40110255 | 40116410 |
| HUERSP1_19q13.2a | chr19 | 41532323 | 41532993 |
| HUERSP2_19q13.2 | chr19 | 41536367 | 41540526 |
| PABLA_19q13.2 | chr19 | 41551919 | 41554973 |
| ERVLE_19q13.2b | chr19 | 42029596 | 42031584 |
| MER41_19q13.2a | chr19 | 42487086 | 42492400 |
| HERVH_19q13.31 | chr19 | 43323511 | 43329384 |
| ERVLE_19q13.31a | chr19 | 43364281 | 43366105 |
| ERVLE_19q13.31c | chr19 | 44181562 | 44184726 |
| ERVLE_19q13.31d | chr19 | 44195798 | 44196936 |
| HML3_19q13.31 | chr19 | 44631504 | 44634709 |
| HERVH_19q13.32e | chr19 | 47392829 | 47398756 |
| HML6_19q13.41f | chr19 | 51804696 | 51811939 |
| HERV4_19q13.41 | chr19 | 52059698 | 52062168 |
| HML6_19q13.41c | chr19 | 52410183 | 52414733 |
| HERVKC4_19q13.41 | chr19 | 52460867 | 52466497 |
| HERVIP10F_19q13.41a | chr19 | 52649405 | 52651539 |
| HML6_19q13.41e | chr19 | 52984535 | 52989576 |
| ERVLB4_19q13.42a | chr19 | 54568262 | 54571051 |
| HUERSP3_19q13.42 | chr19 | 54672643 | 54677704 |
| HERVH_19q13.42b | chr19 | 54894297 | 54900597 |
| HML6_19q13.43a | chr19 | 57512616 | 57518488 |
| LTR46_19q13.43 | chr19 | 57651316 | 57656645 |
| HERV4_19q13.43a | chr19 | 58248684 | 58250591 |
| HERV4_19q13.43b | chr19 | 58251074 | 58253959 |
| HERVH_20p13c | chr20 | 1727239 | 1733570 |
| ERVLB4_20p12.3c | chr20 | 8836313 | 8839500 |
| HERVH_20p12.1c | chr20 | 13357880 | 13362689 |
| HERVE_20p11.21b | chr20 | 24927945 | 24933027 |
| HML2_20q11.22 | chr20 | 34127754 | 34136578 |
| HERVH_20q13.12a | chr20 | 44670229 | 44676774 |
| HERVEA_21q21.1 | chr21 | 19995182 | 20000184 |
| HERVL_21q21.1e | chr21 | 20102058 | 20104865 |
| ERV316A3_21q21.2g | chr21 | 24438073 | 24441244 |
| HERVEA_21q22.11 | chr21 | 32659369 | 32664759 |
| HERVL_21q22.2 | chr21 | 41189736 | 41194314 |
| HERVH_21q22.3 | chr21 | 42800847 | 42806578 |
| MER4_21q22.3a | chr21 | 42812949 | 42815759 |
| HERVH48_21q22.3 | chr21 | 42916742 | 42925876 |
| HUERSP2_21q22.3 | chr21 | 44485340 | 44491111 |
| ERV316A3_21q22.3e | chr21 | 46451755 | 46458103 |
| HERVH_22q11.1 | chr22 | 16611308 | 16615149 |
| ERVLB4_22q11.21a | chr22 | 19787055 | 19787825 |
| HML2_22q11.23 | chr22 | 23536062 | 23546900 |
| ERVL_22q12.3b | chr22 | 36135027 | 36136798 |
| MER41_22q12.3e | chr22 | 37188838 | 37196108 |
| ERV316A3_22q13.1 | chr22 | 37287909 | 37290999 |
| ERV316A3_22q13.33a | chr22 | 49930842 | 49933439 |
| HERVH_Xp22.33 | chrX | 3553142 | 3560161 |
| HUERSP1_Xp22.2 | chrX | 9665455 | 9670571 |
| MER4_Xp22.2d | chrX | 16577951 | 16583715 |
| HERVIP10FH_Xp21.1 | chrX | 37480049 | 37484837 |
| ERVLB4_Xp11.23b | chrX | 48756178 | 48759374 |
| HERVE_Xp11.23 | chrX | 49139563 | 49149008 |
| HML6_Xp11.22 | chrX | 53159114 | 53163826 |
| ERVLB4_Xp11.21a | chrX | 55741699 | 55747805 |
| HML6_Xp11.21 | chrX | 57102981 | 57109396 |
| HML3_Xp11.21 | chrX | 57203018 | 57209051 |
| HERVW_Xp11.21 | chrX | 57398559 | 57405610 |
| MER41_Xq11.1c | chrX | 63237441 | 63244731 |
| HERVL_Xq11.1a | chrX | 63309763 | 63317045 |
| ERVLB4_Xq11.1b | chrX | 63322035 | 63327271 |
| HML5_Xq11.1 | chrX | 63358013 | 63364629 |
| HERVH_Xq13.1a | chrX | 71264380 | 71269610 |
| HERVH_Xq13.1b | chrX | 71270272 | 71272831 |
| HUERSP3B_Xq13.2b | chrX | 74253889 | 74260438 |
| MER4B_Xq13.3 | chrX | 76146255 | 76148830 |
| HML3_Xq21.1a | chrX | 77077638 | 77081927 |
| ERVL_Xq21.1a | chrX | 79351332 | 79353473 |
| MER4_Xq21.1a | chrX | 79455176 | 79462438 |
| HERVFH21_Xq21.1 | chrX | 84738500 | 84745112 |
| HERVH_Xq22.2b | chrX | 103761159 | 103762899 |
| HERVH_Xq22.2c | chrX | 103763061 | 103767306 |
| HERVL40_Xq22.2 | chrX | 103857699 | 103863417 |
| LTR25_Xq22.2b | chrX | 103873422 | 103879991 |
| HERVL_Xq23b | chrX | 113760400 | 113765460 |
| MER4B_Xq23a | chrX | 114052360 | 114059021 |
| HARLEQUIN_Xq23b | chrX | 115916433 | 115924222 |
| MER101_Xq23b | chrX | 115936603 | 115939995 |
| HERVIP10FH_Xq24 | chrX | 120143043 | 120145407 |
| MER4_Xq24d | chrX | 120227768 | 120235797 |
| MER4B_Xq26.2a | chrX | 131689719 | 131692783 |
| MER4B_Xq26.2b | chrX | 131692832 | 131696311 |
| HERVH_Xq26.2 | chrX | 131712025 | 131717764 |
| HML1_Xq26.3 | chrX | 135745315 | 135747484 |
| ERVLE_Xq26.3h | chrX | 135747490 | 135749792 |
| ERV316A3_Xq28i | chrX | 153034407 | 153039482 |
| MER4B_Yq11.221a | chrY | 12668114 | 12677969 |
| MER41_Yq11.221a | chrY | 12693892 | 12697964 |
| HUERSP2_Yq11.221 | chrY | 12922349 | 12926718 |
| HML3_Yq11.221b | chrY | 13252527 | 13259365 |
| HERVW_Yq11.222b | chrY | 19079938 | 19087497 |
| LTR25_Yq11.223d | chrY | 20398315 | 20413068 |
| HUERSP2_Yq11.223 | chrY | 20447642 | 20451745 |
| HUERSP3_Yq11.223a | chrY | 20465957 | 20469876 |
| LTR25_Yq11.223g | chrY | 21138826 | 21141259 |
| LTR19_Yq11.223a | chrY | 21141258 | 21146830 |
| LTR25_Yq11.223h | chrY | 21146832 | 21149903 |
| HERV3_Yq11.223b | chrY | 21240702 | 21249208 |
| HERVL66_17_GL000205v2_random(a) | chr17_GL000205v2_random | 40392 | 43993 |

Table 2 summarizes the genomic coordinates (first nucleotide and last nucleotide) of each of the 703 HERV sequences in the GRCH38 version of the human genome.

For example, concerning the herv_id “ERV316A3_1q25.2b”, the “1” value corresponds to chromosome 1 of the human genome, the letter “q” corresponds to the long arm of the corresponding chromosome (alternatively the letter (p) corresponds to the short arm of the corresponding chromosome) and “25.2b” corresponds to the locus of the gene of the corresponding chromosome.

### REFERENCES

1. Consortium IHGS. Initial sequencing and analysis of the human genome. Nature. 15 févr 2001; 409 (6822): 35057062.
2. Johnson W E. Origins and evolutionary consequences of ancient endogenous retroviruses. Nat Rev Microbiol. June 2019; 17 (6): 355-70.
3. Vargiu L, et al. Classification and characterization of human endogenous retroviruses; mosaic forms are common. Retrovirology. 22 janv 2016; 13 (1): 7.
4. Kassiotis G, Stoye J P. Immune responses to endogenous retroelements: taking the bad with the good. Nat Rev Immunol. avr 2016; 16 (4): 207-19.
5. Alcazer V et al. Human Endogenous Retroviruses (HERVs): Shaping the Innate Immune Response in Cancers. Cancers (Basel). 6 mars 2020; 12 (3).
6. Larouche J-D et al. Widespread and tissue-specific expression of endogenous retroelements in human somatic tissues. Genome Med. déc 2020; 12 (1): 1-16.
7. Burns K H. Transposable elements in cancer. Nat Rev Cancer. juill 2017; 17 (7): 415-24.
8. Attermann A S et al. Human endogenous retroviruses and their implication for immunotherapeutics of cancer. Ann Oncol. 01 2018; 29 (11): 2183-91.
9. De Kouchkovsky I, Abdul-Hay M. ‘Acute myeloid leukemia: a comprehensive review and 2016 update’. Blood Cancer Journal. juill 2016; 6 (7): e441-e441.
10. Herold T et al. A 29-gene and cytogenetic score for the prediction of resistance to induction treatment in acute myeloid leukemia. Haematologica. 2018; 103 (3): 456-65.
11. Alexandrov L B et al. Signatures of mutational processes in human cancer. Nature. 22 août 2013; 500 (7463): 415-21.
12. Smith C C et al. Alternative tumour-specific antigens. Nat Rev Cancer. août 2019; 19 (8): 465-78.
13. Brodsky I et al. Expression of HERV-K proviruses in human leukocytes. Blood. 1 mai 1993; 81 (9): 2369-74.
14. Depil S et al. Expression of a human endogenous retrovirus, HERV-K, in the blood cells of leukemia patients. Leukemia. févr 2002; 16 (2): 254-9.
15. Tobiasson M et al. Comprehensive mapping of the effects of azacitidine on DNA methylation, repressive/permissive histone marks and gene expression in primary cells from patients with MDS and MDS-related disease. Oncotarget. 25 avr 2017; 8 (17): 28812-25.
16. Kazachenka A et al. Epigenetic therapy of myelodysplastic syndromes connects to cellular differentiation independently of endogenous retroelement derepression. Genome Medicine. 23 déc 2019; 11 (1): 86.
17. Deniz Ö et al. Endogenous retroviruses are a source of enhancers with oncogenic potential in acute myeloid leukaemia. Nat Commun. 14 2020; 11 (1): 3506.
18. Bendall M L et al. Telescope: Characterization of the retrotranscriptome by accurate estimation of transposable element expression. PLOS Comput Biol. 2019; 15 (9): e1006453.
19. Corces, M. R. et al. (2016). Lineage-specific and single-cell chromatin accessibility charts human hematopoiesis and leukemia evolution. Nat. Genet. 48, 1193-1203.
20. Sondka Z. et al. (2018). The COSMIC Cancer Gene Census: describing genetic dysfunction across all human cancers. Nat. Rev. Cancer 18, 696-705.
21. Tyner J. W. et al. (2018). Functional genomic landscape of acute myeloid leukaemia. Nature 562, 526.
22. Langmead, B., and Salzberg, S. L. (2012). Fast gapped-read alignment with Bowtie 2. Nat. Methods 9, 357-359.
23. Li, H., Handsaker et al., and 1000 Genome Project Data Processing Subgroup (2009). The Sequence Alignment/Map format and SAMtools. Bioinformatics 25, 2078-2079.
24. Anders, S., Pyl, P. T., and Huber, W. (2015). HTSeq—a Python framework to work with high-throughput sequencing data. Bioinformatics 31, 166-169.
25. Love, M. I., Huber, W., and Anders, S. (2014). Moderated estimation of fold change and dispersion for RNA-seq data with DESeq2. Genome Biol. 15, 550.
26. Liberzon A. et al. (2015). The Molecular Signatures Database Hallmark Gene Set Collection. Cell Syst. 1, 417-425.
27. Loeffler-Wirth H et al. (2019). A modular transcriptome map of mature B cell lymphomas. Genome Med. 11, 27.
28. Hubert, M. et al. (2020). IFN-III is selectively produced by cDC1 and predicts good clinical outcome in breast cancer. Sci. Immunol. 5.
29. Thorsson et al. (2018). The Immune Landscape of Cancer. Immunity 48, 812-830.e14.
30. Zhu, A. et al. (2019). Heavy-tailed prior distributions for sequence count data: removing the noise and preserving large differences. Bioinformatics 35, 2084-2092.
31. The Cancer Genome Atlas Research Network (2013). Genomic and Epigenomic Landscapes of Adult De Novo Acute Myeloid Leukemia. N. Engl. J. Med. 368, 2059-2074.
32. H. Kantarjian et al., Blood Cancer Journal 11, 41 (2021).

1-8. (canceled)

9. A method for attributing an AML patient to an AML subtype among 9 AML subtypes characterized by their specific HERVs listed in Table 1 with the indication of their herv_id, the method comprising providing relationship between said 9 AML sub-types and HERVs characterized by their specific herv_id and their relationship with one of these AML subtypes, as set forth in Table 1, determining from a patient cell sample HERVs expression profile, determining which of the 9 AML subtypes is the most represented based on HERV expression in said cell sample, and attributing to the patient the most represented AML subtype among the 9 AML subtypes.

10. The method according to claim 9, comprising
a) determining from a patient's sample the expression value of the 703 HERVs listed in Table 1, or of a sub-part comprising the HERVs with a coefficient >1.2 and those with a coefficient <0.8, as indicated in Table 1,
b) multiplying each HERV expression value by the coefficient attributed to the corresponding HERV in Table 1,
c) for each of the 9 AML subtypes, calculate their score as the mean of each HERV expression specific to the subtype, and
d) attributing to the patient the AML subtype with the highest score among the 9 AML subtypes.

11. The method according to claim 10, wherein step a) comprises performing RNA-Seq, in a sample of a patient, method in which RNA from the sample is fragmented and the fragments are reverse transcribed into cDNA fragments, or the RNA is reverse transcribed to cDNA and then fragmented to get cDNA fragments.

12. The method according to claim 11, wherein step a) comprises performing generation sequencing (NGS) in a sample of a patient.

13. The method according to claim 11, wherein the sample of the patient is a bulk bone marrow sample.

14. The method according to claim 11, wherein in step a) cDNA fragments are sequenced and aligned back to a pre-sequenced reference human genome or human genome reference, using a sequence aligner, these alignments are tested for overlap with said HERVs' sequences, and the number of overlap reads mapped to a gene is registered for each HERVs' sequence giving its expression value.

15. The method according to claim 9, further attributing a prognosis to the patient relative to AML, say, if the patient is attributed AML subtype 1 or 9, prognosis is good, if he is attributed AML subtype 2 or 7, prognosis is medium good, and if he is attributed AML subtype 8, 4, 3, 6 or 5, prognosis is bad.

16. The method according to claim 15, further comprising the recommendation of treating said patient with a cancer therapy against AML, preferably an aggressive therapy, preferably intensified chemotherapy or an alternative therapy through enrollment into a clinical trial for a novel therapy, when the patient is attributed AML subtype 2 or 7, or AML subtype 8, 4, 3, 6 or 5.

17. The method according to claim 15, further comprising the recommendation of treating said patient with an aggressive therapy, preferably intensified chemotherapy or an alternative therapy through enrollment into a clinical trial for a novel therapy, if the patient is attributed AML subtype 8, 4, 3, 6 or 5, or with a less aggressive therapy, preferably standard chemotherapy, if the patient is attributed AML subtype 9, 1, 2 or 7.

18. A method of treating AML in a patient, comprising the steps of:
1) attributing said patient to an AML subtype according to the method according to claim 1, and
2) treating said patient with a cancer therapy depending on the AML subtype.

19. The method according to claim 18, wherein the patient is treated with a cancer therapy when the patient is attributed AML subtype 2, 3, 4, 5, 6, 7 or 8.

20. The method according to claim 19, wherein the cancer therapy is an aggressive therapy.

21. The method according to claim 20, wherein the aggressive therapy is an intensified chemotherapy or an alternative therapy through enrollment into a clinical trial for a novel therapy.

22. The method according to claim 18, wherein the patient is treated with an aggressive therapy if the patient is attributed AML subtype 8, 4, 3, 6 or 5, or a less aggressive therapy if the patient is attributed AML subtype 9, 1, 2 or 7.

23. The method according to claim 22, wherein the aggressive therapy is an intensified chemotherapy or an alternative therapy through enrollment into a clinical trial for a novel therapy.

24. The method according to claim 22, wherein the less aggressive therapy is a standard chemotherapy.

25. The method according to claim 18, wherein the therapy is selected from cytarabine, fludarabine, idarubicin, avapritinib, dasatinib, mitoxantrone, clofarabine, cladribine, azacitidine, daunorubicin, etoposide, midostaurin, sorafenib, gilteritinib, decitabine, lomustine, quizartinib, crenolanib, enasidenib, ivosidenib, venetoclax, glasdegib, antibodies such as gemtuzumab, magrolimab, and combinations thereof.

26. A method of treating AML in a patient, comprising the steps of:
1) attributing said patient to an AML subtype according to the method according to claim 1, and
2) attributing a prognosis to the patient relative to AML, say, if the patient is attributed AML subtype 1 or 9, prognosis is good, if he is attributed AML subtype 2 or 7, prognosis is medium good, and if he is attributed AML subtype 8, 4, 3, 6 or 5, prognosis is bad, and
3) treating said patient with a cancer therapy when the subject had been previously identified as being in a medium good or bad prognosis.

27. The method according to claim 26, wherein the cancer therapy is an aggressive therapy.

28. The method according to claim 27, wherein the aggressive therapy is an intensified chemotherapy or an alternative therapy through enrollment into a clinical trial for a novel therapy.
